(19) United States
(12) Patent Application Publication
(10) Pub. No.: US 2026/0345802 A1
(43) Pub. Date: Nov. 5, 2026

(54) ANTIBODY-DRUG CONJUGATES AND LINKERS AND METHODS OF USE THEREOF

(71) Applicant: Ohio State Innovation Foundation, Columbus, OH (US)

(72) Inventors: Xiaoguang LIU, Columbus, OH (US); Lufang Zhou, Columbus, OH (US)

(73) Assignee: Ohio State Innovation Foundation, Columbus, OH (US)

(21) Appl. No.: 19/447,560

(22) Filed: Jan. 13, 2026

Related U.S. Application Data
(63) Continuation of application No. PCT/US2024/037838, filed on Jul. 12, 2024.
(60) Provisional application No. 63/513,635, filed on Jul. 14, 2023.

(51) Int. Cl. A61K 47/68; C07K 16/28

(52) U.S. Cl. CPC A61K 47/68031; A61K 47/6849; A61K 47/6889; C07K 16/2827

(57) ABSTRACT

The present disclosure provides for an antibody-drug conjugate comprising a monoclonal antibody (mAb), a first payload conjugated to the mAb via a first bivalent linker, and a second payload conjugated to the mAb via a second bivalent linker; wherein the first linker, second linker, or any combination thereof, comprise a phosphine-azide linker, a N-succinimidyl S-acetylthioacetate sulfo-succinimidyl-4-(N-maleimidomethyl)cyclohexane-1-carboxylate (SATA sulfo-SMCC) linker, a sulfo-succinimidyl-4-(N-maleimidomethyl)cyclohexane-1-carboxylate (sulfo-SMCC) linker, or a dibromomaleimide (DBM) linker. Further provided herein are compounds, compositions, and methods relating thereto.

### CROSS-REFERENCE TO RELATED APPLICATIONS

This application claims the benefit of priority to, and the benefit of, U.S. Provisional Application No. 63/513,635 filed on Jul. 14, 2023, the disclosure of which is hereby expressly incorporated by reference herein in its entirety.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Jul. 10, 2024, is entitled “103361-478WO1_ST26.xml”, and is 77,922 bytes in size.

### BACKGROUND

Traditional antibody-drug conjugates only carry a single payload, thereby prohibiting targeting different types of cells to improve subject treatment outcomes. For example, issues can present with circulation stability, side effects, and anti-cancer efficacy, especially for highly aggressive and heterogenous cancers. The use of only a single payload eliminates any ability to optimize or adjust the conjugate for maximizing anti-cancer efficacy. Further, there is no way of increasing tumoral immunity in addition to the cancer killing effect.

The compositions and methods disclosed herein address these and other needs.

### SUMMARY

In accordance with the purposes of the disclosed materials and methods, as embodied and broadly described herein, the disclosed subject matter, in one aspect, relates to bioconjugation and linkers relating thereto.

Thus, in one example, an antibody-drug conjugate is provided, comprising a monoclonal antibody (mAb), a first payload conjugated to the mAb via a first bivalent linker, and a second payload conjugated to the mAb via a second bivalent linker; wherein the first linker, second linker, or any combination thereof, comprise a phosphine-azide linker, a N-succinimidyl S-acetylthioacetate sulfo-succinimidyl-4-(N-maleimidomethyl)cyclohexane-1-carboxylate (SATA sulfo-SMCC) linker, a sulfo-succinimidyl-4-(N-maleimidomethyl)cyclohexane-1-carboxylate (sulfo-SMCC) linker, or a dibromomaleimide (DBM) linker.

In a further example, method of treating a disease in a subject in need thereof is provided comprising administering to the subject a therapeutically effective amount of the antibody-drug conjugate disclosed herein.

A method of upregulating tumoral immunity of a subject in need thereof is further provided herein comprising administering to the subject a therapeutically effective amount of the antibody-drug conjugate disclosed herein.

A method of reducing growth of a tumor in a subject in need thereof is also provided herein comprising administering to the subject a therapeutically effective amount of the antibody-drug conjugate disclosed herein.

A method of inhibiting a protein in a cell in need thereof is further provided comprising administering to the cell a therapeutically effective amount of the antibody-drug conjugate disclosed herein, wherein the antibody in the antibody-drug conjugate inhibits the protein.

Also provided herein is an antibody-drug conjugate comprising a monoclonal antibody (mAb) and a payload conjugated to the mAb via a bivalent linker, wherein the bivalent linker comprises a phosphine-azide linker or a N-succinimidyl S-acetylthioacetate sulfo-succinimidyl-4-(N-maleimidomethyl)cyclohexane-1-carboxylate (sulfo-SMCC) (SATA sulfo-SMCC) linker.

Further provided herein is a compound according to Formula I:

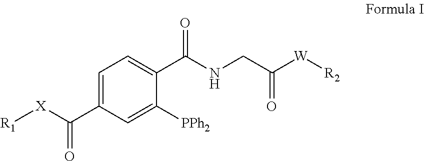

wherein R₁ and R₂ are selected in combination such that one of R₁ and R₂ comprises an antibody and the other of R₁ and R₂ comprises a drug; X is selected from —O—, —S—, and —NH-amine, amide, ester, or any combination thereof; and W is selected from —O—, —S—, and —NH—.

Also provided herein is a method of covalently linking a first payload to a second payload, the method comprising: reacting the first payload with a compound according to Formula III:

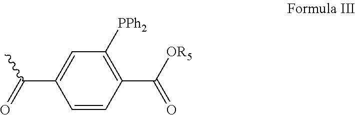

wherein R₅ is selected from H, C₁₋₂₀ alkyl, C₂₋₂₀ alkenyl, C₂₋₂₀ alkynyl, 6-20 membered aryl, 7-20 membered alkylaryl, 3-20 membered cycloalkyl, C₁₋₂₀ acyl, C₁₋₂₀ alkoxy, 7-20 membered aryloxy, C₁₋₂₀ alkylamino, C₂₋₂₀ dialkylamino, halogen, or amino, to form a first precursor; reacting the second payload with a compound according to Formula IV:

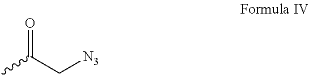

to form a second precursor; and coupling the first precursor and the second precursor to form a compound according to Formula V:

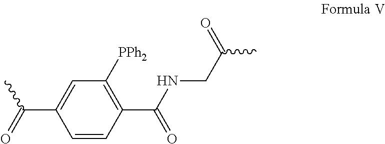

Also provided herein is a compound according to Formula II:

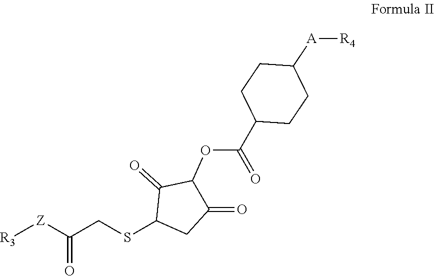

wherein R₃ and R₄ are selected in combination such that one of R₃ and R₄ comprises an antibody and the other of R₃ and R₄ comprises a drug; wherein Z is selected from —O—, —S—, and —NH—; and wherein A is selected from —O—, —S—, and —NH—.

Further provided herein is a method of covalently linking a first payload to a second payload, the method comprising: reacting the first payload with a compound according to Formula VI:

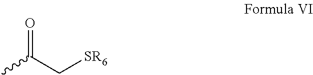

wherein R₆ is selected from is selected from H, C₁₋₂₀ alkyl, C₂₋₂₀ alkenyl, C₂₋₂₀ alkynyl, 6-20 membered aryl, 7-20 membered alkylaryl, 3-20 membered cycloalkyl, C₁₋₂₀ acyl, C₁₋₂₀ alkoxy, 7-20 membered aryloxy, C₁₋₂₀ alkylamino, C₂₋₂₀ dialkylamino, halogen, or amino, to form a first precursor; reacting the second payload with a compound according to Formula VII:

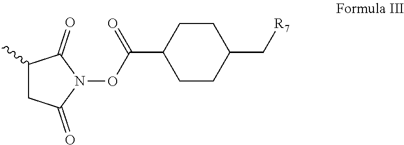

wherein R₇ is selected from is selected from H, C₁₋₂₀ alkyl, C₂₋₂₀ alkenyl, C₂₋₂₀ alkynyl, 6-20 membered aryl, 7-20 membered alkylaryl, 3-20 membered cycloalkyl, C₁₋₂₀ acyl, C₁₋₂₀ alkoxy, 7-20 membered aryloxy, C₁₋₂₀ alkylamino, C₂₋₂₀ dialkylamino, halogen, or amino, to form a second precursor; and coupling the first precursor and the second precursor to form a compound according to Formula VIII:

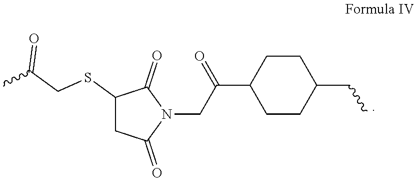

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying figures, which are incorporated in and constitute a part of this specification, illustrate several aspects described below.

FIG. 1 shows a synergetic mechanism of anti-CD276 mAb-MMAF-TLR 7/8 agonist to treat TNBCs. Step 1 is dualADC targeting and binding to the surface receptor CD276 of TNBC cells. Step 2 is internalization. Step 3 is drug release. Step 4 is MMAF-induced inhibition of microtubule polymerization. Step 5 is TLR 7/8 agonist-induced cytotoxicity. Step 6 is immune cell activation by anti-CD276 mAb and TLR 7/8 agonist.

FIG. 2 shows a synthetic pathway for constructing an example dual payload antibody-drug conjugate comprising a chemotherapy drug, an immunotherapy drug, DBM linker, and a phosphine-azide linker.

FIGS. 3A-3E show construction of DualADC via cysteine and lysine. FIG. 3A shows structure of DualADC CD276 mAb-MMAF/IMQ. FIG. 3B shows structure of DaulADC. FIG. 3C shows HPLC confirmed the conjugation of single payload and dual payloads with chimeric anti-CD276 mAb. FIG. 3D shows MALDI-TOF MS to validate the right molecular weight of mAb, single ADC (mAb-MMAF, mAb-IMQ) and DualADC (mAb-MMAF/IMQ). FIG. 3E shows SDS-PAGE of ADCs. M: Marker; 1: CD276 mAb; 2: mAb-MMAF; 3: mAb-IMQ; 4: mAb-MMAF/IMQ.

FIGS. 4A-4F show in vitro evaluations of DualADC using humanized CD276 mAb. The TNBC MDA-MB-231, MDA-MB-468 and 4T1 cells are used in cytotoxicity studies with free drugs and single-payload ADC as controls. FIG. 4A shows anti-TNBC cytotoxicity and IC₅₀ of free DM1 and MMAF drugs. FIG. 4B shows cytotoxicity and IC₅₀ of IMQ. FIG. 4C shows EC₅₀ of IMQ on human and murine TLR8⁺ HEK cells. FIG. 4D shows anti-TNBC cytotoxicity and IC₅₀ of single-payload ADC (mAb-MMAF). FIG. 4E shows cytotoxicity and IC₅₀ of single-payload (mAb-IMQ). FIG. 4F cytotoxicity and IC₅₀ of DualADC mAb-MMAF/IMQ.

FIGS. 5A-5D show anti-tumor efficacy of DualADC in TNBC PDX xenograft model. FIG. 5A shows tumor volume changes post treatment with humanized CD276 mAb-derived ADC following the schedule of Q7Dx3 as indicated by the black arrow. Data were presented as mean±SEM, n=5-7. Saline (◯), 16 mg/kg of mAb-MMAF (▴), and 16 mg/kg of mAb-MMAF/IMQ (●). *P<0.05 vs. saline using ANOVA followed by Dunnett's t-test. FIG. 5B shows body weight. FIG. 5C shows white light images at 14 days after treatment stopped. FIG. 5D shows IHC staining of TNBC PDX tumor tissue. The scale bar equals 20 μm.

FIGS. 6A-6E shows anti-TNBC efficacy of 276 mAb-MMAF/IMQ in immunocompetent models. The mouse TNBC 4T1-FLuc xenografted female BALB/cJ mice were treated with DualADC (8, 16, 24 mg/kg mAb-MMAF/IMQ), mAb-MMAF, mAb-IMQ, or saline (controls) via i.v. injection through tail vein. n=5-8. FIG. 6A shows tumor volume post treatment following schedule of Q7Dx4 as indicated by black arrow. Data were presented as mean±SEM. *P<0.05 vs. saline using ANOVA followed by Dunnett's t-test. FIG. 6B shows weight of terminal wet tumors treated with 16 mg/kg of DualADCs using chimeric CD276 mAb and humanized 276 mAb. FIG. 6C shows H&E staining of major organs. Scale bar equals 70 μm. FIG. 6D shows IHC staining of harvested tumors using markers of cell proliferation (Ki67), apoptosis (CCasp3), immune checkpoint inhibition (PD-1), and filtration and activation of CD8+T, NK and macrophage cells (CD8, CD45, F4/80). Scale bar equals 20 μm. FIG. 6E shows HE staining to analyze TNBC cell death in treatment group. Scale bar equals 40 μm.

FIGS. 7A-7B show analysis of tumoral cytokines and general toxicity post treatment of dual-payload ADC. The same mice as in FIG. 6 were used here. (FIG. 7A) Luminex assay identified several enhanced cytokines and downregulated PD-1 in TME. (FIG. 7B) The complete blood cell counts. 1: Saline (control); 2:8 mg/kg mAb-MMAF (control); 3:8 mg/kg mAb-IMQ (control); 4:8 mg/kg mAb-MMAF/IMQ; 5:16 mg/kg mAb-MMAF/IMQ; 6:24 mg/kg mAb-MMAF/IMQ.

FIGS. 8A-8D show analysis of immune cell infiltration and immune functions in TME using single-cell RNA sequencing (scRNA-Seq). The tumor tissues were harvested from the same animal study in FIG. 6. FIG. 8A shows overview of all cell types in TNBC tumors. FIG. 8B shows immune functions in TME. FIG. 8C shows immune responses of macrophage. FIG. 8D shows analysis of mitotic activities.

FIG. 9A shows a first portion of a synthetic pathway for constructing an example dual payload antibody-drug conjugate comprising a chemotherapy drug, an immunotherapy drug, DBM linker, and a SATA sulfo-SMCC linker. FIG. 9B shows a second portion of the synthetic pathway of FIG. 9A. FIG. 9C shows a third portion of the synthetic pathway of FIG. 9A. FIG. 9D shows a fourth portion of the synthetic pathway of FIG. 9A, illustrating a completed example dual payload antibody-drug conjugate.

FIGS. 10A-10D show development and engineering of CD276 mAb. FIG. 10A shows murine anti-human CD276 mAb development using hybridoma technology. FIG. 10B shows structures of murine, chimeric, and humanized anti-human CD276 mAb. FIG. 10C shows production of humanized anti-CD276 mAb using CHO cells. Dynamis medium fed with glucose, L-glutamine and Feed C. 30-mL culture in 125-mL shaker flask at 130 rpm, 5% CO₂, and 37° C. FIG. 10D shows flow cytometry to compare the surface binding of engineered CD276 mAb to normal breast cells and TNBC cells.

FIGS. 11A-11C show evaluations of TNBC targeting and internalization of chimeric anti-CD276 mAb in vitro and in vivo. FIG. 11A shows confocal imaging to test surface binding at 12 hrs after incubation and internalization of mAb-Cy5.5 (red) with MDA-MB-468 (green). FIGS. 11B-11C show live-animal IVIS to confirm mouse and human TNBC-targeting by CD276 mAb in vivo at 24 hrs post tail vein injection of fluorescent dye Cy5.5-labelled mAb (40 or 50 μg), followed with scarification, tumor and organs harvest and ex vivo imaging.

FIGS. 12A-12D show in vitro cytotoxicity of free TLR agonists and chimeric CD276 mAb-directed ADCs. Ag #2 is the IMQ used in this study. FIG. 12A shows cytotoxicity assay and IC₅₀ values of various free TLR 7/8 agonists on MDA-MB-468. FIG. 12B shows cytotoxicity assay and IC₅₀ values of various free TLR 7/8 agonists on 4T1. FIG. 12C shows cytotoxicity and IC₅₀ of chimeric anti-CD276 mAb-conjugated single-payload ADC (ChimAb-MMAF). FIG. 12D shows cytotoxicity and IC₅₀ of chimeric anti-CD276 mAb-based dual-payload ADC (ChimAb-MMAF/IMQ). Data are presented as average±STDEV. n=3. MTT assay: seeding density of 3,000-5,000 cells/well (MDA-MB-468 and MD-MB-231) or 1,000 cells/well (4T1), five-day treatment, 37° C., 5% CO₂, 96-well plate, and relative viability detected with MTT assay kit.

FIGS. 13A-13B show profiles of body weight of immunocompetent models treated with DualADC. The 4T1-FLuc xenografted female BALB/cJ mice were treated with dual-payload ADC (8, 16, 24 mg/kg mAb-MMAF/IMQ), mAb-MMAF, mAb-IMQ, or saline (controls) via i.v. injection through tail vein. n=6-8.

FIG. 14 shows additional tumoral cytokine data collected using Luminex assay with customer designed biomarker standards. The 4T1-FLuc xenografted female BALB/cJ mice were treated with saline (control) and dual-payload ADC (24 mg/kg mAb-MMAF/IMQ). n=6-8.

FIGS. 15A-15C show anti-TNBC efficacy of dual-payload ADC (chimeric CD276 mAb-MMAF/IMQ) in immunocompromised models. The MDA-MB-231-FLuc xenografted female NSG mice were treated with dual-payload ADC (16 mg/kg mAb-MMAF/IMQ), single-payload ADC (16 mg/kg mAb-MMAF), and saline (control) via i.v. injection through the tail vein. n=5. FIG. 15A shows tumor volume post treatment following schedule of Q5Dx5 as indicated by the black arrows. Tumor volume was measured with calipers and calculated as ellipsoid. Data were presented as mean±SEM. *P<0.05 vs. saline using ANOVA followed by Dunnett's t-test. FIG. 15B shows profiles of body weight change. FIG. 15C shows IVIS bioluminescence and white light images at 14 days after the final treatment injection.

FIGS. 16A-16B show pharmacokinetics (PK) study. Anti-CD276 mAb-MMAF/IMQ in BALB/cJ mouse models. 6 dosages, n=2, total of 12 mice. FIG. 16A shows serum titer of DualADC. FIG. 16B shows PK parameters.

FIGS. 17A and 17B show construction of an example Dual ADC via cysteine and lysine. FIG. 17A shows an example chemical structure of a DualADC comprising a DBM linker and a sulfo-SMCC linker. FIG. 17B shows a corresponding schematic of the DualADC.

FIGS. 18A-18H show construction of single-payload ADC via lysine or cysteine. FIG. 18A shows mAb-MMAF conjugate via lysine using bridging linker of DBM. FIG. 18B shows mAb-DM1 conjugate via lysine using linker of Sulfo-SMCC. FIG. 18C shows mAb-TLR agonist conjugate via lysine using linker of Sulfo-SMCC and SATA modification. FIG. 18D shows mAb-TLR agonist conjugate via synthesized linker of phosphine-azide. FIGS. 18E-18H are ADC (and mAb) characterizations using HPLC corresponding to FIGS. 18A-18D, respectively.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise. As used herein, the term “substantially free,” when used in the context of a composition or component of a composition that is substantially absent, is intended to refer to an amount that is then about 1% by weight or less, e.g., less than about 0.5% by weight, less than about 0.1% by weight, less than about 0.05% by weight, or less than about 0.01% by weight of the stated material, based on the total weight of the composition.

The term “subject” preferably refers to a human in need of treatment with an anti-cancer agent or treatment for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an anti-cancer agent or treatment.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cn-Cm preceding a group or moiety indicates, in each case, the possible number of carbon atoms in the group or moiety that follows. As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹.” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acetal, acyl, alkyl, alkoxy, alkenyl, alkynyl, aryl, heteroaryl, aldehyde, amino, cyano, carboxylic acid, ester, ether, carbonate ester, carbamate ester, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-1-methyl-3-pentenyl, 2-methyl-3-pentenyl, 3-methyl-3-pentenyl, 4-methyl-2-pentenyl, pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyl refers to a group with the structure —CH—CH═CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄)alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “halide” or “halogen” or “halo” as used herein refers to fluorine, chlorine, bromine, and iodine.

The terms “amine” or “amino” as used herein are represented by the formula NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula-C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “ester” as used herein is represented by the formula —OC(O)Z¹ or —C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Compositions

### Antibody-Drug Conjugate

Also provided herein is an antibody-drug comprising a monoclonal antibody (mAb), a first payload conjugated to the mAb via a first bivalent linker, and a second payload conjugated to the mAb via a second bivalent linker; wherein the first bivalent linker, second bivalent linker, or any combination thereof, comprise a phosphine-azide linker, a N-succinimidyl S-acetylthioacetate sulfo-succinimidyl-4-(N-maleimidomethyl)cyclohexane-1-carboxylate (SATA sulfo-SMCC) linker, a sulfo-succinimidyl-4-(N-maleimidomethyl)cyclohexane-1-carboxylate (sulfo-SMCC) linker, or a dibromomaleimide (DBM) linker.

In some examples, a DBM linker is a compound comprising a dibromomaleimide and a carboxylic acid connected by a carbon chain having from 2 to 13 substituted or unsubstituted carbons.

In further examples, a SATA sulfo-SMCC linker comprises a sulhydryl moiety, a succinimidyl moiety, an acetate moiety, and a cyclohexane moiety.

In certain examples, a sulfo-SMCC linker comprises a succinimidyl moiety, an acetate moiety, and a cyclohexane moiety.

In specific examples, a phosphine-azide linker comprises an azide moiety and a triphenyl phosphine moiety.

In some examples, the first bivalent linker comprises the phosphine-azide linker or the SATA sulfo-SMCC linker.

In further examples, the first bivalent linker comprises the DBM linker or the sulfo-SMCC linker.

In certain examples, the second bivalent linker comprises the phosphine-azide linker or the SATA sulfo-SMCC linker.

In specific examples, the second bivalent linker comprises the DBM linker or the sulfo-SMCC linker.

In some examples, the first bivalent linker comprises the DBM linker, and the second bivalent linker comprises any one of the sulfo-SMCC linker, the SATA sulfo-SMCC linker, or the phosphine-azide linker.

In further examples, the first bivalent linker is the DBM linker, and the second bivalent linker is the phosphine-azide linker.

In certain examples, the first bivalent linker is the DBM linker, and the second bivalent linker is the sulfo-SMCC linker.

In specific examples, the first bivalent linker is the DBM linker, and the second bivalent linker is the SATA sulfo-SMCC linker.

In some examples, the first bivalent linker covalently links the first payload to a cysteine residue present in the mAb.

In certain examples, the first bivalent linker covalently links the first payload to a lysine residue present in the mAb.

In some examples, the second bivalent linker covalently links the second payload to a cysteine residue present in the mAb

In further examples, the second bivalent linker covalently links the second payload to a lysine residue present in the mAb.

Lysine is an amino acid that is a precursor to many proteins. It contains an alpha-amino group, an alpha-carboxylic acid group, and a side chain lysyl. It is encoded by the codons AAA and AAG. The alpha-carbon is chiral, and lysine may refer to either enantiomer or a racemic mixture of both.

Cysteine is a proteinogenic amino acid with the formula HOOC—CH(—NH₂)—CH₂—SH. The thiol side chain in cysteine can participate in enzymatic reactions as a nucleophile. Cysteine is chiral and encoded by the codons UGU and UGC.

In certain examples, cysteine and/or lysine are present on the monoclonal antibody and act as binding sites for the linkers to conjugate the mAb to the first and/or second payload.

In some examples, the mAb is a murine antibody, a chimeric antibody, or a humanized antibody.

In some examples, the mAb inhibits CD276 or SSTR2.

In further examples, the mAb inhibits CD276. CD-276 (Cluster of Differentiation 276; B7-H3) is a human protein encoded by the CD276 gene. CD-276 is a 316 amino acid-long type I transmembrane protein.

In some examples, the mAb inhibits SSTR2 (Somatostatin receptor2). SSTR2 is a protein that in humans is encoded by the SSTR2 gene. The SSTR2 gene is located on chromosome 17 on the long arm in position 25.1 in humans.

In certain examples, the first payload comprises a chemotherapy drug. Chemotherapy drugs are cytotoxic by means of interfering with cell division (mitosis). Chemotherapy drugs are a means via which to damage or stress the cancerous cells in a subject. Chemotherapy drugs comprise alkylating agents, antimetabolites, anti-microtubule agents, topoisomerase inhibitors, antineoplastic agents, and cytotoxic antibiotics.

In specific examples, the chemotherapy drug comprises an antineoplastic drug. An antineoplastic agent is an agent that controls or kills cancer cells. Antineoplastic drugs are cytotoxic and are generally more damaging to dividing cells than resting cells.

In further examples, the antineoplastic drug comprises monomethyl auristatin E (MMAE) or monomethyl auristatin F (MMAF).

In some examples, the chemotherapy drug comprises Altretamine, Bendamustine, Busulfan, Carmustine, Chlorambucil, Cyclophosphamide, Dacarbazine, Ifosfamide, Lomustine, Lurbinectedin, Mechlorethamine, Melphalan, Procarbazine, Streptozocin, Temozolomide, Thiotepa, Trabectedin, Carboplatin, Cisplatin, Oxaliplatin, Bleomycin, Dactinomycin, Daunorubicin, Doxorubicin, Epirubicin, Idarubicin, Mitomycin, Mitoxantrone, Plicamycin, Valrubicin, Methotrexate, Pemetrexed, Pralatrexate, Trimetrexate, Azathioprine, Cladribine, Fludarabine, Mercaptopurine, Thioguanine, Azacitidine, Capecitabine, Cytarabine, Decitabine, Floxuridine, Fluorouracil, Gemcitabine, Trifluridine/Tipracil, Aldesleukin (IL-2), Denileukin Diftitox, Interferon Gamma, Belinostat, Panobinostat, Romidepsin, Vorinostat, Abiraterone, Apalutamide, Bicalutamide, Cyproterone, Enzalutamide, Flutamide, Nilutamide, Anastrozole, Exemestane, Fulvestrant, Letrozole, Raloxifene, Tamoxifen, Toremifene, Degarelix, Goserelin, Histrelin, Leuprolide, Relugolix, Triptorelin, Lanreotide, Octreotide, Pasireotide, Alemtuzumab, Atezolizumab, Avelumab, Belantamab, Bevacizumab, Blinatumomab, Brentuximab, Cemiplimab, Cetuximab, Daratumumab, Dinutuximab, Dostarlimab, Durvalumab, Elotuzumab, Enfortumab, Gemtuzumab, Inotuzumab Ozogamicin, Ipilimumab, Mogamulizumab, Moxetumomab Pasudotox, Necitumumab, Nivolumab, Ofatumumab, Olaratumab, Panitumumab, Pembrolizumab, Pertuzumab, Polatuzumab Vedotin, Ramucirumab, Rituximab, Sacituzumab Govitecan, Teclistamab, Tisotumab Vedotin, Tositumomab, Trastuzumab, Trastuzumab Deruxtecam, Trastuzumab Emtansine, Tremelimumab, Abemaciclib, Acalabrutinib, Adagrasib, Afatinib, Alectinib, Alpelisib, Asciminib, Axitinib, Binimetinib, Bortezomib, Bosutinib, Brigatinib, Cabozantinib, Carfilzomib, Ceritinib, Cobimetinib, Copanlisib, Crizotinib, Dabrafenib, Dacomitinib, Dasatinib, Duvelisib, Enasidenib, Encorafenib, Entrectinib, Erdafitinib, Erlotinib, Fedratinib, Futibatinib, Gefitinib, Gilteritinib, Glasdegib, Ibrutinib, Idelalisib, Imatinib, Infigratinib, Ivosidenib, Ixazomib, Lapatinib, Larotrectinib, Lenvatinib, Lorlatinib, Midostaurin, Mobocertinib, Momelotinib, Neratinib, Nilotinib, Niraparib, Olaparib, Olutasidenib, Osimertinib, Pacritinib, Palbociclib, Pazopanib, Pemigatinib, Pexidartinib, Ponatinib, Regorafenib, Ribocicib, Ripretinib, Rucaparib, Ruxolitinib, Selumetinib, Sonidegib, Sorafenib, Sunitinib, Talazoparib, Tivozanib, Trametinib, Trilaciblib, Umbralisib, Vandetanib, Vemurafenib, Vismodegib, Zanubrutinib, Cabazitaxel, Docetaxel, Paclitaxel, Etoposide, Irinotecan, Teniposide, Topotecan, Vinblastine, Vincristine, Vinorelbine, Asparaginase (Pegaspargase), Belzutifan, Bexarotene, Cedazuridine, Eribulin, Everolimus, Hydroxyurea, Ixabepilone, Lenalidomide, Mitotane, Omacetaxine, Pomalidomide, Selinexor, Tagraxofusp, Tazemetostat, Tebentafusp, Telotristat, Temsirolimus, Thalidomide, Venetoclax, or any combination thereof.

In further examples, the second payload comprises an immunotherapy drug. Immunotherapy drug is a type of cancer treatment that helps a subject's immune system fight cancer. It is a type of biological therapy, using substances made from living organisms. Immunotherapy types include immune checkpoint inhibitors, T-cell transfer therapy, monoclonal antibodies, treatment vaccines, and immune system modulators.

In some examples, the immunotherapy drug comprises a toll like receptor agonist. Toll like receptors are a class of proteins that play a key role in the innate immune system. They are single-spanning receptors and include TLR1, TLR2, TLR3, TLR4, TLR5, TLR6, TLR7, TLR8, TLR9, TLR10, TLR11, TLR12, and TLR13. Humans lack gees for TLR11, TLR12, and TLR13 and mice lack a functional gene for TLR10. The receptors TLE1, TLR2, TLR4, TLR5, TLR6, and TLR10 are located on the cell membrane and TLR3, TLR7, TLR8, and TLR9 are located in intracellular vesicles.

In certain examples, the immunotherapy drug comprises a toll like receptor 7 or 8 (TLR 7/8) agonist. TLR7/8 agonists stimulate the innate immune system to harness an anti-CTCL effect by the production of cytokines. TLRs recognize microbes by binding to pathogen-associated molecular patterns. This binding activates immunity and inflammatory cascades.

In specific examples, the TLR 7/8 agonist comprises imidazoquinoline. Imidazoquinolines is a tricyclic organic molecule and includes imiquimod, gardiquimod, and resiquimod.

In some examples, the immunotherapy drug comprises brexucabtagene autoleucel, trastuzumab, aldesleukin, amivantamab, atezolizumab, avelumab, axicabtagene ciloleucel, belantamab mafodotin, bevacizumab, blinatumomab, brentuximab vedotin, cemiplimab, cetuximab, ciltacabtagene, daratumumab, daratumumab, dostarlimab, durvalumab, elotuzumab, enfortumab vedotin, epcoritamab, gemtuzumab, glofitamab, ibritumomab tioxetan, idecabtagene vicleucel, inotuzumab ozogamicin, ipilimumab, isatuximab, lisocabtagene maraleucel, loncastuximab tesirine, margetuximab, mirvetuximab soravtansine, mogamulizumab, mosunetuzumab, nadofaragene firadenovec, naxitamamb, necitumumab, nivolumab, nivolumab and relatlimab, obinutuzumab, ofatumumab, panitumumab, peginterferon, pembrolizumab, pertuzumab, polatuzumab vedotin, ramucirumab, retifanlimab, rituximab, Sacituzumab govitecan, siltuximab, sipuleucel-T, tafasitamamb, tagraxofusp, talimogene laherparepvec, talquetamab, tebentafusp, teclistamab, tisagenlecleucel, tisotumab vedotin, trastuzumab, tremelimumab, aflibercept, or any combination thereof.

Further provided herein is an antibody-drug conjugate comprising a monoclonal antibody (mAb) and a payload conjugated to the mAb via a bivalent linker, wherein the bivalent linker comprises a phosphine-azide linker or a N-succinimidyl S-acetylthioacetate sulfo-succinimidyl-4-(N-maleimidomethyl)cyclohexane-1-carboxylate (sulfo-SMCC) (SATA sulfo-SMCC) linker.

In some examples, the bivalent linker comprises the phosphine-azide linker.

In further examples, the bivalent linker comprises the SATA sulfo-SMCC linker.

In certain examples, the bivalent linker covalently links the payload to a cysteine residue present in the mAb.

In specific examples, the bivalent linker covalently links the payload to a lysine residue present in the mAb.

In some examples, the mAb is a murine antibody, a chimeric antibody, or a humanized antibody.

In some examples, the mAb inhibits CD276 or SSTR2.

In further examples, the mAb inhibits CD276.

In specific examples, the mAb inhibits SSTR2.

In certain examples, the payload comprises a chemotherapy drug.

In specific examples, the chemotherapy drug comprises an antineoplastic drug.

In further examples, the antineoplastic drug comprises MMAE or MMAF.

In some examples, the chemotherapy drug comprises Altretamine, Bendamustine, Busulfan, Carmustine, Chlorambucil, Cyclophosphamide, Dacarbazine, Ifosfamide, Lomustine, Lurbinectedin, Mechlorethamine, Melphalan, Procarbazine, Streptozocin, Temozolomide, Thiotepa, Trabectedin, Carboplatin, Cisplatin, Oxaliplatin, Bleomycin, Dactinomycin, Daunorubicin, Doxorubicin, Epirubicin, Idarubicin, Mitomycin, Mitoxantrone, Plicamycin, Valrubicin, Methotrexate, Pemetrexed, Pralatrexate, Trimetrexate, Azathioprine, Cladribine, Fludarabine, Mercaptopurine, Thioguanine, Azacitidine, Capecitabine, Cytarabine, Decitabine, Floxuridine, Fluorouracil, Gemcitabine, Trifluridine/Tipracil, Aldesleukin (IL-2), Denileukin Diftitox, Interferon Gamma, Belinostat, Panobinostat, Romidepsin, Vorinostat, Abiraterone, Apalutamide, Bicalutamide, Cyproterone, Enzalutamide, Flutamide, Nilutamide, Anastrozole, Exemestane, Fulvestrant, Letrozole, Raloxifene, Tamoxifen, Toremifene, Degarelix, Goserelin, Histrelin, Leuprolide, Relugolix, Triptorelin, Lanreotide, Octreotide, Pasireotide, Alemtuzumab, Atezolizumab, Avelumab, Belantamab, Bevacizumab, Blinatumomab, Brentuximab, Cemiplimab, Cetuximab, Daratumumab, Dinutuximab, Dostarlimab, Durvalumab, Elotuzumab, Enfortumab, Gemtuzumab, Inotuzumab Ozogamicin, Ipilimumab, Mogamulizumab, Moxetumomab Pasudotox, Necitumumab, Nivolumab, Ofatumumab, Olaratumab, Panitumumab, Pembrolizumab, Pertuzumab, Polatuzumab Vedotin, Ramucirumab, Rituximab, Sacituzumab Govitecan, Teclistamab, Tisotumab Vedotin, Tositumomab, Trastuzumab, Trastuzumab Deruxtecam, Trastuzumab Emtansine, Tremelimumab, Abemaciclib, Acalabrutinib, Adagrasib, Afatinib, Alectinib, Alpelisib, Asciminib, Axitinib, Binimetinib, Bortezomib, Bosutinib, Brigatinib, Cabozantinib, Carfilzomib, Ceritinib, Cobimetinib, Copanlisib, Crizotinib, Dabrafenib, Dacomitinib, Dasatinib, Duvelisib, Enasidenib, Encorafenib, Entrectinib, Erdafitinib, Erlotinib, Fedratinib, Futibatinib, Gefitinib, Gilteritinib, Glasdegib, Ibrutinib, Idelalisib, Imatinib, Infigratinib, Ivosidenib, Ixazomib, Lapatinib, Larotrectinib, Lenvatinib, Lorlatinib, Midostaurin, Mobocertinib, Momelotinib, Neratinib, Nilotinib, Niraparib, Olaparib, Olutasidenib, Osimertinib, Pacritinib, Palbociclib, Pazopanib, Pemigatinib, Pexidartinib, Ponatinib, Regorafenib, Ribocicib, Ripretinib, Rucaparib, Ruxolitinib, Selumetinib, Sonidegib, Sorafenib, Sunitinib, Talazoparib, Tivozanib, Trametinib, Trilaciblib, Umbralisib, Vandetanib, Vemurafenib, Vismodegib, Zanubrutinib, Cabazitaxel, Docetaxel, Paclitaxel, Etoposide, Irinotecan, Teniposide, Topotecan, Vinblastine, Vincristine, Vinorelbine, Asparaginase (Pegaspargase), Belzutifan, Bexarotene, Cedazuridine, Eribulin, Everolimus, Hydroxyurea, Ixabepilone, Lenalidomide, Mitotane, Omacetaxine, Pomalidomide, Selinexor, Tagraxofusp, Tazemetostat, Tebentafusp, Telotristat, Temsirolimus, Thalidomide, Venetoclax, or any combination thereof.

In further examples, the payload comprises an immunotherapy drug.

In some examples, the immunotherapy drug comprises a toll like receptor agonist, such as a toll like receptor 7 or 8 (TLR 7/8) agonist.

In specific examples, the TLR 7/8 agonist comprises an imidazoquinoline.

In some examples, the immunotherapy drug comprises brexucabtagene autoleucel, trastuzumab, aldesleukin, amivantamab, atezolizumab, avelumab, axicabtagene ciloleucel, belantamab mafodotin, bevacizumab, blinatumomab, brentuximab vedotin, cemiplimab, cetuximab, ciltacabtagene, daratumumab, daratumumab, dostarlimab, durvalumab, elotuzumab, enfortumab vedotin, epcoritamab, gemtuzumab, glofitamab, ibritumomab tioxetan, idecabtagene vicleucel, inotuzumab ozogamicin, ipilimumab, isatuximab, lisocabtagene maraleucel, loncastuximab tesirine, margetuximab, mirvetuximab soravtansine, mogamulizumab, mosunetuzumab, nadofaragene firadenovec, naxitamamb, necitumumab, nivolumab, nivolumab and relatlimab, obinutuzumab, ofatumumab, panitumumab, peginterferon, pembrolizumab, pertuzumab, polatuzumab vedotin, ramucirumab, retifanlimab, rituximab, Sacituzumab govitecan, siltuximab, sipuleucel-T, tafasitamamb, tagraxofusp, talimogene laherparepvec, talquetamab, tebentafusp, teclistamab, tisagenlecleucel, tisotumab vedotin, trastuzumab, tremelimumab, aflibercept, or any combination thereof.

### Compounds

Also provided herein is a compound according to Formula I:

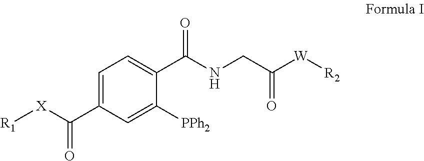

wherein R₁ and R₂ are selected in combination such that one of R₁ and R₂ comprises an antibody and the other of R₁ and R₂ comprises a drug; X is selected from —O—, —S—, and —NH—; and W is selected from —O—, —S—, and —NH—.

In some examples, X is NH.

In further examples, W is NH.

In certain examples, R₁ comprises the antibody.

In specific examples, R₂ comprises the drug.

In some examples, the antibody is a monoclonal antibody (mAb).

In further examples, the mAb is a murine antibody, a chimeric antibody, or a humanized antibody.

In some examples, the mAb inhibits CD276 or SSTR2.

In certain examples, the mAb inhibits CD276.

In some examples, the mAb inhibits SSTR2.

In specific examples, the drug is an immunotherapy drug.

In some examples, the immunotherapy drug comprises a toll like receptor agonist, such as toll like receptor 7 or 8 (TLR 7/8) agonist.

In further examples, the TLR 7/8 agonist comprises imidazoquinoline.

In certain examples, the immunotherapy drug comprises brexucabtagene autoleucel, trastuzumab, aldesleukin, amivantamab, atezolizumab, avelumab, axicabtagene ciloleucel, belantamab mafodotin, bevacizumab, blinatumomab, brentuximab vedotin, cemiplimab, cetuximab, ciltacabtagene, daratumumab, daratumumab, dostarlimab, durvalumab, elotuzumab, enfortumab vedotin, epcoritamab, gemtuzumab, glofitamab, ibritumomab tioxetan, idecabtagene vicleucel, inotuzumab ozogamicin, ipilimumab, isatuximab, lisocabtagene maraleucel, loncastuximab tesirine, margetuximab, mirvetuximab soravtansine, mogamulizumab, mosunetuzumab, nadofaragene firadenovec, naxitamamb, necitumumab, nivolumab, nivolumab and relatlimab, obinutuzumab, ofatumumab, panitumumab, peginterferon, pembrolizumab, pertuzumab, polatuzumab vedotin, ramucirumab, retifanlimab, rituximab, Sacituzumab govitecan, siltuximab, sipuleucel-T, tafasitamamb, tagraxofusp, talimogene laherparepvec, talquetamab, tebentafusp, teclistamab, tisagenlecleucel, tisotumab vedotin, trastuzumab, tremelimumab, aflibercept, or any combination thereof.

Further provided herein is a compound according to Formula II:

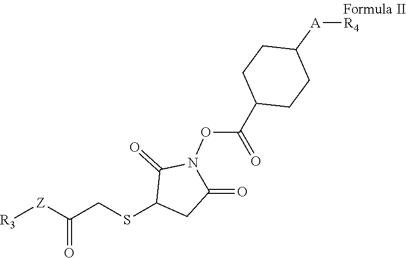

wherein R₃ and R₄ are selected in combination such that one of R₃ and R₄ comprises an antibody and the other of R₃ and R₄ comprises a drug; wherein Z is selected from —O—, —S—, and —NH—; and wherein A is selected from —O—, —S—, and —NH—.

In some examples, Z is an NH.

In further examples, A is an NH.

In certain examples, R₃ comprises the antibody.

In specific examples, R₄ comprises the drug.

In some examples, the antibody is a monoclonal antibody (mAb).

In further examples, the mAb is a murine antibody, a chimeric antibody, or a humanized antibody.

In some examples, the mAb inhibits CD276 or SSTR2.

In certain examples, the mAb inhibits CD276.

In some examples, the mAb inhibits SSTR2.

In specific examples, the drug is an immunotherapy drug.

In some examples, the immunotherapy drug comprises a toll like receptor agonist, such as toll like receptor 7 or 8 (TLR 7/8) agonist.

In further examples, the TLR 7/8 agonist comprises an imidazoquinoline.

In certain examples, the immunotherapy drug comprises brexucabtagene autoleucel, trastuzumab, aldesleukin, amivantamab, atezolizumab, avelumab, axicabtagene ciloleucel, belantamab mafodotin, bevacizumab, blinatumomab, brentuximab vedotin, cemiplimab, cetuximab, ciltacabtagene, daratumumab, daratumumab, dostarlimab, durvalumab, elotuzumab, enfortumab vedotin, epcoritamab, gemtuzumab, glofitamab, ibritumomab tioxetan, idecabtagene vicleucel, inotuzumab ozogamicin, ipilimumab, isatuximab, lisocabtagene maraleucel, loncastuximab tesirine, margetuximab, mirvetuximab soravtansine, mogamulizumab, mosunetuzumab, nadofaragene firadenovec, naxitamamb, necitumumab, nivolumab, nivolumab and relatlimab, obinutuzumab, ofatumumab, panitumumab, peginterferon, pembrolizumab, pertuzumab, polatuzumab vedotin, ramucirumab, retifanlimab, rituximab, Sacituzumab govitecan, siltuximab, sipuleucel-T, tafasitamamb, tagraxofusp, talimogene laherparepvec, talquetamab, tebentafusp, teclistamab, tisagenlecleucel, tisotumab vedotin, trastuzumab, tremelimumab, aflibercept, or any combination thereof.

### Method

### Method of Treating a Disease

The present disclosure, in one aspect, provides for a method of treating a disease in a subject in need thereof comprising administering to the subject a therapeutically effective amount of the antibody-drug conjugate as disclosed herein.

In some examples, the disease is cancer. In further examples, the cancer is triple negative breast cancer (TNBC), glioblastoma (GBM), or non-small cell lung cancer (NSCLC). Triple-negative breast cancer (TNBC) is an aggressive type of invasive breast cancer and accounts for 15% of all breast cancer cases. TNBC is estrogen receptor-negative and progesterone receptor-negative, as well as HER2-negative. TNBC is often more aggressive than other breast cancers, as well as harder to treat and more likely to recur than cancers that are hormone receptor-positive or HER2-positive.

Glioblastoma (GBM) is a cancer that starts as a growth of cells in the brain or spinal cord. It forms from cells called astrocytes, which support nerve cells. Symptoms of glioblastoma may include headaches, nausea and vomiting, blurred or double vision, trouble speaking, altered sense of touch, and seizures. Glioblastomas are malignant grade 4 tumors that are predominantly made of abnormal astrocytic cells. GBMs invade regions of the brain, most commonly the frontal lobe, and can spread to the opposite side of the brain through connection fibers (corpus callosum) or the ventricular system.

Non-small cell lung cancer (NSCLC) is a common type of lung cancer that begins at the cellular level and causes abnormal cells in the lungs to reproduce rapidly and out of control. NSCLCs include carcinomas (e.g., adenocarcinoma, squamous cell carcinoma, large cell carcinoma), which are cancers of the cells lining the surface of the lung airways, such as bronchi, bronchioles, and alveoli. Symptoms of NSCLC include a persistent cough, coughing up blood, chest pain or discomfort, trouble breathing, wheezing, hoarseness, loss of appetite, weight loss for no reason, fatigue, trouble swallowing, swelling in the face and/or veins in the neck, or any combination thereof.

### Method of Upregulating Tumoral Immunity

Also provided herein is a method of upregulating tumoral immunity of a subject in need thereof comprising administering to the subject a therapeutically effective amount of the antibody-drug conjugate as disclosed herein.

Tumoral immunity refers to innate and adaptive immune responses which lead to tumor control.

### Method of Reducing Growth of a Tumor

Further provided herein is a method of reducing growth of a tumor in a subject in need thereof, comprising administering to the subject a therapeutically effective amount of the antibody-drug conjugate as disclosed herein.

In some examples, the tumor is a squamous cell carcinoma, large cell carcinoma, adenocarcinoma, invasive ductal carcinoma, ductal carcinoma in situ, adenoid cystic carcinoma, mucoepidermoid carcinoma, glioblastoma, or any combination thereof.

Squamous cell carcinoma, also known as cutaneous squamous cell carcinoma, is a malignant tumor and one of the most common types of skin cancer.

Large cell carcinoma (LCC) is a type of NSCLC that is a group of malignant neoplasms that are undifferentiated. It is the least common type of NSCLC, comprising about 10% to 15% of all NSCLC diagnoses. It can tend to grow and spread more rapidly than other forms of lung cancer.

Adenocarcinoma forms in glandular tissue, which lines certain internal organs and make and releases substances in the body, such as mucus, digestive juices, and other fluids. Most cancers of the breast, lung, esophagus, stomach, colon, rectum, pancreas, prostate, and uterus are adenocarcinomas.

Invasive ductal carcinoma is the most common form of breast cancer, accounting for 80% of all breast cancer diagnoses. Invasive ductal carcinoma occurs when abnormal cells growing in the lining of the milk ducts change and invade breast tissue beyond the walls of the duct. From there, cancer cells can spread and break into lymph nodes or the bloodstream, wherein they can travel to other organs and areas in the body, resulting in metastatic breast cancer.

Ductal carcinoma in situ is the presence of abnormal cells inside a milk duct in the breast. It is considered the earliest form of breast cancer and is considered noninvasive. It is usually found during a mammogram done as part of a breast cancer screening or to investigate a breast lump.

Adenoid cystic carcinoma is a rare malignancy arising from the secretory glands, most commonly seen in the salivary glands. This tumor is typically slow-growing compared to other carcinomas and has a tendency for perineural invasion as well as hematogenous spread to distant organs and is most commonly seen in the elderly.

Mucoepidermoid carcinomas are rare salivary gland-type tumors that comprise mucin-secreting cells, squamous cells, and intermediate-type cells. They make up a distinct group of lung malignancies accounting for less than 1% of all lung cancers.

Glioblastoma refers to the tumors resulting from the glioblastoma as discussed above.

### Method of Targeting a Protein

Also provided herein is a method of inhibiting a protein in a cell in need thereof, comprising administering to the cell a therapeutically effective amount of the antibody-drug conjugate as disclosed herein, wherein the antibody in the antibody-drug conjugate inhibits the protein.

A protein comprises one or more long, folded chains of amino acids (each called a polypeptide), wherein the protein sequences are determined by the DNA sequence of the protein-encoding gene. There are four levels of protein organization: primary protein structure, secondary protein structure, tertiary protein structure, and quaternary protein structure. Proteins in cells include antibodies, contractile proteins, enzymes, hormonal proteins, structural proteins, storage proteins, and transport proteins.

### Method of Bioconjugation

Further provided herein is a method of covalently linking a first payload to a second payload, the method comprising: reacting the first payload with a compound according to Formula III:

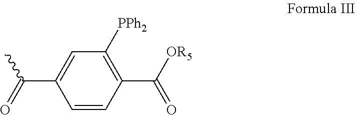

wherein R₅ is selected from H, C₁₋₂₀ alkyl, C₂₋₂₀ alkenyl, C₂₋₂₀ alkynyl, 6-20 membered aryl, 7-20 membered alkylaryl, 3-20 membered cycloalkyl, C₁₋₂₀ acyl, C₁₋₂₀ alkoxy, 7-20 membered aryloxy, C₁₋₂₀ alkylamino, C₂₋₂₀ dialkylamino, halogen, or amino, to form a first precursor; reacting the second payload with a compound according to Formula IV:

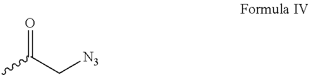

to form a second precursor; and coupling the first precursor and the second precursor to form a compound according to Formula V:

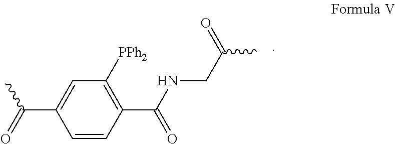

In some examples, R₅ is a C₁ alkyl.

In some examples, the first payload comprises a biomolecule. A biomolecule is a molecule relating to living organisms and includes, in some examples, large macromolecules such as proteins, carbohydrates, lipids, and nucleic acids, as well as, in further examples, small molecules such as vitamins and hormones.

In further examples, the biomolecule is a protein or an antibody.

In certain examples, the second payload is a surface, a particle, a drug, or a second biomolecule.

In specific examples, the drug is an immunotherapy drug.

In some examples, the immunotherapy drug comprises a toll like receptor agonist, such as a toll like receptor 7 or 8 (TLR 7/8) agonist.

In further examples, the TLR 7/8 agonist comprises an imidazoquinoline.

In certain examples, the immunotherapy drug comprises brexucabtagene autoleucel, trastuzumab, aldesleukin, amivantamab, atezolizumab, avelumab, axicabtagene ciloleucel, belantamab mafodotin, bevacizumab, blinatumomab, brentuximab vedotin, cemiplimab, cetuximab, ciltacabtagene, daratumumab, daratumumab, dostarlimab, durvalumab, elotuzumab, enfortumab vedotin, epcoritamab, gemtuzumab, glofitamab, ibritumomab tioxetan, idecabtagene vicleucel, inotuzumab ozogamicin, ipilimumab, isatuximab, lisocabtagene maraleucel, loncastuximab tesirine, margetuximab, mirvetuximab soravtansine, mogamulizumab, mosunetuzumab, nadofaragene firadenovec, naxitamamb, necitumumab, nivolumab, nivolumab and relatlimab, obinutuzumab, ofatumumab, panitumumab, peginterferon, pembrolizumab, pertuzumab, polatuzumab vedotin, ramucirumab, retifanlimab, rituximab, Sacituzumab govitecan, siltuximab, sipuleucel-T, tafasitamamb, tagraxofusp, talimogene laherparepvec, talquetamab, tebentafusp, teclistamab, tisagenlecleucel, tisotumab vedotin, trastuzumab, tremelimumab, aflibercept, or any combination thereof.

In specific examples, the drug is a chemotherapy drug.

In some examples, the chemotherapy drug comprises an antineoplastic drug.

In certain examples, the antineoplastic drug comprises MMAE or MMAF.

In further examples, the chemotherapy drug comprises Altretamine, Bendamustine, Busulfan, Carmustine, Chlorambucil, Cyclophosphamide, Dacarbazine, Ifosfamide, Lomustine, Lurbinectedin, Mechlorethamine, Melphalan, Procarbazine, Streptozocin, Temozolomide, Thiotepa, Trabectedin, Carboplatin, Cisplatin, Oxaliplatin, Bleomycin, Dactinomycin, Daunorubicin, Doxorubicin, Epirubicin, Idarubicin, Mitomycin, Mitoxantrone, Plicamycin, Valrubicin, Methotrexate, Pemetrexed, Pralatrexate, Trimetrexate, Azathioprine, Cladribine, Fludarabine, Mercaptopurine, Thioguanine, Azacitidine, Capecitabine, Cytarabine, Decitabine, Floxuridine, Fluorouracil, Gemcitabine, Trifluridine/Tipracil, Aldesleukin (IL-2), Denileukin Diftitox, Interferon Gamma, Belinostat, Panobinostat, Romidepsin, Vorinostat, Abiraterone, Apalutamide, Bicalutamide, Cyproterone, Enzalutamide, Flutamide, Nilutamide, Anastrozole, Exemestane, Fulvestrant, Letrozole, Raloxifene, Tamoxifen, Toremifene, Degarelix, Goserelin, Histrelin, Leuprolide, Relugolix, Triptorelin, Lanreotide, Octreotide, Pasireotide, Alemtuzumab, Atezolizumab, Avelumab, Belantamab, Bevacizumab, Blinatumomab, Brentuximab, Cemiplimab, Cetuximab, Daratumumab, Dinutuximab, Dostarlimab, Durvalumab, Elotuzumab, Enfortumab, Gemtuzumab, Inotuzumab Ozogamicin, Ipilimumab, Mogamulizumab, Moxetumomab Pasudotox, Necitumumab, Nivolumab, Ofatumumab, Olaratumab, Panitumumab, Pembrolizumab, Pertuzumab, Polatuzumab Vedotin, Ramucirumab, Rituximab, Sacituzumab Govitecan, Teclistamab, Tisotumab Vedotin, Tositumomab, Trastuzumab, Trastuzumab Deruxtecam, Trastuzumab Emtansine, Tremelimumab, Abemaciclib, Acalabrutinib, Adagrasib, Afatinib, Alectinib, Alpelisib, Asciminib, Axitinib, Binimetinib, Bortezomib, Bosutinib, Brigatinib, Cabozantinib, Carfilzomib, Ceritinib, Cobimetinib, Copanlisib, Crizotinib, Dabrafenib, Dacomitinib, Dasatinib, Duvelisib, Enasidenib, Encorafenib, Entrectinib, Erdafitinib, Erlotinib, Fedratinib, Futibatinib, Gefitinib, Gilteritinib, Glasdegib, Ibrutinib, Idelalisib, Imatinib, Infigratinib, Ivosidenib, Ixazomib, Lapatinib, Larotrectinib, Lenvatinib, Lorlatinib, Midostaurin, Mobocertinib, Momelotinib, Neratinib, Nilotinib, Niraparib, Olaparib, Olutasidenib, Osimertinib, Pacritinib, Palbociclib, Pazopanib, Pemigatinib, Pexidartinib, Ponatinib, Regorafenib, Ribocicib, Ripretinib, Rucaparib, Ruxolitinib, Selumetinib, Sonidegib, Sorafenib, Sunitinib, Talazoparib, Tivozanib, Trametinib, Trilaciblib, Umbralisib, Vandetanib, Vemurafenib, Vismodegib, Zanubrutinib, Cabazitaxel, Docetaxel, Paclitaxel, Etoposide, Irinotecan, Teniposide, Topotecan, Vinblastine, Vincristine, Vinorelbine, Asparaginase (Pegaspargase), Belzutifan, Bexarotene, Cedazuridine, Eribulin, Everolimus, Hydroxyurea, Ixabepilone, Lenalidomide, Mitotane, Omacetaxine, Pomalidomide, Selinexor, Tagraxofusp, Tazemetostat, Tebentafusp, Telotristat, Temsirolimus, Thalidomide, Venetoclax, or any combination thereof.

Also provided herein is a method of covalently linking a first payload to a second payload, the method comprising: reacting the first payload with a compound according to

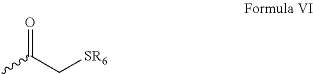

wherein R₆ is selected from is selected from H, C₁₋₂₀ alkyl, C₂₋₂₀ alkenyl, C₂₋₂₀ alkynyl, 6-20 membered aryl, 7-20 membered alkylaryl, 3-20 membered cycloalkyl, C₁₋₂₀ acyl, C₁₋₂₀ alkoxy, 7-20 membered aryloxy, C₁₋₂₀ alkylamino, C₂₋₂₀ dialkylamino, halogen, or amino, to form a first precursor; reacting the second payload with a compound according to Formula VII:

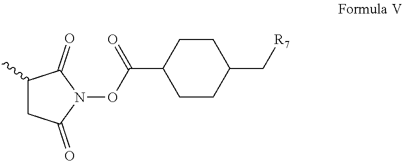

wherein R₇ is selected from is selected from H, C₁₋₂₀ alkyl, C₂₋₂₀ alkenyl, C₂₋₂₀ alkynyl, 6-20 membered aryl, 7-20 membered alkylaryl, 3-20 membered cycloalkyl, C₁₋₂₀ acyl, C₁₋₂₀ alkoxy, 7-20 membered aryloxy, C₁₋₂₀ alkylamino, C₂₋₂₀ dialkylamino, halogen, or amino, to form a second precursor; and coupling the first precursor and the second precursor to form a compound according to Formula VIII:

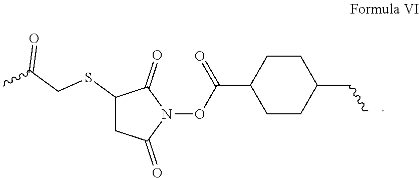

In some examples, R₆ is

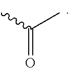

In further examples, R₇ is

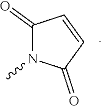

In some examples, the first payload comprises a biomolecule.

In further examples, the biomolecule is a protein or an antibody.

In certain examples, the second payload is a surface, a particle, a drug, or a second biomolecule.

In specific examples, the drug is an immunotherapy drug.

In some examples, the immunotherapy drug comprises a toll like receptor agonist, such as toll like receptor 7 or 8 (TLR 7/8) agonist.

In further examples, the TLR 7/8 agonist comprises imidazoquinoline.

In certain examples, the immunotherapy drug comprises brexucabtagene autoleucel, trastuzumab, aldesleukin, amivantamab, atezolizumab, avelumab, axicabtagene ciloleucel, belantamab mafodotin, bevacizumab, blinatumomab, brentuximab vedotin, cemiplimab, cetuximab, ciltacabtagene, daratumumab, daratumumab, dostarlimab, durvalumab, elotuzumab, enfortumab vedotin, epcoritamab, gemtuzumab, glofitamab, ibritumomab tioxetan, idecabtagene vicleucel, inotuzumab ozogamicin, ipilimumab, isatuximab, lisocabtagene maraleucel, loncastuximab tesirine, margetuximab, mirvetuximab soravtansine, mogamulizumab, mosunetuzumab, nadofaragene firadenovec, naxitamamb, necitumumab, nivolumab, nivolumab and relatlimab, obinutuzumab, ofatumumab, panitumumab, peginterferon, pembrolizumab, pertuzumab, polatuzumab vedotin, ramucirumab, retifanlimab, rituximab, Sacituzumab govitecan, siltuximab, sipuleucel-T, tafasitamamb, tagraxofusp, talimogene laherparepvec, talquetamab, tebentafusp, teclistamab, tisagenlecleucel, tisotumab vedotin, trastuzumab, tremelimumab, aflibercept, or any combination thereof.

In specific examples, the drug is a chemotherapy drug.

In some examples, the chemotherapy drug comprises an antineoplastic drug, such as MMAE or MMAF.

In further examples, the chemotherapy drug comprises Altretamine, Bendamustine, Busulfan, Carmustine, Chlorambucil, Cyclophosphamide, Dacarbazine, Ifosfamide, Lomustine, Lurbinectedin, Mechlorethamine, Melphalan, Procarbazine, Streptozocin, Temozolomide, Thiotepa, Trabectedin, Carboplatin, Cisplatin, Oxaliplatin, Bleomycin, Dactinomycin, Daunorubicin, Doxorubicin, Epirubicin, Idarubicin, Mitomycin, Mitoxantrone, Plicamycin, Valrubicin, Methotrexate, Pemetrexed, Pralatrexate, Trimetrexate, Azathioprine, Cladribine, Fludarabine, Mercaptopurine, Thioguanine, Azacitidine, Capecitabine, Cytarabine, Decitabine, Floxuridine, Fluorouracil, Gemcitabine, Trifluridine/Tipracil, Aldesleukin (IL-2), Denileukin Diftitox, Interferon Gamma, Belinostat, Panobinostat, Romidepsin, Vorinostat, Abiraterone, Apalutamide, Bicalutamide, Cyproterone, Enzalutamide, Flutamide, Nilutamide, Anastrozole, Exemestane, Fulvestrant, Letrozole, Raloxifene, Tamoxifen, Toremifene, Degarelix, Goserelin, Histrelin, Leuprolide, Relugolix, Triptorelin, Lanreotide, Octreotide, Pasireotide, Alemtuzumab, Atezolizumab, Avelumab, Belantamab, Bevacizumab, Blinatumomab, Brentuximab, Cemiplimab, Cetuximab, Daratumumab, Dinutuximab, Dostarlimab, Durvalumab, Elotuzumab, Enfortumab, Gemtuzumab, Inotuzumab Ozogamicin, Ipilimumab, Mogamulizumab, Moxetumomab Pasudotox, Necitumumab, Nivolumab, Ofatumumab, Olaratumab, Panitumumab, Pembrolizumab, Pertuzumab, Polatuzumab Vedotin, Ramucirumab, Rituximab, Sacituzumab Govitecan, Teclistamab, Tisotumab Vedotin, Tositumomab, Trastuzumab, Trastuzumab Deruxtecam, Trastuzumab Emtansine, Tremelimumab, Abemaciclib, Acalabrutinib, Adagrasib, Afatinib, Alectinib, Alpelisib, Asciminib, Axitinib, Binimetinib, Bortezomib, Bosutinib, Brigatinib, Cabozantinib, Carfilzomib, Ceritinib, Cobimetinib, Copanlisib, Crizotinib, Dabrafenib, Dacomitinib, Dasatinib, Duvelisib, Enasidenib, Encorafenib, Entrectinib, Erdafitinib, Erlotinib, Fedratinib, Futibatinib, Gefitinib, Gilteritinib, Glasdegib, Ibrutinib, Idelalisib, Imatinib, Infigratinib, Ivosidenib, Ixazomib, Lapatinib, Larotrectinib, Lenvatinib, Lorlatinib, Midostaurin, Mobocertinib, Momelotinib, Neratinib, Nilotinib, Niraparib, Olaparib, Olutasidenib, Osimertinib, Pacritinib, Palbociclib, Pazopanib, Pemigatinib, Pexidartinib, Ponatinib, Regorafenib, Ribocicib, Ripretinib, Rucaparib, Ruxolitinib, Selumetinib, Sonidegib, Sorafenib, Sunitinib, Talazoparib, Tivozanib, Trametinib, Trilaciblib, Umbralisib, Vandetanib, Vemurafenib, Vismodegib, Zanubrutinib, Cabazitaxel, Docetaxel, Paclitaxel, Etoposide, Irinotecan, Teniposide, Topotecan, Vinblastine, Vincristine, Vinorelbine, Asparaginase (Pegaspargase), Belzutifan, Bexarotene, Cedazuridine, Eribulin, Everolimus, Hydroxyurea, Ixabepilone, Lenalidomide, Mitotane, Omacetaxine, Pomalidomide, Selinexor, Tagraxofusp, Tazemetostat, Tebentafusp, Telotristat, Temsirolimus, Thalidomide, Venetoclax, or any combination thereof.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: CD276 (B7-H3)-Targeting Dual-Payload Antibody-Drug Conjugate for Chemo-Immunotherapy of Triple-Negative Breast Cancers

### Introduction

Triple-negative breast cancers (TNBCs) are highly aggressive and heterologous and often relapse post standard radiotherapies and cytotoxic chemotherapies with poor clinical benefits. Discussed herein is the development of an innovative antibody-dual payload conjugate (DualADC) as a chemo-immunotherapy of TNBCs. Specifically, the overexpression of an immune checkpoint transmembrane CD276 (B7-H3), associated with angiogenesis, metastasis and immune tolerance, in over 60% of TNBC patients was detected. A new monoclonal antibody (mAb), capable of targeting the extracellular domain of surface CD276, delivering payloads and upregulating tumoral immunity, was developed and engineered.

Furthermore, an innovative platform for concurrent conjugation of traditional cytotoxic payload and immunoregulating toll-like receptor 7/8 agonist with CD276 mAb was established. Evaluations demonstrated that this therapy effectively killed multiple TNBC subtypes, significantly enhanced immune functions in the tumor microenvironment, and reduced tumor burden by up to 90-100% in animal studies. The post-treatment analysis using single-cell RNA sequencing, Luminex multiplex cytokine assay, histology and other analyses demonstrated the integrated anti-cancer mechanisms. The developed DualADC could provide a promising targeted chemo-immunotherapy for TNBC patients in the future.

TNBCs are characterized by the absence of estrogen receptor (ER), progesterone receptor (PR), and human epidermal growth factor receptor 2 (HER2) expression. To develop effective targeted therapies, numerous surface receptors have been explored. Recently, the transmembrane protein CD276 (B7-H3, Uniprot: Q5ZPR3), comprised of two Ig-like V-type and two Ig-like C2-type extracellular domains, was detected in over 80% of breast cancer tissues. This example revealed high expression of CD276 in over 60% of TNBC patients (126 cases) and various cell lines representing different subtypes while minimal to low expression levels in 33 normal human organs examined. Further, CD276 has been implicated to associate with angiogenesis, invasion, metastasis and poor prognosis in cancer patients. Furthermore, CD276 is an immune checkpoint molecule that inhibits the secretion of effector cytokines (IFN-γ, TNF-α, IL-4), as well as the immune function of natural killer (NK) and T cells. The combined anti-CD276 enoblituzumab/anti-PD-1 retifanlimab, vobramitamab duocarmazine, and bispecific antibody targeting CD276/CD3 were evaluated to treat head/neck cancer in phase I trial (NCT02475213) or multiple cancers in phase I/II trial (MGD009, MGC018). It is demonstrated herein that targeting CD276 could cover the majority of TNBC patients and upregulate tumoral immunity, rendering it a promising therapeutic strategy for aggressive TNBCs. Therefore, developed and engineered herein is a new CD276 mAb to construct a combined chemo-immunotherapy.

Toll-like receptor (TLR) 7/8 agonists play a pivotal role in recruiting and activating immune cells within the immunologically “cold” tumor microenvironment (TME). These agonists exhibit lower toxicity compared to immune checkpoint blockers (ICBs), such as anti-PD-1/PD-L1 mAbs. Furthermore, TLR 7/8 agonists have been found to impede cancer cell proliferation, induce apoptosis, and stimulate the release of cytokines (e.g., IL-2/6/8/10/12/18, IFN-α/γ, TNF-α) by immune cells; Table 1. Despite these promising immunotherapeutic effects, the administration of free TLR agonists lacking tumor selectivity may induce a potentially lethal cytokine storm and other adverse side effects. To overcome this challenge, used herein was anti-CD276 mAb to precisely deliver TLR 7/8 agonists, thereby fostering the upregulation of tumoral immunity in TNBC.

TABLE 1
Serum cytokine analysis of dual-payload ADC (chimeric CD276
mAb-MMAF/IMQ) treated immunocompetent models.

| | | Single | Single | Dual | Dual | Dual |
|---|---|---|---|---|---|---|
| | | ADC | ADC | ADC | ADC | ADC |
| Serum | | (mAb-MMAF) | (mAb-IMQ) | (mAb-MMAF/IMQ) | (mAb-MMAF/IMQ) | (mAb-MMAF/IMQ) |
| Cytokines | Saline | 8 mg/kg | 8 mg/kg | 8 mg/kg | 16 mg/kg | 24 mg/kg |
| IFN- | 65.61 | 80.43 | 93.21 | 62.92 | 81.66 | 64.26 |
| gamma | 66.29 | 88.62 | 98.67 | 66.52 | 86.86 | 61.58 |
| | 65.16 | 63.81 | 62.03 | 62.47 | 85.11 | 67.44 |
| | 62.25 | 71.60 | 61.58 | 58.51 | 82.64 | 64.71 |
| IL-6 | 485.60 | 608.48 | 789.46 | 390.37 | 647.00 | 454.04 |
| | 465.76 | 729.87 | 893.73 | 423.50 | 686.65 | 416.03 |
| | 419.76 | 419.76 | 406.77 | 416.03 | 644.70 | 431.04 |
| | 408.62 | 485.60 | 431.04 | 377.83 | 677.22 | 434.84 |
| TNF-alpha | 85.39 | 121.37 | 261.58 | 78.60 | 169.95 | 147.03 |
| | 112.62 | 188.66 | 245.76 | 118.26 | 159.78 | 105.81 |
| | 114.26 | <178.125 | 103.14 | 108.41 | 160.94 | 118.26 |
| | 84.30 | 62.85 | 101.32 | 89.64 | 138.55 | 137.20 |
| IL-10 | 190.36 | 392.39 | 502.20 | 174.36 | 461.96 | 179.78 |
| | 195.53 | 151.65 | 461.96 | 215.48 | 482.32 | 133.28 |
| | 205.64 | 120.21 | 113.36 | 190.36 | 449.18 | 157.50 |
| | 185.12 | 179.78 | 113.36 | 91.25 | 442.72 | 157.50 |
| IL-2 | <102.34 | <102.34 | 138.50 | <102.34 | 132.61 | 178.88 |
| | <102.34 | <102.34 | 125.45 | <102.34 | 131.57 | 142.11 |
| | <102.34 | <102.34 | <102.34 | <102.34 | 141.23 | <102.34 |
| | <102.34 | <102.34 | <102.34 | <102.34 | 136.60 | <102.34 |
| CCL4 | 69.44 | 58.93 | 84.59 | 69.77 | 744.48 | 1239.30 |
| | 68.04 | 55.09 | 82.60 | 89.08 | 954.64 | 406.87 |
| | 57.94 | 50.79 | 157.63 | 83.79 | 384.31 | 114.91 |
| | 55.82 | 56.60 | 59.12 | 80.79 | 368.29 | 7083.42 |
| IL-12p70 | 96.40 | 92.34 | 173.60 | 92.34 | 122.36 | 366.96 |
| | 100.28 | 88.08 | 168.24 | 132.64 | 126.88 | 253.54 |
| | 91.30 | 47.58 | 94.39 | 80.03 | 166.60 | 104.01 |
| | 83.59 | 90.24 | 92.34 | 82.42 | 168.24 | 104.01 |
| IL-18 | <2275 | <2275 | 721.10 | <2275 | 1011.11 | 4649.33 |
| | <2275 | <2275 | 1123.73 | 421.08 | 1149.82 | 3109.50 |
| | <2275 | <2275 | <2275 | <2275 | 2305.43 | <2275 |
| | <2275 | <2275 | <2275 | <2275 | 1965.86 | <2275 |
| CCL2 | 523.23 | 554.10 | 772.62 | 435.29 | 668.95 | 668.95 |
| | 512.02 | 465.55 | 782.14 | 533.95 | 590.28 | 566.71 |
| | 554.10 | <439.06 | 429.78 | 440.65 | 572.80 | 526.86 |
| | 440.65 | 429.78 | 440.65 | 346.49 | 512.02 | 554.10 |
| CCL11 | 1704.74 | 1000.69 | 2205.50 | 767.59 | 1543.53 | 2586.16 |
| | 1699.52 | 162.78 | 2325.47 | 1190.94 | 1647.08 | 1942.29 |
| | 1637.13 | 161.16 | 954.71 | 1028.61 | 2473.30 | 383.33 |
| | 1432.05 | 1909.72 | 914.63 | 861.97 | 2414.77 | 1704.74 |
| CXCL2 | 41.74 | 34.04 | 70.16 | 24.98 | 47.20 | 40.70 |
| | 45.26 | 26.39 | 73.10 | 44.52 | 48.86 | 30.37 |
| | 32.84 | <42.97 | 27.76 | 34.04 | 60.00 | 33.44 |
| | 31.62 | 29.08 | 14.41 | 33.44 | 54.50 | 38.01 |
| IL-17A | 209.30 | 392.20 | 289.04 | 65.51 | 292.63 | 116.18 |
| | 194.61 | 237.08 | 284.21 | 90.53 | 318.27 | 95.07 |
| | 153.06 | 76.07 | 99.49 | 138.97 | 342.81 | 90.53 |
| | 131.62 | 135.33 | 85.87 | 53.89 | 327.31 | 85.87 |
| CCL7 | 324.36 | 202.69 | 358.76 | 165.15 | 368.83 | 379.82 |
| | 360.10 | 48.77 | 407.58 | 280.30 | 364.61 | 254.33 |
| | 274.82 | 15.15 | 270.26 | 153.28 | 278.40 | 22.80 |
| | 230.98 | 271.10 | 234.06 | 9.51 | 235.40 | 361.93 |
| CCL3 | 28.72 | 65.28 | 106.22 | 27.03 | 59.34 | 90.73 |
| | 28.82 | 17.34 | 103.24 | 45.48 | 61.36 | 71.37 |
| | 23.76 | <29.69 | 40.50 | 37.42 | 93.27 | 35.16 |
| | 19.32 | 34.82 | 31.64 | 12.62 | 82.70 | 70.18 |
| CCL5 | 373.48 | 1252.18 | 1591.26 | 358.48 | 589.20 | 1395.40 |
| | 348.77 | 86.66 | 1600.16 | 837.35 | 649.42 | 750.44 |
| | 302.90 | 54.33 | 355.83 | 281.74 | 2066.33 | 242.98 |
| | 211.30 | 406.15 | 362.89 | 264.12 | 1842.52 | 1085.06 |
| CXCL1 | 126.18 | 94.91 | 159.44 | 76.16 | 110.14 | 794.23 |
| | 120.39 | 78.64 | 159.44 | 91.13 | 112.69 | 499.66 |
| | 91.76 | 61.43 | 82.37 | 84.87 | 189.94 | 105.04 |
| | 79.26 | 76.16 | 83.62 | 65.70 | 185.27 | 135.90 |
| GM-CSF | 162.19 | 166.77 | 234.29 | 175.33 | 186.94 | 383.57 |
| | 157.36 | 179.35 | 257.97 | 194.02 | 183.21 | 291.42 |
| | 171.15 | 127.97 | 141.03 | 146.83 | 221.37 | 186.94 |
| | 157.36 | 162.19 | 152.26 | 141.03 | 206.97 | 175.33 |
| IL-27 | 198.11 | 542.42 | 734.67 | 207.69 | 188.83 | 420.52 |
| | 180.74 | 402.11 | 703.20 | 256.13 | 179.08 | 331.32 |
| | 234.13 | 67.77 | 247.81 | 306.32 | 447.10 | 223.66 |
| | 169.65 | 453.51 | 226.98 | <130.47 | 409.94 | 278.58 |
| IL-23 | 455.76 | 6785.35 | 932.59 | 426.99 | 731.97 | 565.02 |
| | 459.34 | 1956.96 | 993.78 | 484.28 | 725.27 | 505.52 |
| | 426.99 | 416.13 | 419.75 | 568.49 | 1051.26 | 498.45 |
| | 419.75 | 484.28 | 434.21 | 470.05 | 1076.66 | 484.28 |
| IL-1 beta | 53.22 | 137.37 | 110.31 | 62.73 | 68.99 | 93.87 |
| | 54.50 | 79.20 | 105.23 | 74.73 | 66.97 | 74.73 |
| | 61.62 | 46.29 | 56.97 | 68.99 | 65.93 | 63.81 |
| | 55.75 | 53.86 | 56.97 | 53.22 | 64.88 | 61.62 |
| CXCL10 | > 2500 | 150.29 | > 2500 | > 2500 | > 2500 | > 2500 |
| | > 2500 | 77.20 | > 2500 | > 2500 | > 2500 | > 2500 |
| | 2500 | 132.59 | > 2500 | > 2500 | > 2500 | 130.50 |
| | >2500 | >2500 | 320.13 | > 2500 | > 2500 | > 2500 |
| IL-5 | 65.42 | 43.45 | 92.70 | <160.94 | 38.63 | 83.82 |
| | 72.61 | <160.94 | 76.15 | 30.29 | 45.03 | 37.00 |
| | 23.25 | <160.94 | 15.71 | 58.05 | 339.12 | 17.66 |
| | 9.51 | 40.25 | 27.70 | 42.66 | 271.40 | 13.72 |
| IL-22 | 593.39 | 1594.55 | 1289.96 | 670.36 | 1196.16 | 718.15 |
| | 593.39 | 958.74 | 1184.30 | 758.46 | 1243.29 | 621.66 |
| | 607.57 | 677.24 | 550.29 | 724.91 | 1687.23 | 690.95 |
| | 607.57 | 965.00 | 521.07 | 521.07 | 1725.00 | 745.09 |
| IL-9 | <1376.56 | 2652.65 | 2797.12 | <1376.56 | <1376.56 | 2088.79 |
| | <1376.56 | <1376.56 | 2747.31 | <1376.56 | <1376.56 | <1376.56 |
| | <1376.56 | <1376.56 | <1376.56 | <1376.56 | 2394.72 | <1376.56 |
| | <1376.56 | <1376.56 | <1376.56 | <1376.56 | 2202.83 | <1376.56 |
| IL-13 | 182.89 | 158.98 | 1193.13 | 157.63 | 178.54 | 284.77 |
| | 178.54 | 212.63 | 1119.14 | 185.09 | 186.56 | 204.76 |
| | 178.54 | 145.12 | 197.83 | 192.15 | 463.30 | 174.24 |
| | 150.98 | 248.32 | 186.56 | 147.71 | 351.76 | 209.46 |

The 4T1-FLuc xenografted female BALB/cJ mice were treated with dual-payload ADC (8, 16, 24 mg/kg mAb-MMAF/IMQ), mAb-MMAF (control), mAb-IMQ (control), and saline (control) via i.v. injection through the tail vein. n = 6-8.
Luminex assay using cytokine & chemokine multiplexing procartaplex panel was used to titrate the cytokines in serum post treatment. n = 4.

The clinical effectiveness of single-payload ADCs can be compromised by the unpredictable compensatory mechanisms, emergence of drug resistance during prolonged treatment, and inherent heterogeneity of cancers. To overcome these limits of traditional ADCs and address the off-target-induced immune toxicity of free TLR agonists, thereby improving tumor treatment efficacy, an advanced conjugation platform of dual-payload ADC (named DualADC) was established in which one mAb carries both highly cytotoxic chemotherapy and immunotherapy.

The objective herein was to develop and evaluate an innovative immune checkpoint CD276-targeted dual-payload ADC for chemo-immunotherapy of TNBCs. DualADC integrating cancer proliferation inhibition, tumoral cytokine enhancement, immune cell reactivation and TME modulation can effectively eradicate TNBC cells in vivo. A unique mAb with cross activity was developed and further engineered to target CD276⁺ TNBC patients. The platform was established to conjugate the engineered mAb with synergistic dual therapies through two linkers. This all-in-one ADC reduced tumor burden by 90-100% in TNBC xenografted mouse models including patient-derived xenograft (PDX) models. The DualADC developed herein represents a viable strategy for the treatment of aggressive TNBCs.

### Materials and Methods

### Cell Lines and Culture Media

The human TNBC cell lines, including MDA-MB-231 (ATCC, Cat #HTB-26, RRID:CVCL_0062, Manassas, VA, USA), MDA-MB-468 (ATCC, Cat #HTB-132, RRID:CVCL_0419), MDA-MB-231-FLuc (GenTarget, Cat #SC059-Puro, RRID:CVCL_YZ80, San Diego, CA, USA), and MDA-MB-468-FLuc (GeneCopoeia, Cat #SL027, RRID:CVCL_C8XW, Rockville, MD, USA) were maintained in DMEM medium supplemented with 10% fetal bovine serum (FBS, v/v) and 1% Pen/Strep (v/v). The normal breast epithelium 184B5 cell line (ATCC, Cat #CRL-8799, RRID:CVCL_4688) was maintained in MEGM bullet kit growth medium (Lonza, Walkersville, MD, USA) supplemented with 5% FBS. The mouse TNBC cell lines 4T1 (ATCC, Cat #CRL-2539, RRID:CVCL_0125) and 4T1-FLuc (ATCC, Cat #CRL-2539-LUC2, RRID:CVCL_5185) were cultivated in RPMI-1640 medium supplemented with 10% FBS and 1% P/S. The Mouse carrying TNBC PDX (Jackson Lab, Cat #J000103917) were purchased from Jackson Lab (Bar Harbor, ME, USA), then PDX was harvested, passaged and maintained in NSG mice or freshly frozen and stored in a liquid nitrogen tank. The Expi293 cells for chimeric CD276 mAb production were maintained in Expi293 Expression medium supplemented with 4 mM glutamax and 6 g/L glucose. The CHO cells producing humanized anti-CD276 mAb were kept in Dynamis medium supplemented with 4 mM L-glutamine and 6 g/L glucose. All cell lines were incubated at 37° C. and 5% or 8% CO₂ in a humidified incubator (Eppendorf, Enfield, CT, USA). All media, supplements and bioreagents used in this study were purchased from Fisher Scientific (Waltham, MA, USA) unless otherwise specified. All the cell lines or PDX lines were purchased commercially, authenticated with genetics profiling for polymorphic short tandem repeat analysis at University Genomics Core, and confirmed with in-house mycoplasma test using PCR primers that amplify sequences of 16S rRNA genes. The latest test date of all cell banks with stock vials of 30-100 was Nov. 21, 2022. The length of time between cell thaw of the tested cell bank and use in our experiment was 2-3 weeks.

### Anti-CD276 mAb Development, Engineering and Production

The peptide cloned from the extracellular domain (Leu29-Pro245) of human CD276 was used to stimulate immune response in BALB/cJ mice (Jackson Lab). Blood samples were collected from the tail vein to titrate serum concentration of CD276 mAb using ELISA at 14-21 days post-immunization. Once mAb was detected, splenocytes were harvested and fused with myeloma cells Sp2/0-Ag14 (ATCC) to generate hybridoma, followed by limiting dilution with a seeding density of 1~4 cells/well in 96-well plates. The top hybridoma clones with high mAb titer and CD276 binding were sequenced. To minimize the immunogenicity, improve serum stability and enhance Fc-mediated antibody effector functions, the murine anti-human CD276 mAb was first engineered by constructing a chimeric CD276 mAb which grafts the complementary-determining region (CDR) with a truncated Fc region of human IgG1. Then a humanized CD276 mAb was constructed by combining the murine framework regions (FRs) and three human CDRs.

The transient production system was used to generate chimeric CD276 mAb from Expi293F cells in a 2-L stirred-tank bioreactor (Temp of 37° C., Agt of 140 rpm, DO of 40%, and pH of 7.2) or shaker flask culture (Temp of 37° C., Agt of 130 rpm, CO₂ of 8%). The stable CHO production cells were developed to produce humanized CD276 mAb under similar conditions as above. A liquid chromatography system (Bio-Rad, Hercules, CA, USA), which is equipped with Bio-Scale Mini UNOsphere SUPrA affinity chromatography cartridges (Protein A column, Bio-Rad), was utilized for mAb purification following our established procedure. The mobile phase A of 0.02 M sodium phosphate and 0.02 M sodium citrate (pH 7.5) and phase B (elution buffer) of 0.1 M sodium chloride and 0.02 M sodium citrate (pH 3.0) were used.

### Conjugation of CD276-Targeting Single-Payload and Dual-Payload ADCs

Single-Payload ADC (CD276 mAb-MMAF)

A bifunctional dibromomaleimide (DBM) linker was used to cross-link mAb interchain cysteines and carry four MMAF (Monomethylauristatin F) drugs. The 5 mM TCEP, dissolved in DI water at pH 7, and 5 mg/mL mAb in PBS were mixed with a molar ratio of 44:1 and performed at 37° C. for 0.5 hrs to completely reduce the disulfide bonds in mAb. The 10 mM commercial DBM-MMAF payload was prepared in DMSO, and 7 molar equivalents were added to the completely reduced mAb with 1-hr incubation at room temperature. The crude ADC was purified using a Protein A column with a liquid chromatography system. The LC-purified ADC was buffer-exchanged into PBS using a 2 kDa Slide-A-Lyzer Dialysis Cassette and concentrated to a higher concentration with 10 kDa MWCO PES concentrators. The ADC purity, drug-antibody ratio (DAR) and homogeneity were tested using HPLC (Shimadzu, Columbia, MD, USA) equipped with a MAbPac hydrophobicity interaction chromatography (HIC)-butyl column (5 μm, 4.6×100 mm). The mobile phase A of 2 M ammonium sulfate and 100 mM sodium phosphate at pH 7.0 and mobile phase B of 100 mM sodium phosphate at pH 7.0 were used in HPLC analysis. The UV/Vis spectroscopy was used to calculate DAR as an alternative approach. The purified ADC was filtered through a 0.2 μm PES syringe filter (basix) before mice intravenous injection and stored at 4° C. for short-term storage.

Single-Payload ADC (CD276 mAb-IMQ)

In this conjugation platform, NHS-azide and NHS-phosphine reagents (Thermo Scientific) were used to conjugate mAb and IMQ. Mixture A is created by combining 5 mg/mL of mAb in PBS with 10 mM of NHS-Phosphine linker at a molar ratio of 1:14. In mixture B, 10 mM of NHS-azide linker and 10 mM of IMQ were combined in 500 μL PBS at a molar ratio of 14:22.4. Mixture A and Mixture B were performed at 37° C. for 2 hrs, respectively. A 10 kDa MWCO PES concentrator was used to purify phosphine-labeled mAb in Mixture A from excess NHS-phosphine. Mix the phosphine-labeled mAb with 500 μL of Mixture B, and incubate at 37° C. for 2 hrs to synthesize mAb-IMQ ADC. The purification and concentration steps are identical to those used for the mAb-MMAF ADC.

Dual-Payload ADC (CD276 mAb-MMAF/IMQ)

The 5 mg/mL of synthesized mAb-MMAF in PBS was mixed with 10 mM of NHS-Phosphine linker at a molar ratio of 1:14, forming mixture A. Mixture B was created by combining 10 mM of NHS-azide linker and 10 mM of IMQ in 500 μL PBS at a molar ratio of 14:22.4. Mixture A and Mixture B were incubated at 37° C. for 2 hrs each. Mixture A was then applied to 10 kDa MWCO PES concentrators to remove the free linker. Mixture B was added to the phosphine-labeled mAb-MMAF ADC and incubated at 37° C. for 2 hrs. The crude DualPayload ADC was subsequently purified, buffer exchanged to PBS, and concentrated as described above.

### Flow Cytometry Analysis of Cell Surface Binding

The surface binding of this anti-CD276 mAb in TNBC cell lines (MDA-MB-231, MDA-MB-468, 4T1) was tested by flow cytometry analysis following reported protocols. The CD276 mAb was labeled with a fluorescent Alexa Fluor™ 647 labeling kit (Life Technologies, part of Fisher). One million human or mouse TNBC cells were stained with 5 μg of anti-CD276 mAb-AF647 at 37° C. for 60 mins. After washing three times with PBS, the stained cells were analyzed using a BD LSRII flow cytometer (BD Biosciences, San Jose, CA, USA), with FlowJo software for data processing. In the analysis of the surface binding rate of our anti-CD276 mAb, gating was set where TNBC cells without mAb staining have <0.5% fluorescent population. In the analysis of TNBC specificity by anti-CD276 mAb, the standard forward and side scatter gating was applied with anti-HER2 mAb as negative control.

### Live-Cell Confocal Microscopy

The surface binding and internalization of CD276 mAbs were evaluated using live-cell confocal imaging, following our established protocols. TNBC MDA-MB-468 cells were cultured in 35 mm glass bottom dishes (Cellvis, Mountain View, CA, USA) at a density of 1×10⁴ cells per dish in 1.5 mL of medium. To visualize the cytoplasm and nucleus, BacMam GFP Transduction Control (Invitrogen, CA, USA) and NucBlue™ Live ReadyProbes™ Reagent (Invitrogen, CA, USA) were used for staining following manufactures protocols, respectively. Next, the Cy5.5-labeled CD276 mAb was added to the cells at a final concentration of 1 μg per mL. Live-cell images were captured at 2-12 hrs after the addition of mAb using a Nikon A1R-HD25 confocal microscope (Nikon USA, Melville, NY, USA).

### In Vitro Cytotoxicity Assay

The human and mouse TNBC cells were seeded in 96-well plates with a density of 10,000 cells/well for MDA-MB-468, 1,000 cells/well for MDA-MB-231 and 500 cells/well for 4T1. The free MMAF, mAb-MMAF and mAb-MMAF/IMQ were added into each well to reach the final dosages of 0-300 or 400 nM. Higher concentrations, i.e. 0-20 μM, of free IMQ and mAb-IMQ, were tested. The treated cells were incubated at 37° C. and 5% CO₂ for five days and the cell viability was analyzed using MTT Cell Proliferation Assay kit.

### Patient-Derived Xenograft (PDX) Model and In Vivo Treatment

The CD276⁺⁺⁺ TNBC PDX, identified from the Jackson Lab PDX lines through transcript analysis and IHC staining, was used following our published procedure. Briefly, the PDX tumors were minced into small fragments (1×1×1 mm³), loaded into a 1-mL sterile syringe connected with a 13G needle (BD, Franklin Lakes, NJ, USA), and s.c. injected into the rear flank of 5~7-week-old NSG female mice with 40-50-μL implantation for each mouse. The anti-TNBC efficacy of humanized CD276 mAb-derived single-payload and dual-payload ADCs was evaluated in the PDX model when tumor volume reached 25-50 mm³ by i.v. injecting saline, 16 mg/kg of mAb-MMAF, or 16 mg/kg mAb-MMAF/IMQ on a Q7Dx3 schedule (n=5-7) until tumor volume reached >1,500 mm³.

### Cell Line-Derived Xenografts and In Vivo Treatment

### 4T1-FLuc Xenografted Immunocompetent Models

The 4T1-FLuc cells (2×10⁶ cells per mouse) were subcutaneously (s.c.) injected into 6-week BALB/cJ female mice. When the average tumor volume reached 20-50 mm³, mice were randomized into multiple groups (n=5, or 7 or 8) and i.v. injected with saline (control), 8 mg/kg of mAb, mAb-IMQ, mAb-MMAF (controls), or 8, 16 or 24 mg/kg of mAb-MMAF/IMQ following schedules of Q7Dx4. Tumor size was measured by an electric caliper or IVIS imaging and tumor volume was calculated as (length×width2)/2. Tumor and mice body weight were monitored twice a week. Mice were sacrificed when tumor volume in the control group reached >1,000 mm³ or ulceration >2 mm. All tumors, main organs (brain, heart, lung, kidneys, liver, and spleen), whole blood, or serum were collected for further analysis.

### MDA-MB-231-FLuc Xenografted Immunocompromised Models

The 5×10⁶ of MDA-MB-231-FLuc cells were s.c. injected into 6-week-old NSG (NOD.Cg-Prkdc^{scid} Il2rg^{tm1Wjl}/SzJ) female mice. When tumor volume reached 50-100 mm³, mice were randomized (n=5) and i.v. administrated with saline (control), 16 mg/kg mAb-MMAF, or 16 mg/kg mAb-MMAF/IMQ following the schedule of Q5Dx5. Tumor volume was monitored with electric caliper or IVIS imaging and mice body weight was measured twice a week. Mice were sacrificed to collect tumors, organs, blood or serum for post treatment analysis when tumor volume reached >1,000 mm³ or ulceration was >2 mm.

### In Vivo Imaging System (IVIS)

When the tumor volume reached 50-100 mm³, 40 μg of engineered anti-CD276 mAbs, which were labeled with cyanine-5.5 fluorescent dye (Lumiprobe, Hunt Valley, MD, USA), were i.v. injected into the mouse via tail vein. The live-animal images were captured at 24 hrs post-injection with i.p. injection of FLuc substrate. Then the mice were sacrificed to harvest major organs, including brain, heart, lung, spleen, liver, and kidneys, and tumors for ex vivo imaging. The excitation/emission wavelength of 660/710 nm and exposure time of 5 seconds were used in IVIS imaging.

### Luminex Assay

To analyze the tumoral or serum cytokines, a Luminex-based multiplexing assay (Luminex Corporate, Austin, TX, USA) was employed. The pre-configured (EPX070-20835-901, EPX260-26088-901) and customized (PPX-03, PPX-13) chemocytokines assay kits purchased from Thermo Fisher (Waltham, MA) covering 3, 7, 13, or 26 plex and all assay reagents were prepared following manufacturer's procedure. The Luminex assays of tissue or serum samples (n=3) were performed in 96-well plates provided with the kits, and the raw data of mean fluorescence intensity (MFI) were read using the Luminex MAGPIX under the XPONENT software.

### Immunohistochemistry (IHC) Staining

The TNBC (ER⁻/PR⁻/HER2⁻) patient tissue microarray (TMA, Cat #BR1303, 126 cores) and 33 human normal organs tissue microarray (Cat #FDA662c) was purchased from US Biomax (Derwood, MD, USA) to detect CD276 expression or the potential non-specific binding of our humanized CD276 mAb with IHC staining (26,27). The harvested tumor tissues were either fresh frozen or fixed in 4% formalin. The fixed tissues were dehydrated through graded ethanol solutions, embedded in paraffin blocks, and sectioned with 4-5 μm thickness using a microtome and mounted onto glass slides.

During IHC staining, the TMA slide or tumor parafilm sectioned slide was first baked overnight at 60° C., de-paraffinized with xylene, and hydrated in ethanol and deionized water. Then the sectioned tissues were subjected to antigen retrieval in 0.01 M sodium citrate buffer (pH 6.0) for 5 mins, washed gently in deionized water, and transferred to a solution comprised of 0.05 M Tris, 0.15 M NaCl and 0.1% Triton-X-100 (TBST, pH 7.6). The endogenous peroxidase was blocked with 3% H₂O₂ for 15 mins and slides were incubated with 5% normal goat serum for 45 mins to reduce nonspecific background staining. All slides were incubated at 4° C. overnight with anti-CD276 antibody (Abcam, Rabbit monoclonal, Cat #ab226256, RRID:AB_3069232, 1/500 dilution) or our humanized CD276 mAb. After washing with TBST, the slide was incubated with goat anti-rabbit secondary antibody conjugated with HRP (Abcam, Cat #ab6721, RRID:AB_856214, 1:1000). Finally, the stained TMA slide was scanned with Lionheart FX Automated Microscope (BioTek, Winooski, VT, USA) and images were processed with Gen5 software. Following our previously established method (26), ImageJ was used to score the CD276 expression in the stained TMA. Briefly, the expression score of CD276 was calculated as (red_{intensity}−blue_{intensity})/blue_{intensity}×100 with the definition of high expression with score of >10, medium expression with score of 6-10, low expression with score of 3-6, and no or minimal expression with score of 0-3.

### HPLC Characterization of ADCs

The analysis of purity and the drug-antibody ratio (DAR) of the ADC was conducted using a High-Performance Liquid Chromatography (HPLC) system from Shimadzu (Columbia, MD, USA), utilizing a MAbPac HIC-Butyl column with dimensions of 5 μm, 4.6×100 mm. Two mobile phases were applied in the analysis: mobile phase A, containing 1.5 M ammonium sulfate and 50 mM sodium phosphate at a pH of 7.0, and mobile phase B, containing 50 mM sodium phosphate at a pH of 7.0. The procedure was carried out at a temperature of 25° C. and a consistent flow rate of 1.0 mL/min.

### MOLDI-TOF MS Characterization of ADCs

Matrix-Assisted Laser Desorption/Ionization Time-of-Flight (MALDI-TOF) mass spectrometry was applied to characterize and confirm the structure of our ADCs. Both ADC and mAb samples were diluted with HPLC-grade water to 2 mg/mL. 10 mg of sinapinic acid was dissolved in 1 mL HPLC-grade water containing 50% ACN (acetonitrile) and 0.1% TFA (trifluoroacetic acid). 10 μL of the samples were mixed with 10 μL of the sinapinic acid matrix solution, and then 4 μL of the mixture was spotted onto an MSP 96 target and air-dried. The samples were analyzed with a MALDI-TOF mass spectrometer (microflex LRF, BRUKER) in linear positive mode. The data were processed using flexAnalysis software.

### Western Blotting

The TNBC cells were washed three times with cold PBS and lysed using RIPA buffer to extract cellular proteins. The protein concentration was quantified using the bicinchoninic acid (BCA) method with a Pierce protein assay kit (Pierce, Cambridge, NJ). Next, 30 μg of proteins were loaded per lane onto a gradient SDS-PAGE, NuPAGE 4-12% gradient gel (Invitrogen, CA, USA), along with a protein size marker (Bio-Rad Precision Plus) to separate the proteins by molecular weight. The separated proteins were then transferred onto a PVDF membrane using a Bio-Rad power supply (Bio-Rad Laboratories, CA, USA) at a constant voltage of 100V for 90 minutes. After transfer, the PVDF membrane was blocked with 5% non-fat milk in TBST buffer and agitated at room temperature (RT) for 1 hr. For primary antibodies, we used CD276 (Dilution 1:1000, AB134161, Abcam, Cambridge, UK) and β-actin (1:2000, sc-47778, Santa Cruz, CA, USA). The membrane was then incubated overnight at 4° C. with continuous agitation. On the following day, the primary antibodies were discarded, and the membrane was washed three times with TBST buffer on a shaker, with each wash lasting 5, 5, and 10 minutes, respectively. After the washing steps, horseradish peroxidase (HRP)-conjugated secondary antibodies (Dilution 1:2000) specific to Mouse or Rabbit from Cell Signaling Technology (CST Inc, Danvers, MA, USA) in 3% non-fat milk was applied to the membrane accordingly for 1 hr at RT. After discarding the secondary antibody, the membrane was washed three times with TBST for 5, 5, and 10 minutes, respectively. Finally, the protein bands were visualized and quantified using an Odyssey Fc imaging system (LI-COR Biosciences, NE, USA). The expression of CD276 in TNBC cells was compared to the internal control, β-actin.

### Whole Blood Analysis

To evaluate the possible peripheral toxicity of our developed anti-CD276 mAb, free TLR 7/8 agonist, and ADCs, whole blood analysis was performed using BALB/cJ mice. Specifically, 8 mg/kg mAb, 8 mg/kg IMQ, and 8-24 mg/kg single-payload ADC or dual-payload ADC were i.v. injected into each mouse through the tail vein with saline as control. Ten or 21 days after injection, the blood samples were collected via cardiac puncture for whole blood analysis using HemaVet 950FS (Drew Scientific, Miami Lakes, FL, USA). The blood levels of leukocytes (white blood cell, neutrophil, lymphocyte, monocyte, eosinophils), erythrocytes (red blood cell, hemoglobin, mean corpuscular hemoglobin), and thrombocytes (platelet) were measured and analyzed.

### Hemotoxylin and Eosin (H&E) Staining

The slides of paraffin sectioned organs were dewaxed with xylene and hydrated with gradient ETOH (100%-50%) and dH₂O, followed by hematoxylin staining, dipping in 1% HCl and 70% ETOH, immersing in 1% NH₄OH for blue color development, and staining with eosin for 30 secs. Finally, the stained slides were dehydrated in 95% and 100% ethanol and cleared in xylene. The H&E-stained slides were imaged with Lionheart FX Automated Microscope (BioTek, Winooski, VT, USA).

### Single-Cell RNA Sequencing and Data Analysis

### Single-Cell Sample Preparation

The flash-frozen tissue samples (25 mg) were collected and stored at −80° C. until processing. Chromium Next GEM RNA Profiling Sample Fixation Kit (PN-1000414, 10× Genomics, Pleasanton, CA, USA) was used to fix tissue by mixing 1 mL of fixation buffer with 25 mg of tissue, followed by fine mince and incubation at 4° C. for 16-24 hours without agitation. After fixation, tissue samples were centrifuged, washed with chilled PBS, and resuspended in 1 mL of tissue resuspension buffer. The fixed tissues supplemented with pre-warmed dissociation buffer were dissociated using an Octo Dissociator according to the manufacturer's instructions. The dissociated tissue samples were filtered through a 30 μm filter, centrifuged and resuspended in 1 mL of chilled quenching buffer. Cell concentration was determined using an automated cell counter with fluorescent nucleic acid staining. The dissociated tumor samples were stored in 50% glycerol with enhancer at −80° C. until single-cell library generation.

### Single-Cell Library Construction

A 10× single-cell library was prepared using the Chromium Fixed RNA Kit (Cat #1000497, 10× Genomics). Briefly, ~16,500 cells were hybridized with probe hybridization to target the polyadenylated RNA, and loaded onto Chromium X (PN-1000326, 10× Genomics) to generate barcode-labeled single-cell gel beads-in-emulsion (GEMs) with a target cell count of ~10,000. GEMs were then lysis using a recovery agent to release the hybridized RNA. cDNA was synthesized, amplified, and followed by sample index PCR for indexing. The quality control of cDNA was evaluated using an Agilent Bioanalyzer aiming for the final library molecules with P5 and P7 priming sites used in Illumina sequencers. For each cDNA sample, single-cell library construction was started using the 10× barcoded ligated probe products, and sequenced using the NovaSeq 6000 flow cell 100-cycle kit (Illumina, San Diego, CA, USA) in a Read1:i7:i5:Read2 format of 28:10:10:90 bp at 10× Genomics. Demultiplexed fastq files were subsequently utilized for analysis.

### Data Preprocessing and Cell Annotation

Three FASTQ files stored raw reads information of index reads, forward reads from the paired-end sequencing, and reverse reads from paired-end sequencing were processed using 10× Genomics Cloud Analysis embedded in Cell Ranger Multi version 7.1.0 and the mouse reference genome mm10 2020-A were used for the alignment. The sequencing quality control (QC) was performed using Windows and Linux based FastQC (version 0.12.1). The count-by-gene matrix was analyzed using ‘Seurat’ (version 4.3.0.1, PMID: 34062119) package in R (version 4.3.0). The SCTransform function developed by Christoph Hafemeister and Rahul Satija was used to normalize and scale data. CellMarker 2.0, PanglaoDB and Tabula Muris database were used to annotate different cell types.

### Differential Expression Genes and GO Pathway Enrichment

Differential expression genes were identified between groups among all the cell types and the integrated cells using Seurat built-in function PrepSCTFindMarkers and FindMarkers. All the tests were operated based on Wilcoxon sum rank test. Notable, the parameters of min.pct and logfc.threshold were set to 0 for further GSEA pathway enrichment. The gseGO function embedded in clusterProfiler package (version 4.8.2) was used to operate GSEA pathway enrichment with the adjusted p value of 0.05, sourced from the Gene Ontology database (https://geneontology.org/).

### Pharmacokinetics (PK)

Five doses of CD276 mAb-MMAF/IMQ, including 4, 8, 16, 24 and 32 mg/kg, were i.v. injected to 4T1 xenografted BALB/cJ mice. About 10-15 μL of blood samples were collected from tail vein or submandibular or submental route at 0.5, 2, 8, 24, 48, 72, 120 hrs post-injection. The supernatants were used to titrate DualADC using ELISA with human CD276 receptor and anti-MMAF antibody (Fisher, Cat #PIMA542537, RRID:AB_2687990). The reported PK model was used to calculate the important parameter: clearance

$$( CL ) = \frac{DF}{2 aAUC} = \frac{{V}_{d}}{{k}_{e}} ,$$

volume of distribution

$$( {V}_{d} ) = \frac{CL ( {t}_{2} - {t}_{1} )}{{lnC}_{1} - {lnC}_{2}} ,$$

half life

$${t}_{1 / 2} = \frac{0.6 \; 93 {V}_{d}}{CL} ,$$

recommended dose

$$( D ) = {C}_{max \cdot desired} {k}_{e} {V}_{d} T \frac{1 - {e}^{- {k}_{e} τ}}{1 - {e}^{{k}_{e}} T} ,$$

and dosing interval

$$( τ ) = \frac{{lnC}_{max \cdot desired} - {lnC}_{min \cdot desired}}{{k}_{e}} + T .$$

### Results

### Overview of the Concept of Dual-Payload ADC

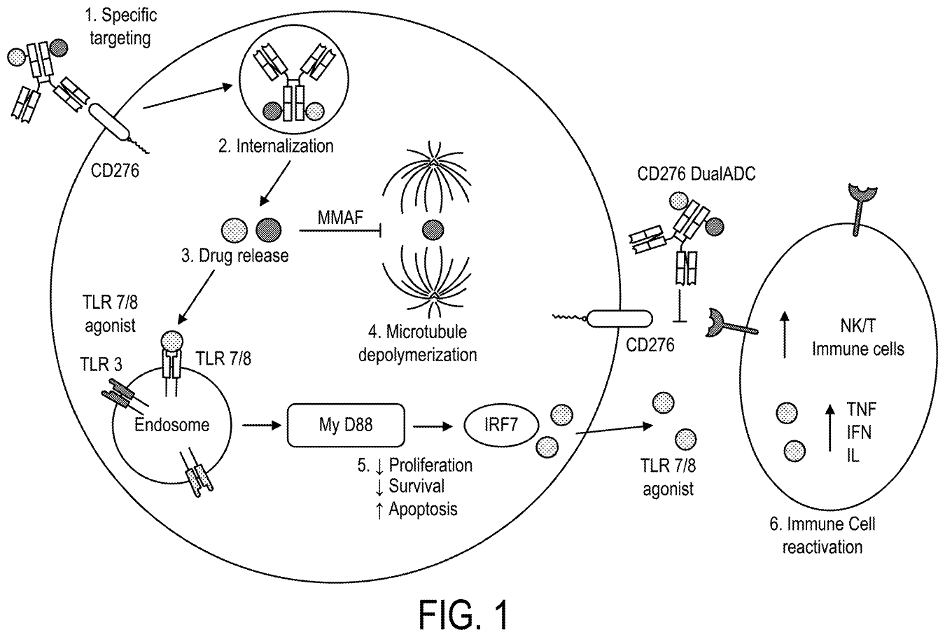

The concept of this CD276-targeted DualADC for chemo-immunotherapy was described in FIG. 1. The engineered CD276 mAb carrying potent molecule MMAF, or immune boosting reagent TLR 7/8 agonist IMQ, or both payloads, named as CD276 mAb-MMAF/IMQ in this study, targets the overexpressed surface receptor CD276 on TNBCs. After internalization via receptor-mediated endocytosis and lysosomal degradation, the payloads MMAF and IMQ are released into the cytoplasm. The intracellular MMAF leads to direct cancer cell death by blocking microtubulin polymerization, and IMQ inhibits cell proliferation via signaling pathway MyD88/IRF7 after targeting TLR7/8 in endosome as previously reported. The IMQ (free drug released from TNBC cell death or drug conjugated in ADCs) upregulates the cytokines' secretion by immune cells in TME. Additionally, the inhibition of CD276 immune checkpoint by mAb reactivates NK and T cells in the tumor.

### Development Dual-Payload ADC

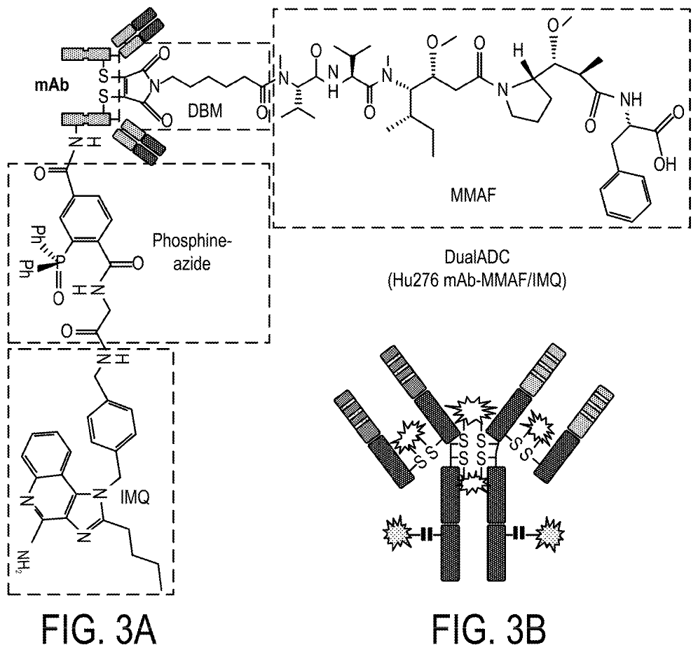

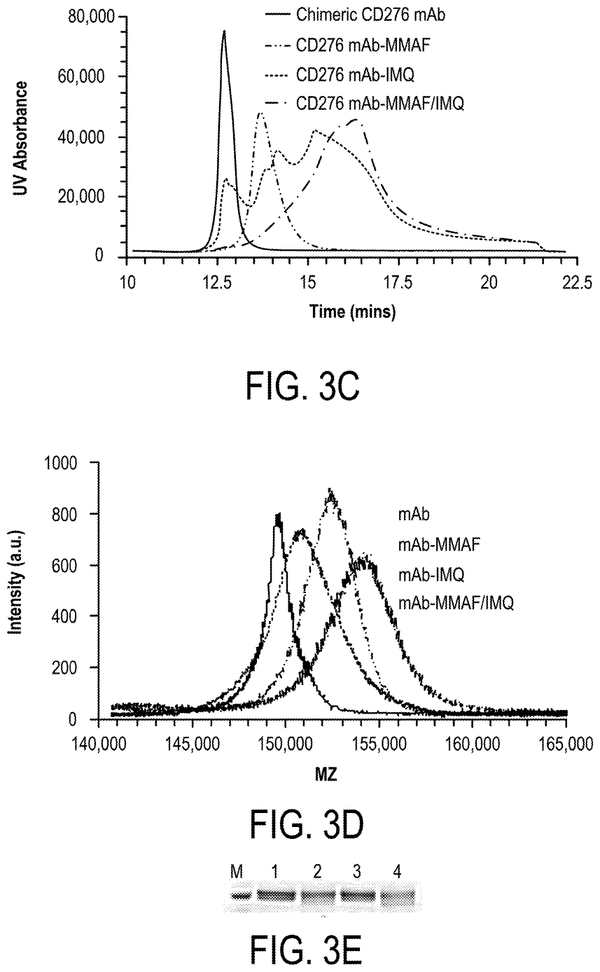

A new sequential conjugation procedure was developed to construct DualADC by linking mAb with MMAF using re-bridging dibromomaleimide (DBM) linker, followed with IMQ conjugation using phosphine-azide linker designed in this study (FIGS. 3A-B). HPLC characterizations showed the successful conjugations of both single payload (IMQ or MMAF) and double payloads (IMQ and MMAF) (FIG. 3C). MS evaluation confirmed the right structures of mAb, mAb-MMAF, mAb-IMQ, and mAb-MMAF/IMQ with the expected MZ values (FIG. 3D). The integrity of mAb and ADCs were confirmed in SDS-PAGE (FIG. 3E). The site-specific conjugation of MMAF had homogenous structure, purity of 95-100%, and DAR of 3-4 while random conjugation of IMQ showed a heterologous structure and DAR of 7-14. Random conjugation was used to optimize the condition and DAR (8-12) of IMQ to achieve high solubility and stability and tumoral immunity.

### In Vitro Anti-TNBC Cytotoxicity of ADCs

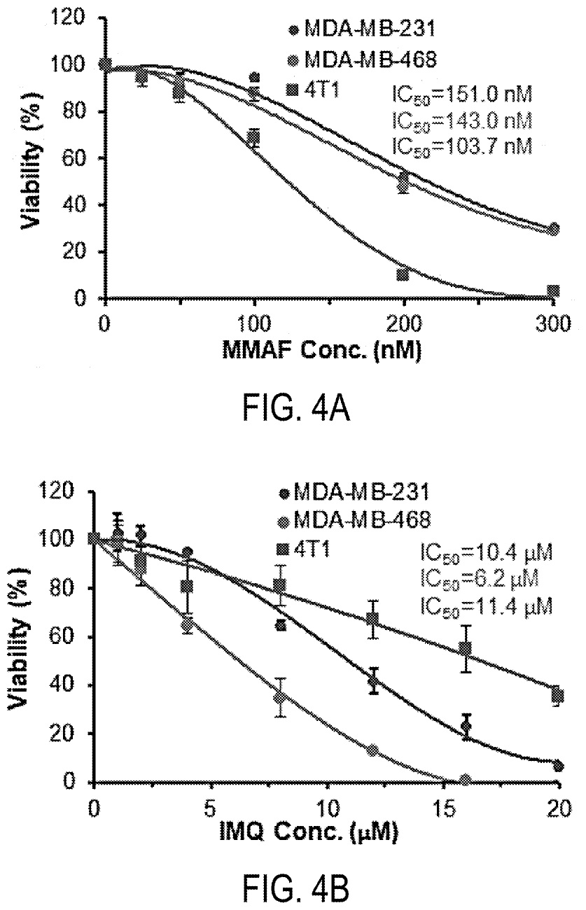

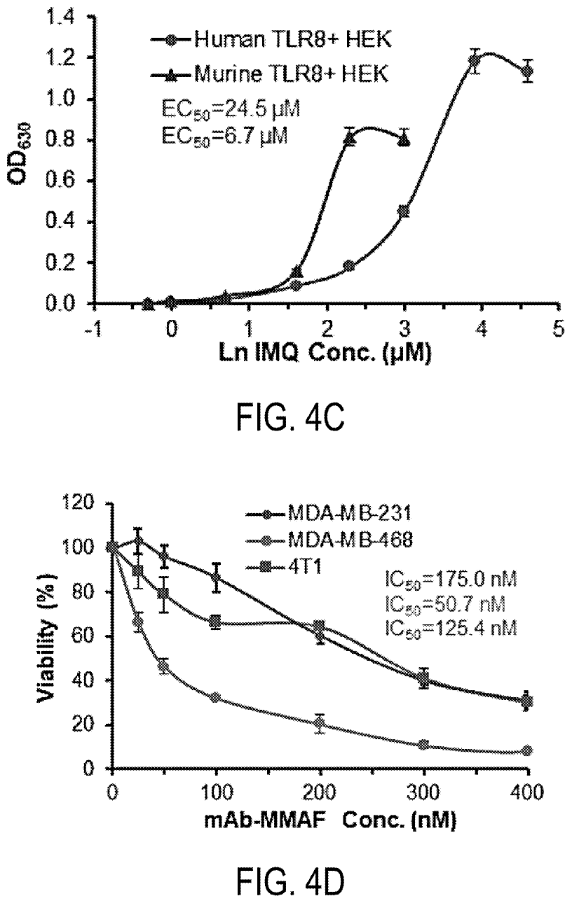

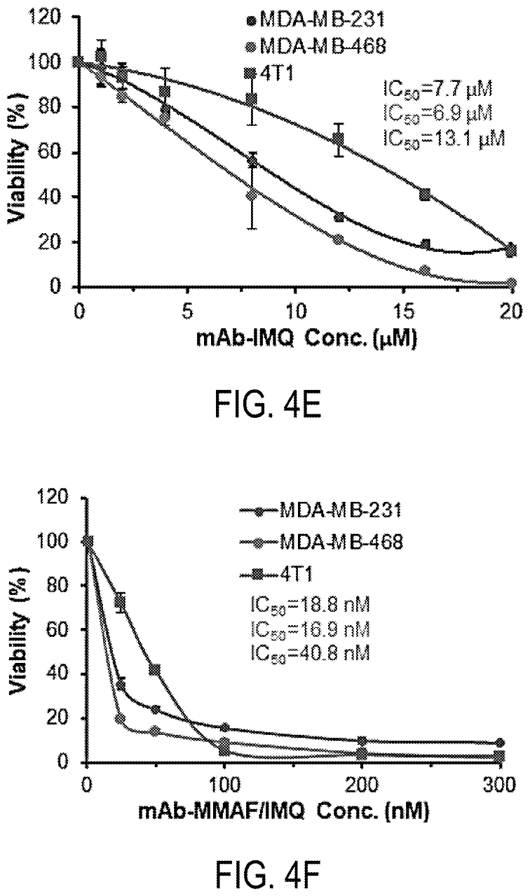

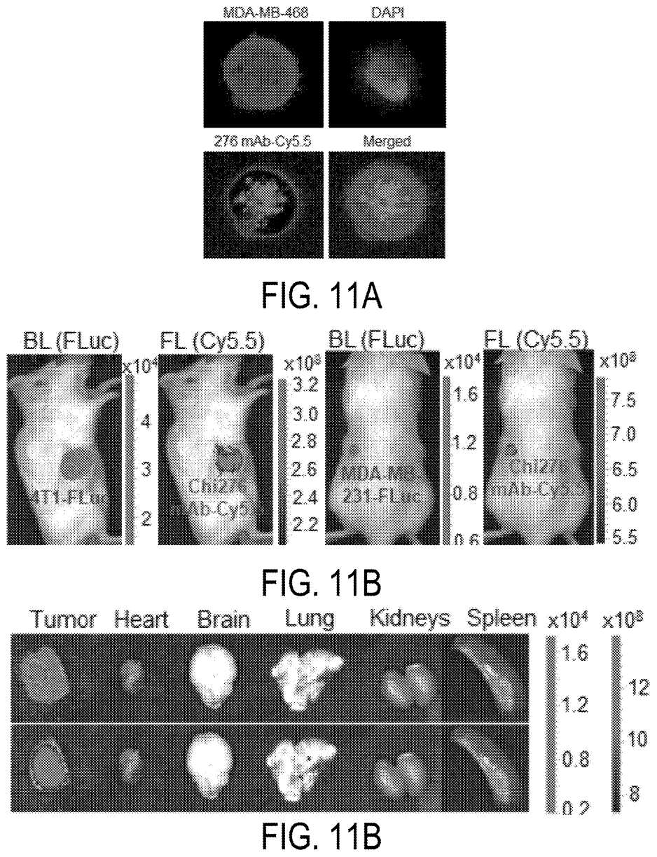

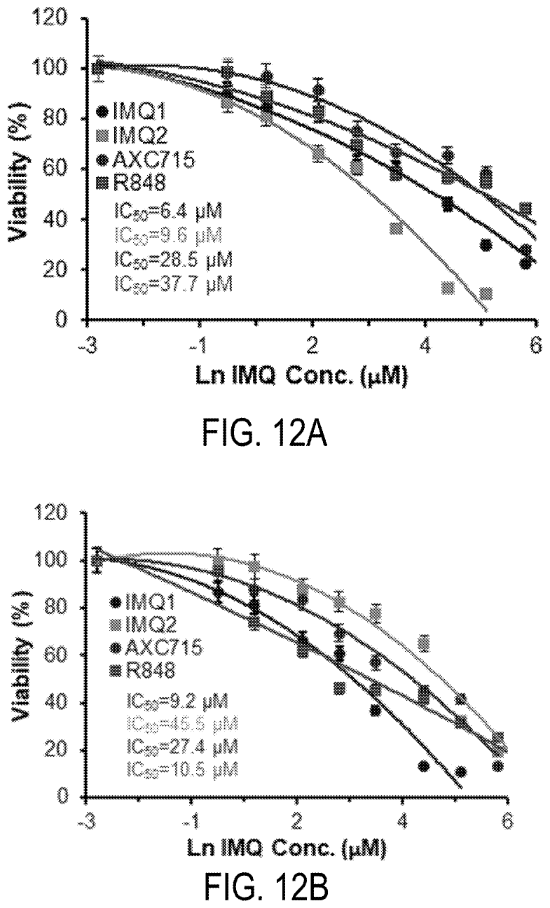

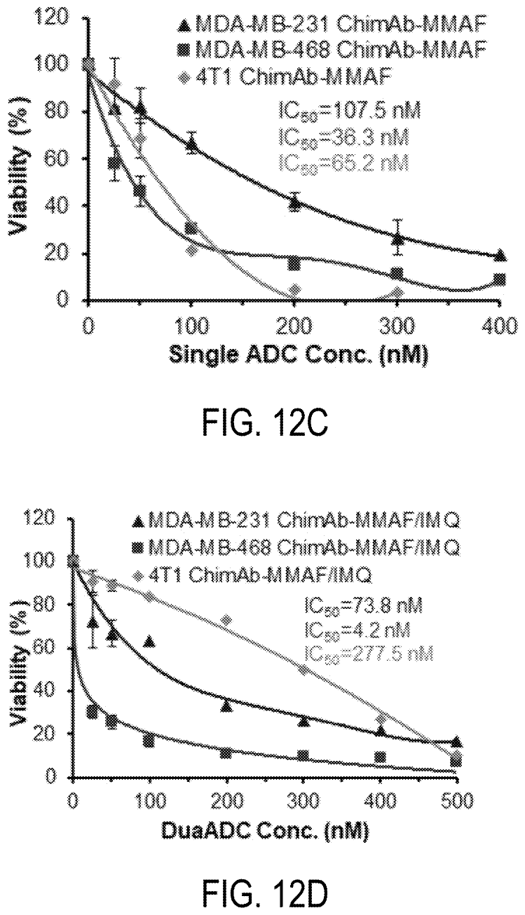

The cytotoxicity of free MMAF and TLR 7/8 agonists (control), CD276 mAb-MMAF and mAb-IMQ (controls), and dual-payload mAb-MMAF/IMQ was tested using MDA-MB-231, MDA-MB-468 and 4T1 cells. MMAF exhibited IC₅₀ values of 151.0, 143.0 and 103.7 nM for these three cell lines, respectively (FIG. 4A). IMQ with high TNBC cytotoxicity was identified from the tested TLR7/8 agonists, including two IMQs, AXC715 and R848 (FIG. 12). The IMQ1 showing IC₅₀ values of 10.4, 6.2 and 11.4 μM in three TNBC lines (FIG. 4B) was used in single- and dual-payload ADCs. IMQ had EC₅₀ values of 24.5 and 6.7 μM in TLR 8 overexpressing human and murine HEK 293 cells (FIG. 4C). The IC₅₀ values of humanized CD276 mAb (Hu276 mAb)-MMAF were 175.0 nM (MDA-MB-231), 50.7 nM (MDA-MB-468) and 125.4 nM (4T1) as described in FIG. 4D. Similar to free IMQ, Hu276 mAb-IMQ revealed IC₅₀ values of 7.7 μM (MDA-MB-231), 6.9 μM (MDA-MB-468), and 13.1 μM (4T1). The DualADC Hu276 mAb-MMAF/IMQ (FIG. 4F), with IC₅₀ values of 18.8 nM (MDA-MB-231), 16.9 nM (MDA-MB-468) and 40.8 nM (4T1), had the highest cytotoxicity or potency to TNBC cells as compared to free drugs and single-payload ADCs.

### In Vivo Anti-TNBC Efficacy of DualADC in TNBC PDX Models

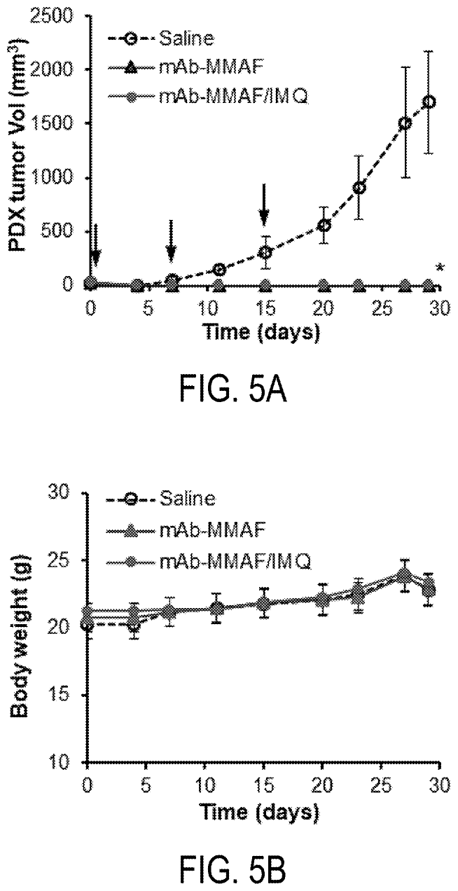

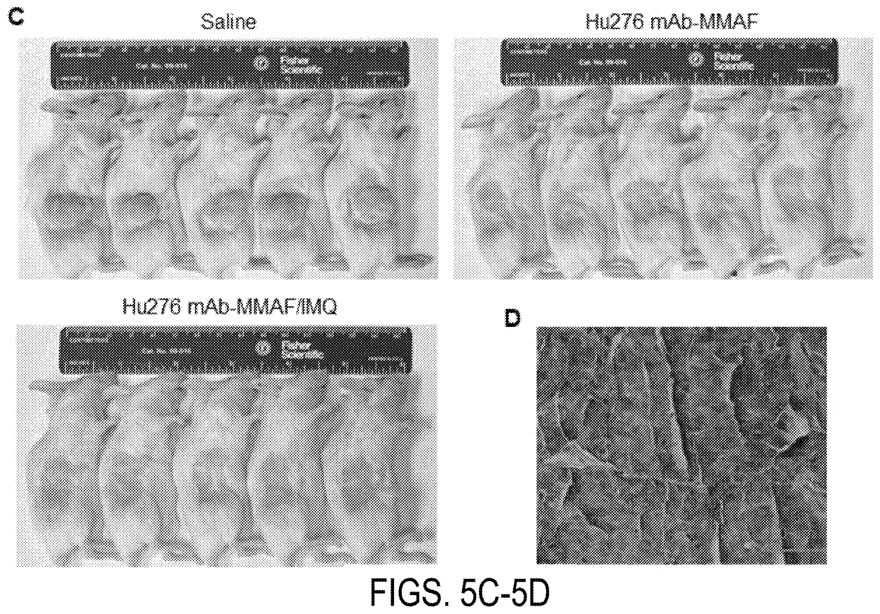

The PDX models capitulating tumor heterogeneity and tumor microenvironment are crucial to fully evaluate the newly developed targeted therapy. As shown in FIG. 5A, PDX tumor volume without treatment (saline group) reached ~1,700 mm³ on Day 29 post treatment. Both Hu276 mAb-MMAF and Hu276 mAb-MMAF/IMQ completely inhibited tumor growth with a final volume of ~0 mm³ on Day 7, and no recurrence was observed after stopping treatment during Day 15-29 (FIGS. 5A and 5C). The body weight profiles showed no difference between ADCs and saline groups (FIG. 5B). The IHC staining of TNBC PDX without treatment confirmed the positive expression of CD276 (FIG. 5D). These data indicated that our humanized CD276 mAb-directed ADCs can effectively target and treat TNBC PDX.

### In Vivo Anti-TNBC Efficacy of DualADC in TNBC Immunocompetent Models

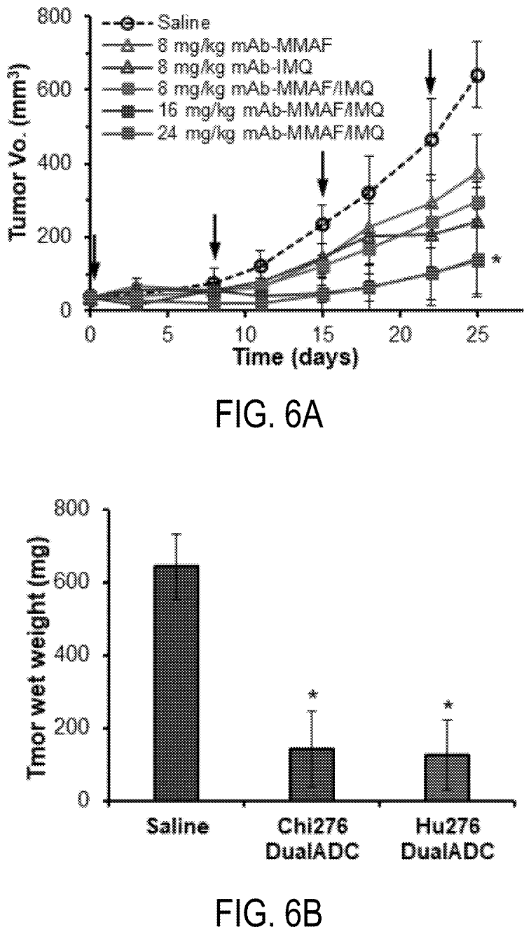

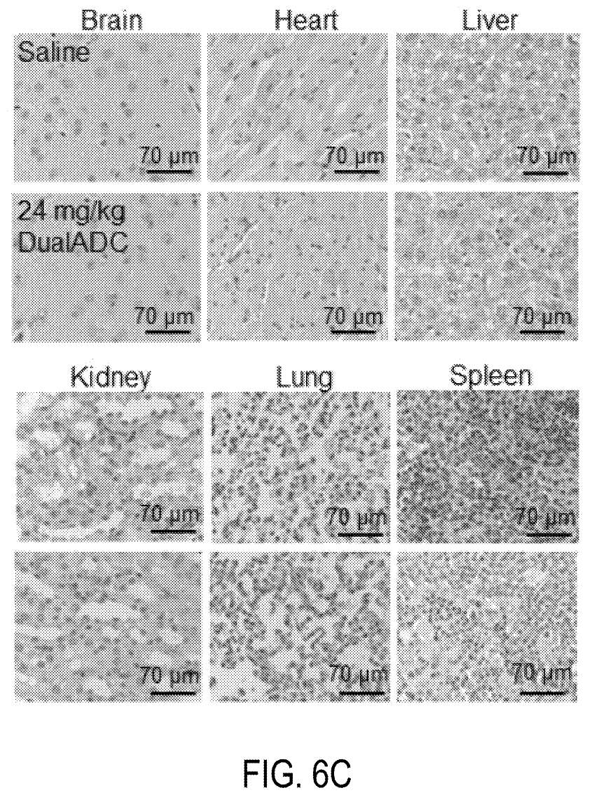

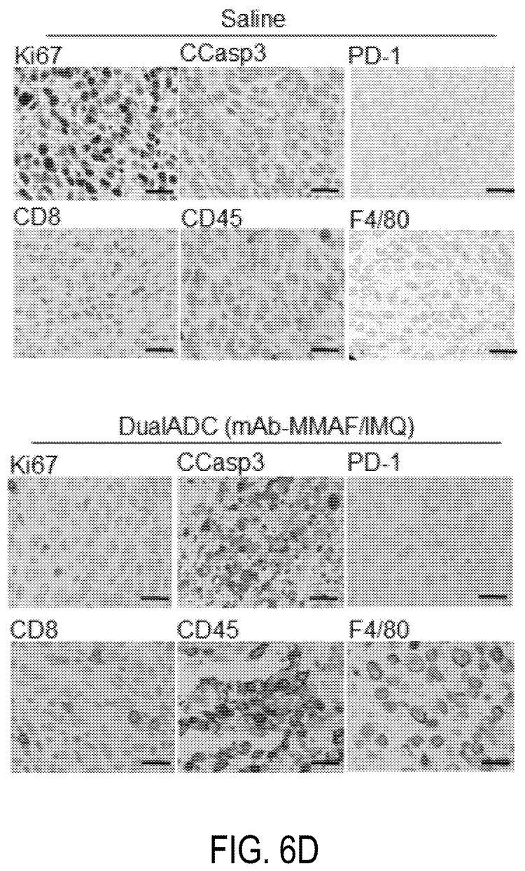

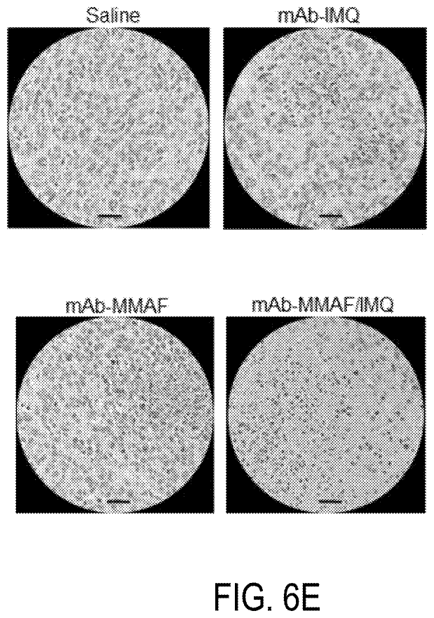

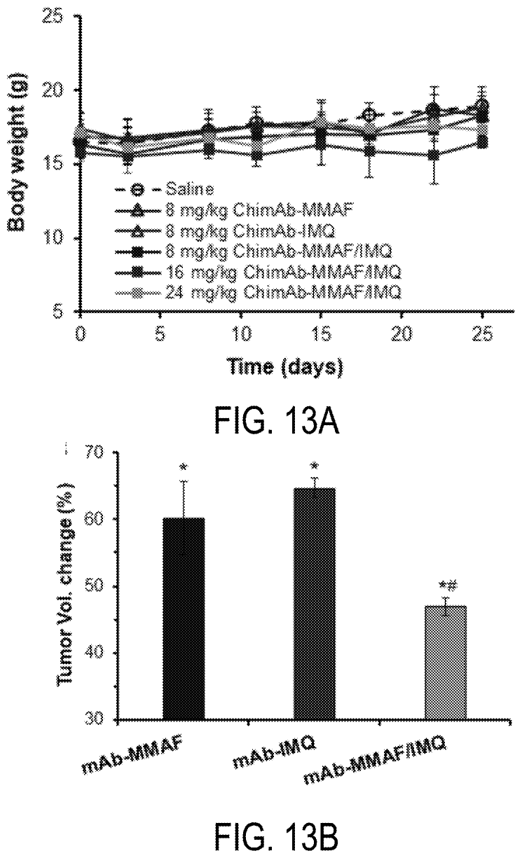

The synergistic chemo-immunotherapy of dual-payload ADC was further evaluated in 4T1-FLuc xenografted female BALB/cJ mouse models (n=5-8). It is found that 16 and 24 mg/kg of CD276 mAb-MMAF/IMQ reduced tumor burden to 142 mm³ 25 days after the 1^{st} injection, as compared to saline group with final tumor volume of 642 mm³ (FIG. 6A). The 8 mg/kg of mAb-MMAF/IMQ, mAb-MMAF and mAb-MMAF/IMQ showed medium treatment effect with final tumor volume of 241-376 mm³. Body weight had no obvious difference among the DualADC, singleADC and saline groups (FIG. 13). The two engineered mAbs (chimeric and humanized)-directed DualADCs (16 mg/kg) had similar anti-TNBC efficacy (FIG. 6B). Further pathology assessment with H&E staining of major organs (brain, heart, liver, kidney, lung, spleen) revealed no inflammation, apoptosis or necrosis, damage or toxicity in the 24 mg/kg DualADC group (FIG. 6C) although the enlarged spleen was observed at the endpoint.

### Anti-Cancer Mechanisms of Dual-Payload ADC

The underlying anti-cancer mechanisms were investigated with multiple research approaches, including H&E staining for tumor cell death, IHC staining with various antibodies to analyze tumoral immunity, Luminex assay to titrate tumoral cytokine secretion and test cytokine storm, whole blood analysis, and single-cell RNA sequencing (scRNA-Seq) to analyze immune cell infiltration, immune responses and mitotic activities in TME. All data suggested a combined chemo-immunotherapy of DualADC for TNBC treatment.

First, IHC staining of tumor tissues demonstrated obvious infiltration of activated cytotoxic CD8 T cells (CD8, CD45), activated NK cells (CD45), and phagocytosis of macrophage (F4/80) in the 24 mg/kg DualADC group (FIG. 6D). DualADC also reduced tumor intensity and slightly downregulated PD-1 expression. The expression of the proliferation marker (Ki67) was downregulated, and the apoptosis marker (CCasp3) was upregulated significantly in TNBC tumor treated with DualADC. These data indicated that our CD276-targeted DualADC mAb-MMAF/IMQ effectively modulated TNBC tumoral immunity. The H&E staining of tumor tissues showed healthy TNBC cells in the saline group, a certain level of cell death in single-payload ADC (mAb-MMAF, mAb-IMQ) groups, and severe cell death in DualADC (mAb-MMAF/IMQ) group (FIG. 6E).

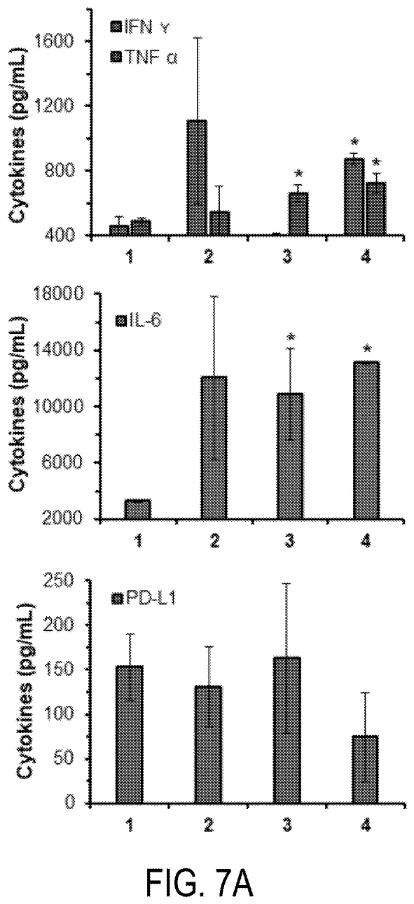

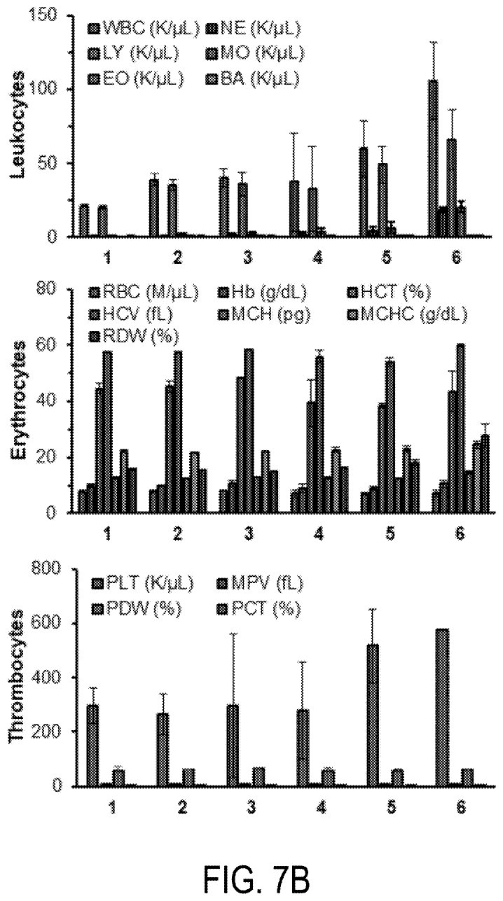

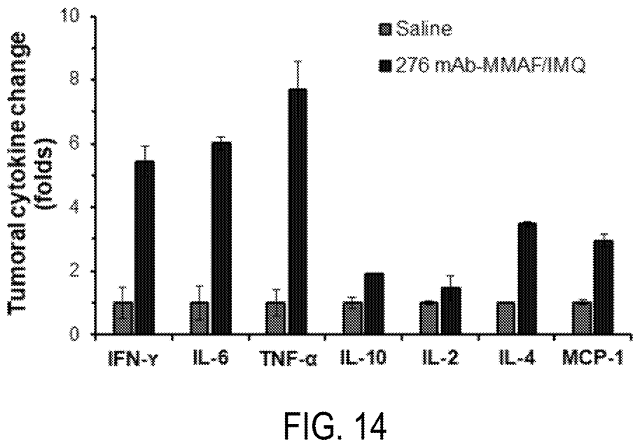

Second, Luminex assay of tumor tissues harvested at the end of treatment had several tumoral cytokines, such as TFN-γ, TNF-α and IL-6, with obvious enhancement, which confirmed the targeted delivery of IMQ by mAb and its immunotherapy in TNBC tumor (FIG. 7A). Additional tumoral cytokines (TFN-γ, TNF-α, IL-6, IL-10, IL-2, IL-4, MCP-1) profiles of TNBC from DualADC group were summarized in FIG. 14. These results confirmed that TLR 7/8 agonist boosted cytokine secretion in TME. About 4.2-6.0 μg of IMQ was detected in the TME (NOT cell lysis) of 1 mg of tumor samples at the end of the animal study, but an advanced analysis of the drug's dynamic distribution in tumor is needed in future studies.

Third, CBC analysis of whole blood samples showed neither mAb-MMAF and mAb-IMQ nor mAb-MMAF/IMQ significantly changed erythrocytes (red blood cell, hemoglobin, hematocrit) and thrombocyte (platelet, plateletcrit), as summarized in FIG. 7B. The leukocyte cell counts (white blood cell, neutrophil, lymphocyte, monocyte, eosinophils, basophile) of mice treated with 24 mg/kg of dual-payload ADC were higher than that treated with 16 mg/kg of DualADC. There was no anemia or blood clot observed during the treatment with ADCs. The chemocytokien analysis of serum samples demonstrated that most of the titrated 25 chemocytokines had no obvious differences among all groups except an increase of CCL4 in the mice treated with 16 and 24 mg/kg of DualADC and CXCLI in 24 mg/kg of DualADC group (Table 1). All these data indicated the safety and minimal systemic toxicity of the CD276-targeted mAb, single-payload ADCs, and DualADC with doses of 8-24 mg/kg.

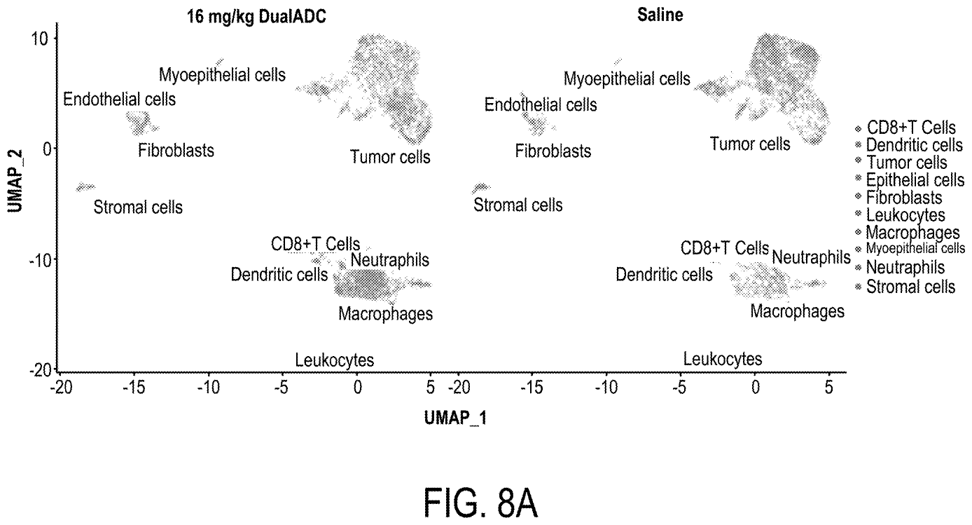

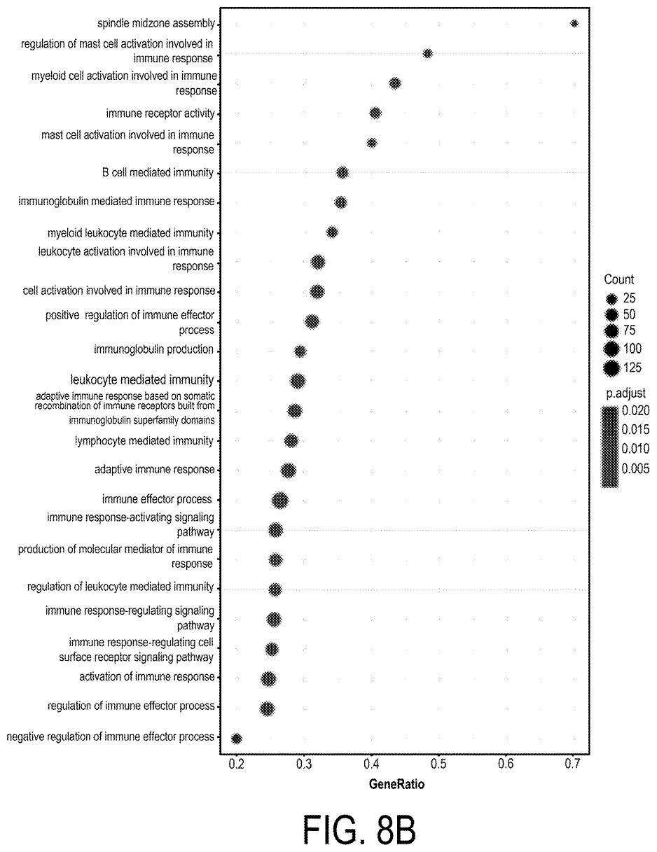

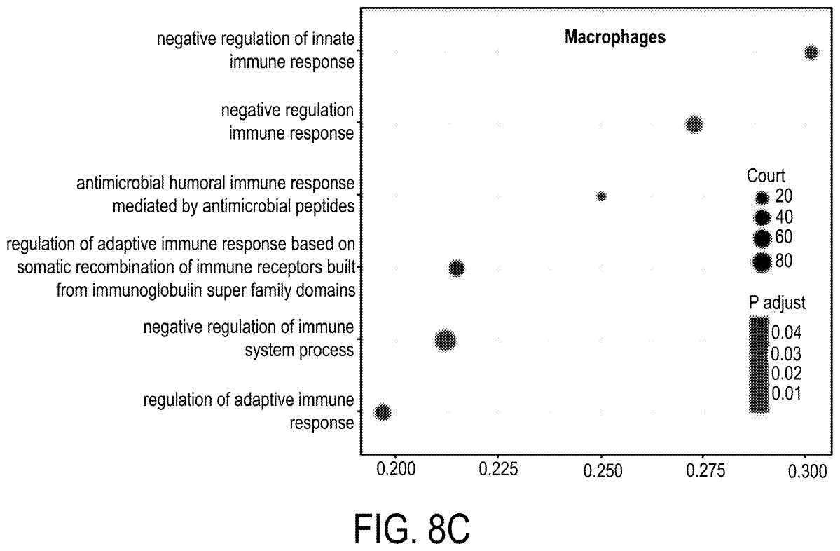

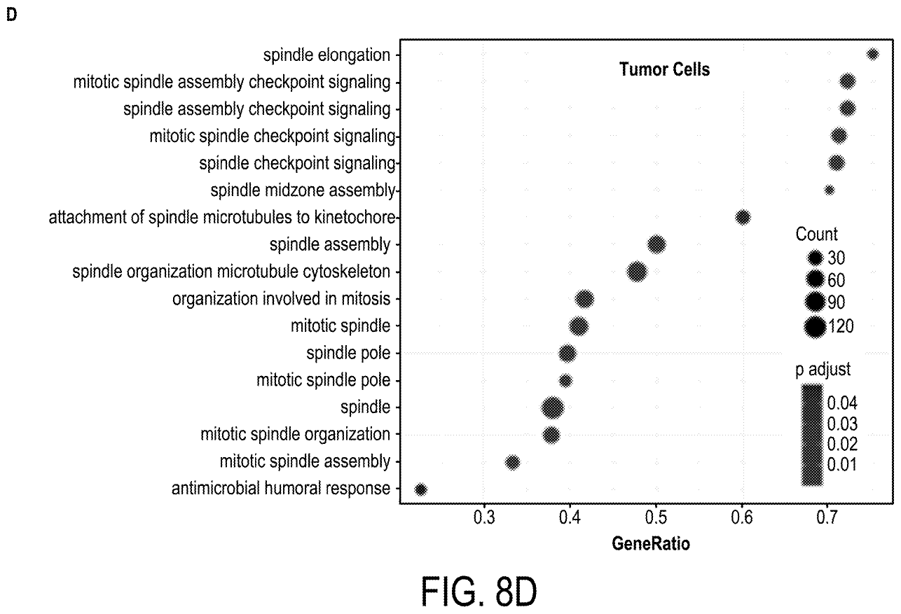

Finally, scRNA-Seq of TNBC identified and quantitated all cell types in tumor tissues, including tumor cells, myoepithelial cells, endothelial cells, fibroblasts, stromal cells, CD8⁺ T cells, dendritic cells, neutrophils, macrophages, and leukocytes (FIG. 8A). It was found that the percentage of immune cells infiltrated in the TME of 16 mg/kg DualADC group was 43.1%, much higher than 15.3% in saline group. These data were consistent with the IHC staining as presented in FIG. 6D but provided an accurate count of the immune cells. The counts and gene ratio of tumoral cells involved in various immune functions, such as spindle midzone assembly, mast cell activation, adaptive immune response, immune response-regulating cell surface receptor signaling pathway, activation of immune response, and immunity mediated by B cells, immunoglobulin, leukocyte, myeloid leukocyte and lymphocyte, were summarized in FIG. 8B. The immune regulation functions of macrophages (FIG. 8C), neutrophils, CD8⁺ T cells, dendritic cells, leukocytes, and other cells were observed in the tumor. The analysis of tumor cell distribution in different mitotic stages confirmed the MMAF caused inhibition of cell proliferation (FIG. 8D).

### Validation of Anti-TNBC Efficacy of DualADC in Immunocompromised Models

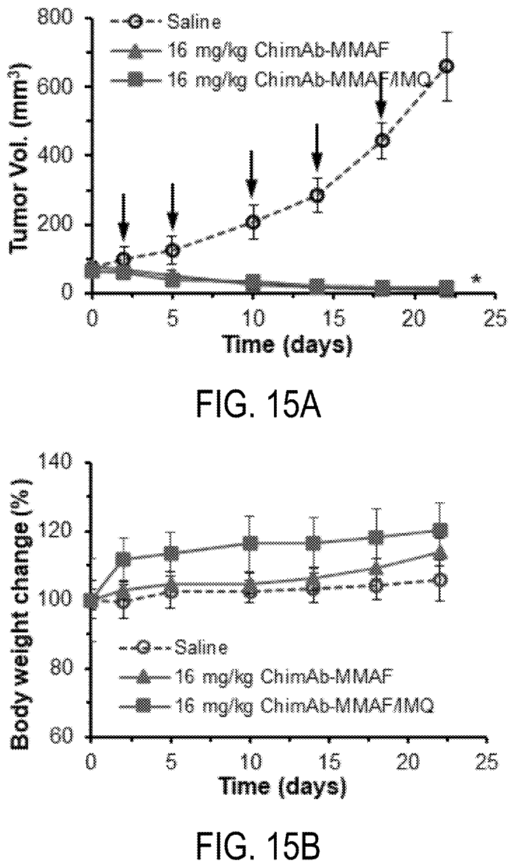

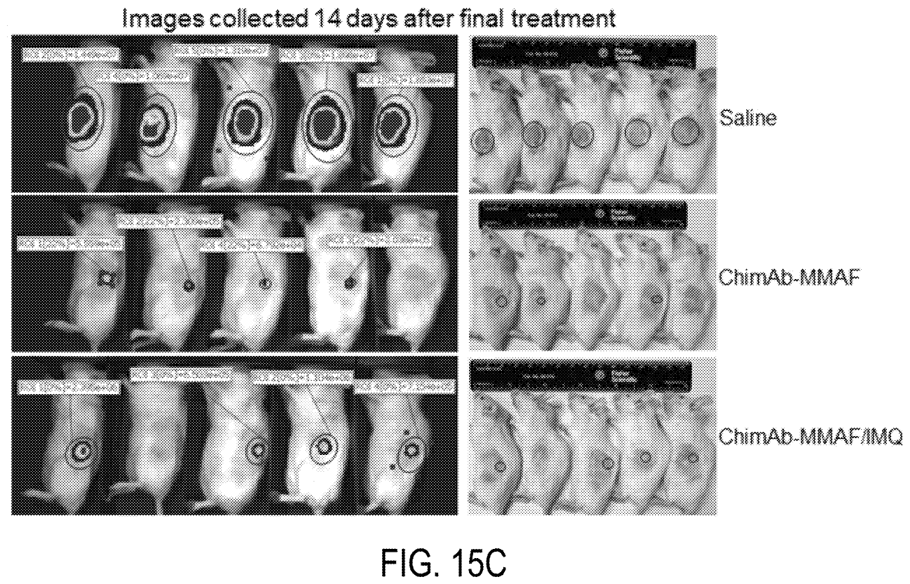

Anti-TNBC efficacy of 276 mAb-MMAF/IMQ was validated in immunocompromised models using MDA-MB-231-FLuc xenografted female NSG mice. FIG. 15A showed that TNBC tumor volume was reduced to 15-17 mm³ on Day 22 in 16 mg/kg of ADC treatment groups and 660 mm³ in saline group. No TNBC recurrence was observed 14 days after treatment was stopped. The change of body weight had no obvious difference among the treatment and saline groups (FIG. 15B). The endpoint IVIS imaging (FIG. 15C, left) and white light imaging (FIG. 15C, right) validated the high anti-TNBC efficacy of CD276-targeted ADC, e.g. reduced tumor volume and burden.

### Pharmacokinetics (PK)

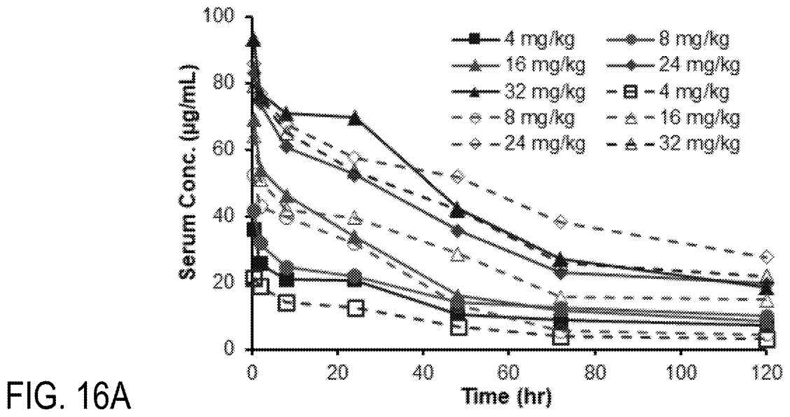

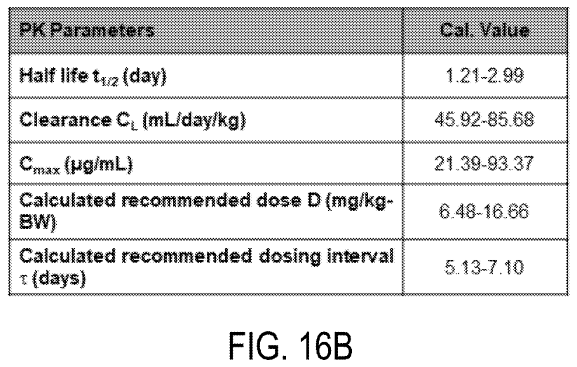

The PK parameters were assessed using 4T1 xenografted BALB/cJ models. The serum concentration profiles of CD276 mAb-MMAF/IMQ were presented in FIG. 16A. As summarized in FIG. 16B, t_{1/2}=1.21-2.99 days, Cₘₐₓ=21.39-93.37 μg/mL, D=6.48-16.66 mg/kg, and τ=5.13-7.10 days. None of the tested DualADC doses apparently affected mouse body weight, survival, and general health, including water intake, breathing and locomotion, while maximal toxicity dosage was not reached.

### Discussion

Targeted therapies, such as mAb, ADC and small molecule inhibitors, have been developed to treat solid tumors, but none has been offered to treat primary and metastatic TNBCs due to a lack of promising targets. This example confirmed the overexpression of CD276 receptor in the majority (over 60%) of TNBC patient tissues and multiple TNBC subtypes, consistent with TCGA transcript analysis and literature report of CD276 in 80% of breast cancers. Moreover, CD276 inhibits the immune functions of NK and T cells and reduces the secretion of effector cytokines as an immune checkpoint. This example developed an innovative CD276-targeted therapy by establishing a new platform to conjugate dual payloads with mAb, aiming to eliminate TNBC cells in vivo through synergistic anti-cancer mechanisms. The cross activity of CD276 mAb allowed us to evaluate the anti-TNBC efficacy and mechanism of DualADCs in mouse models. This therapy had high specificity to CD276⁺ tumor, effective tumor cell death, obvious immune cell infiltration and cytokine secretion in TEM. The tumor burden in all animal studies was significantly reduced, highlighting its great potential as a targeted therapy for TNBC treatment.

Different from traditional ADC using mAb to carry a single payload, our DualADC is comprised of CD276 mAb, a highly potent drug (MMAF), and an immune booster TLR agonist (IMQ). The concept of using DualADC to target and treat TNBCs (and other cancers) is innovative due to several advantages. First, DualADC combines different drugs with synergistic cancer therapeutic mechanisms, upregulating tumoral immunity while simultaneously inducing direct cancer cell death. The integration of chemotherapy and immunotherapy in one molecule could improve circulation stability, reduce side effects, and improve anti-cancer efficacy, especially for highly aggressive and heterogeneous cancers. Second, the established DualADC platform enables conjugating two different drugs at two sites (e.g., cysteine and lysine). Different from site-selective conjugation or attaching two payloads together, the conjugation strategy enables optimization of the ratio of two payloads. The cancer cells and immune cells might have different responses to the dual payloads, so the flexibility to adjust the ratio between two payloads could achieve optimal anti-cancer efficacy. In the future, there is a need to evaluate the effects of conjugation strategies, linkers, combination of different payloads, and the ratio of two payloads on circulation stability, potential toxicity and cancer treatment efficacy. Third, targeting CD276 could cover 60% of TNBC patients (and other CD276⁺ cancers), improve therapeutic efficacy by targeted delivery of drugs to tumor microenvironment, and reduce dose and dosage. Fourth, the engineered anti-CD276 mAbs could improve its plasma stability, biological function, and thereby translational potential, which need further evaluations in advanced animal models.

Importantly, the investigation of anti-cancer mechanisms of the DualADC using scRNA-Seq, Luminex assay, histology analysis, whole blood analysis and others identified multiple anti-TNBC mechanisms: 1) direct cancer cell killing and proliferation inhibition through potent payload, 2) combined tumoral immunity by CD276 mAb and TLR 7/8 agonist, and 3) TLR agonist-mediated tumoral cytokine enhancement.

First of all, the anti-CD276 mAb can effectively and specifically target the TNBC xenografts with minimal off-target and deliver payloads in vivo. Moreover, the clinical data and literature reports reported that blockade of CD276 can neutralize the inhibitory signaling, reactivate immune cells and restore effector immune functions. This study showed that anti-CD276 mAb reactivated the immune functions in TME by increasing the infiltration of activated NK and T cells. In addition to treating cancers as monotherapy, the combination of CD276 mAb with other therapies, such as enoblituzumab/retifanlimab and vobramitamab duocarmazine, shows great potential in preclinical or clinical studies. In the future, there will be further investigation of dually targeting CD276 and PD-1 with our unique ADCs in future studies to benefit the most (75-90%) TNBC cancer patients without response to ICBs due to primary resistance, acquired resistance, and relapse during treatment.

Pattern recognition receptor (PRR) agonists, such as TLR agonists, have been developed to treat cancer and other diseases, which target innate immune systems and stimulate immune responses via the MYD88 and other pathways. PRR agonists, including TLR 7/8/9 agonists, RIG-I, MDA-5, and STING, have been investigated in preclinical studies or clinical trials. Administration of free TLR agonists which lack tumor selectivity, may cause fatal cytokine storm and other side effects. The conjugation linkers and strategies developed in this study enable delivering TLR agonists to tumors directly, overcoming the challenge of systematic toxicity. Consistent with the literature, the present example shows that TLR 7/8 agonists, targeting delivered with our anti-CD276 mAb, inhibit TNBC cancer proliferation, induce tumor cell apoptosis, and stimulate the production of multiple cytokines by immune cells that are activated in TME. In addition, the tumor-associated antigen released by dead tumor cells can lead to a cascade of adaptive immune responses through antibody-dependent cellular phagocytosis.

In addition to immunotherapy functions, both potent payload (MMAF) and immune boosting reagent (IMQ) showed cytotoxicity to TNBC cells. MMAF can effectively block microtubulin polymerization and inhibit the mitotic process and proliferation of TNBC cells. This study confirmed the cytotoxicity and direct cell death of free MMAF and single-payload ADC (mAb-MMAF). In addition to upregulating immune function, the TLR 7/8 agonist and single-payload ADC (mAb-IMQ) also showed cytotoxicity to TNBC cells by inhibiting proliferation, reducing survival and increasing apoptosis. The combination of MMAF and IMQ in one DualADC is more efficient in killing TNBC cells by integrating different anti-cancer mechanisms, which has great potential to bypass drug resistance, overcome cancer recurrence, and improve survival. Alternatively, the heterogenous TNBCs with low CD276 expression could be targeted with other mAbs via alternative receptors such as EGFR, Trop-2, LSR or GRP56 as combined therapies, which could be investigated in future studies.

This example demonstrated high plasma stability of the DualADC in PK study and high TNBC specificity in biodistribution study. These features could benefit the TNBC treatment efficacy in future pre-clinical or clinical studies. Although MTD was not reached, all tested doses of DualADC did not indicate toxicity in major organs, blood cell count, serum cytokine, body weight, survival, and general health. A future Maximal Tolerated Dosage (MTD) study is needed to define the safe dose for cancer treatment. A toxicology study is also highly desired to fully evaluate the potential toxicity including the effect on antigen-presenting cells with low CD276 expression.

In summary, this example developed an innovative therapy, CD276-targeted dual-payload antibody-drug conjugate, which combines chemotherapy and immunotherapy into one molecule aiming to eliminate the aggressive and heterogeneous TNBCs. The promising anti-TNBC efficacy and minimal side effects were validated in multiple mouse models and post treatment analyses. The concept of targeted delivery of dual payloads with synergistic functions is novel and readily translationable.

### Example 2: MAb and ADC Purification Using Liquid Chromatography (LC)

mAb: monoclonal antibody
ADC: antibody-drug conjugate
LC: liquid chromatography
Columns selection for one-step or two-step purification:
Primary purification: UNOsphere SUPrA Cartridge (affinity) and other Protein A columns
Intermediate and Polish: Nuvia S (cation exchange)

TABLE 2
Step 1. Primary Purification - Protein A (UNOsphere SUPrA, affinity)

| | | Column | Flow |
|---|---|---|---|
| Step | Buffer | volume | rate |
| Equilibrate A1 | 0.02M sodium phosphate, 0.02M sodium | 10-15 | 5 mL/min |
| | citrate, pH 7.5 (A1) | | (140 |
| | | | cm/hr) |
| Sample | diluted 1:10 into buffer A1; | — | 5 mL/min |
| loading | adjust to pH 7.5 with phosphoric acid or NaOH; | | |
| | clarified with 0.2 um filter | | |
| Wash Al | A1 | until | 5 mL/min |
| | | absorbance | |
| | | returns to | |
| | | baseline | |
| Elute*, from | 100% Buffer B1: | 10 | 5 mL/min |
| Al to B1 | 0.02M sodium citrate, 0.1M sodium chloride, | | |
| | pH 3.0 (good for most mAb) | | |
| Strip B1 | Buffer B1 | 5 | 5 mL/min |

*Elute fractions collected.

TABLE 3
Step 2. Intermediate/Polishing Purification - Ion Exchange (Foresight
Nuvia S, cation exchange)

| | | Column | |
|---|---|---|---|
| Step | Buffer | Volume | Flow rate |
| Equilibrate | 20 mM CH3COONa, 20 mM NaCl, | 15 | 2.5 mL/min |
| | pH = PI-1.0 (or 1.5) (Buffer A2) | | (297.5 |
| | PI of mAb1 is 8.45, can use HEPES | | cm/hr) |
| | buffer at pH 7, or MOPS at a pH of | | |
| | around 6.8 to 7 | | |
| Sample | Eluted from capture, diluted 1:5 with | — | 2.5 mL/min |
| loading | dH2O, adjusted to pH = PI-1.0 (or | | |
| | 1.5) with 1M phosphoric acid or base. | | |
| Wash | Buffer A2 | until | 2.5 mL/min |
| | | absorbance | |
| | | returns to | |
| | | baseline | |
| Elute* | Buffer B2 | 15 | 2.5 mL/min |
| | 20 mM sodium acetate, 200 mM | | |
| | NaCl, pH 4.9 (too low, PI-1.0 or 1.5). | | |

*Elute fractions collected.
Note:
Check Isoelectric Point of humanized antibody.

### Steps:

1. mAb/ADC purification procedure-Protein A column:

Note: If two-step purification is needed, please follow the equilibration, loading, wash, and elute in Step 2 as detailed in the Table 2.

1.1 Equilibrate column: with 10 CV buffer A1.

1.2 Inject mAb sample: mAb sample either as is or diluted 1:10 or 1:5 into buffer A1.

1.3 Wash column: buffer A1 until absorbance returns to baseline, or 5-6 column volume.

1.4 Elute mAb: in a 10 or 5 CV linear gradient to 100% B, or desired % buffer B. Eluate mAb in neutralization buffer.

1.5 Strip: with 5 CV buffer B.

1.6 Desalt and formulate purified mAb: Use PD SpinTrap™ G-25 Desalting Column to remove salt or dialysis for buffer exchange immediately after LC/Protein A purification. Adjust pH to match the formulation buffer. Add 10% of 10× formulation buffer for long-term storage at −20° C. or 4° C. Note: need to optimize formulation buffer and pH.

Titrate mAb using nanodrop and check purity/quality using SDS-PAGE.

### Notes:

Buffer A1 was used for binding human and guinea pig IgG. Buffer A2 was used for all others.

The recommended column equilibration interval was excessive under most conditions but was used as a default until specific equilibration requirements are established.

In addition, caution was taken of solubility limitations of antibodies that require high salt concentrations (A2) for binding. If the antibody failed to remain fully soluble for the longest possible duration from equilibration to completion of sample load when adjusted to load conditions, then load by using online dilution technique.

Initial selectivity screening was conducted with a linear gradient.

The choice of citrate for the low pH buffer is predicated on the broad pH range achievable with phosphate/citrate systems. If a higher pH range is required, add boric acid to the binding buffer.

### Cleaning LC System (Every 6 Months or as Needed)

To ensure that all bound substances were released and washed out of the column, the following cleaning-in-place (CIP) protocols were recommended. The following protocols are suggested to remove precipitated or denatured substances from the bed.

2.1 Wash the bed with 2-5 column volumes in reverse flow with one of the following solutions:
6M guanidine hydrochloride
10 mM hydrochloric acid
0.1M sodium hydroxide
1M acetic acid/20% ethanol
   2.2 Followed by a reverse flow wash with at least 5 column volumes of binding buffer (e.g. Buffer A1), neutral pH (7-8).
   2.3 To remove any hydrophobically bound substances from the bed, wash the column with 2-5 column volumes in reverse flow of a nonionic surfactant/detergent, followed by a reverse-flow wash with at least 5 column volumes of neutral pH binding buffer.
   2.4 Suggested contact time per cycle is 15 min at room temperature.

### Notes:

Sanitization (as needed)—To reduce the potential for microbial contamination of the cartridge, the column can be periodically washed with a solution consisting of 0.1M sodium hydroxide. Allow to stand for 1 hour, then wash with buffer until a neutral pH is reached.
Column Storage—To store UNOsphere SUPrA for long periods, equilibrate the media with a 20% ethanol/water solution and store at 4° C.
Preparing a column for use—All columns are ready to use after equilibrating the columns win the buffer of choice. To perform a buffer exchange, connect the column to a liquid chromatography system or peristaltic pump and condition it as follows:
Set the pump flow rate to 0.5-1 mL/min (59.5-119 cm/hr) for the 1 mL column or 2.5-5 mL/min (297.5-595 cm/hr) for the 5 mL column.
Wash the column with degassed low-salt buffer for 2 column volumes. Wash with degassed high-salt buffer for 5 column volumes.
Equilibrate with low-salt buffer for 5 column volumes.
Reduce the flow rate to the rate that will be used in the purification protocol.

### Sample Preparation

Correct pH and ionic strength were necessary for consistent and reproducible results. Sample was exchanged into the starting buffer or diluted to the starting buffer concentration. This was achieved by diluting the sample to the ionic strength of the starting buffer, dialyzing against the starting buffer, or exchanging into the starting buffer. All samples were filtered through a 0.45 μm filter prior to column application.

### Cleaning-In-Place and Sanitation

If a column no longer yielded reproducible results, the media may have required thorough CIP and sanitation to remove strongly bound contaminations. Acceptable CIP agents include 25% acetic acid, 8 M urea, 1% Trition X-100, 6 M potassium thiocyanate, 70% ethanol, 30% isopropyl alcohol, 1 N NaOH, and 6 M guanidine hydrochloride.
1. Sanitize the support in the column with 2-4 bed volumes of 1.ON NaOH while maintaining a contact time of at least 40 min.
2. To re-equilibrate the column, wash it with 2-4 bed volumes of 0.5-2 M NaCl solution (containing 50-100 mM buffer).
3. If lipid removal is required, wash the column with a 20-70% ethanol solution.

### Example 3: Dual-Payload ADC

### Anti-Cancer Mechanisms of Dual ADC

FIG. 1 shows synergetic mechanisms of anti-CD276 mAb-MMAF (DM1)/TLR 7/8 agonist to treat TNBCs, wherein 1. shows DualADC targeting and binding to the surface receptor CD276 of TNBC cells, 2. shows internalization, 3. shows drug release, 4. shows MMAF-induced inhibition of microtubule polymerization, 5. shows TLR 7/8 agonist-induced cytotoxicity, and 6. Shows immune cells activation by anti-CD276 mAb and TLR 7/8 agonist.

### DualADC Construct 1

FIGS. 3A-3E show construction of DualADC via cysteine and lysine. FIG. 3A shows structure of DualADC CD276 mAb-MMAF/IMQ. FIG. 3B shows structure of DaulADC. FIG. 3C shows HPLC confirmed the conjugation of single payload and dual payloads with chimeric anti-CD276 mAb. FIG. 3D shows MALDI-TOF MS to validate the right molecular weight of mAb, single ADC (mAb-MMAF, mAb-IMQ) and DualADC (mAb-MMAF/IMQ). FIG. 3E shows SDS-PAGE of ADCs. M: Marker; 1: CD276 mAb; 2: mAb-MMAF; 3: mAb-IMQ; 4: mAb-MMAF/IMQ.

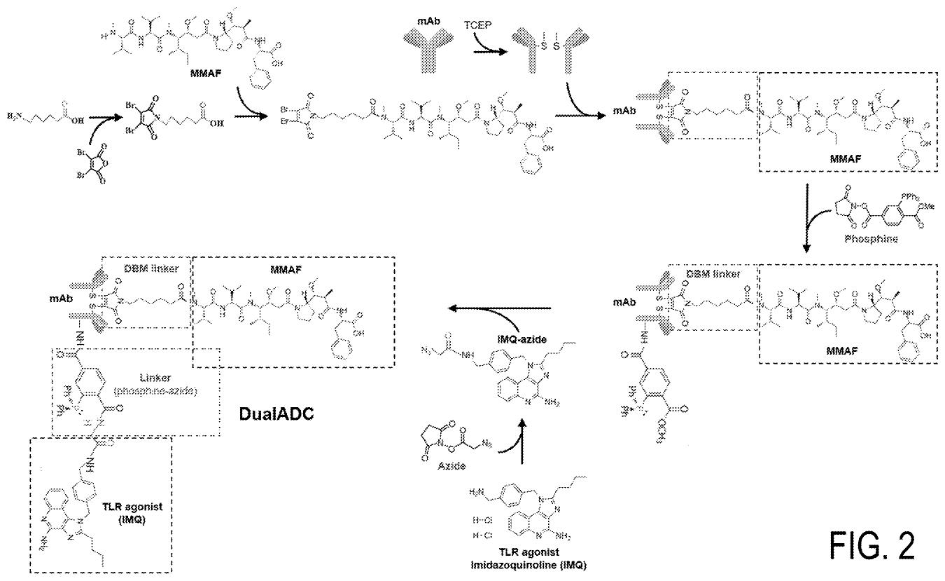

The synthesis pathway for DualADC Construct 1 is shown in FIG. 2. In the figure, the steps resulting in conjugation of the DBM linker occur first and subsequently, the phosphine and azide are conjugated to the agonist. In some examples, the resulting phosphine-azide linker is conjugated first and the DBM linker results from the second conjugation step. Conjugation can happen in either order. Furthermore, phosphine and azide can be conjugated to the antibody and drug in either order (e.g., conjugation of phosphine first and then azide second or azide first and then phosphine second). The concept of conjugating a potent payload, such as a chemotherapy drug like MMAF, and an immune booting reagent with one monoclonal antibody is notable and allows for the superior anti-cancer efficacy of this DualADC when compared to earlier antibody drug conjugates.

### DualADC Construct 2

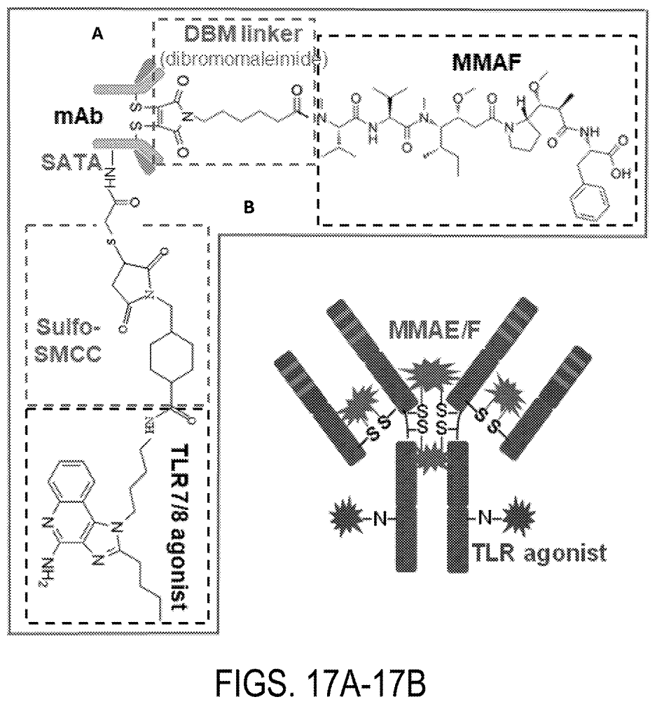

FIGS. 17A and 17B show construction of an example Dual ADC via cysteine and lysine. FIG. 17A shows a corresponding schematic of the DualADC comprising a DBM linker and a phosphine-azide linker. FIG. 17B shows an example chemical structure of a DualADC comprising a DBM linker and a sulfo-SMCC linker.

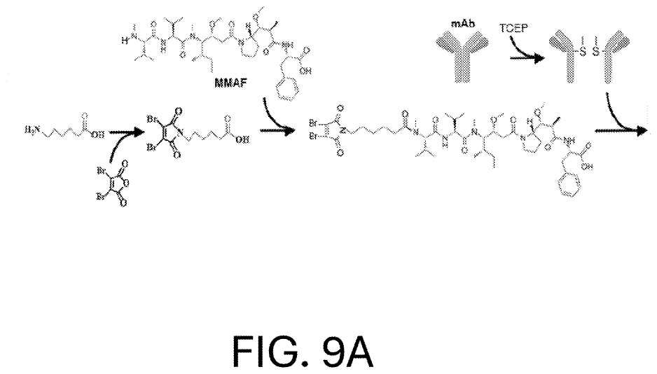

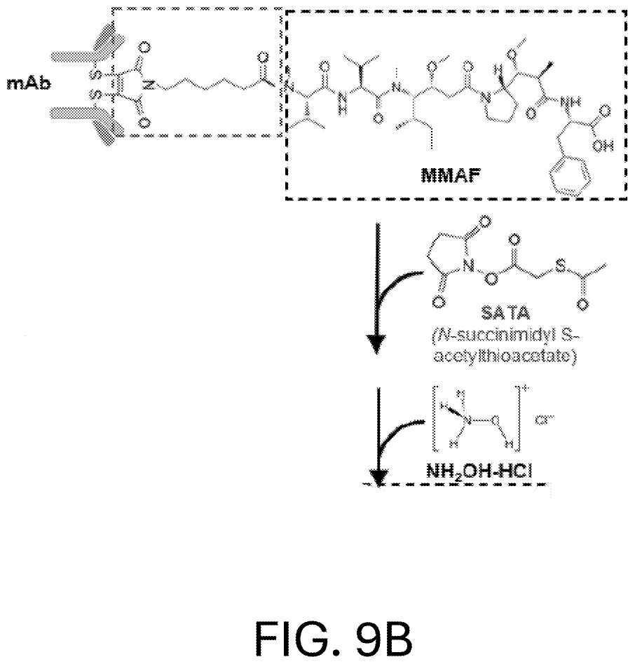

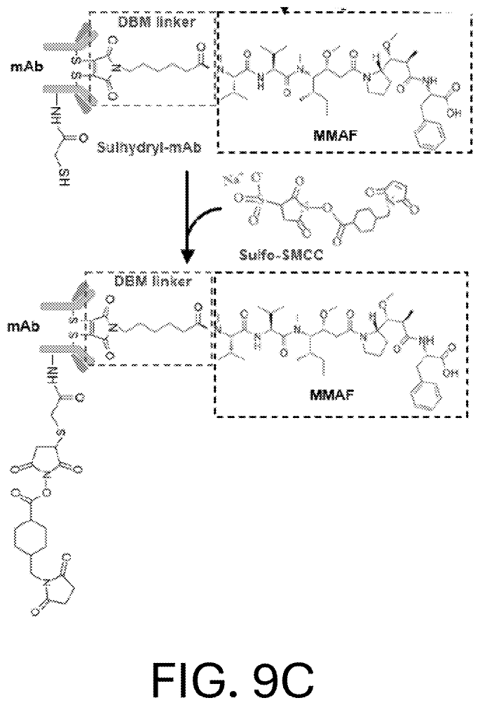

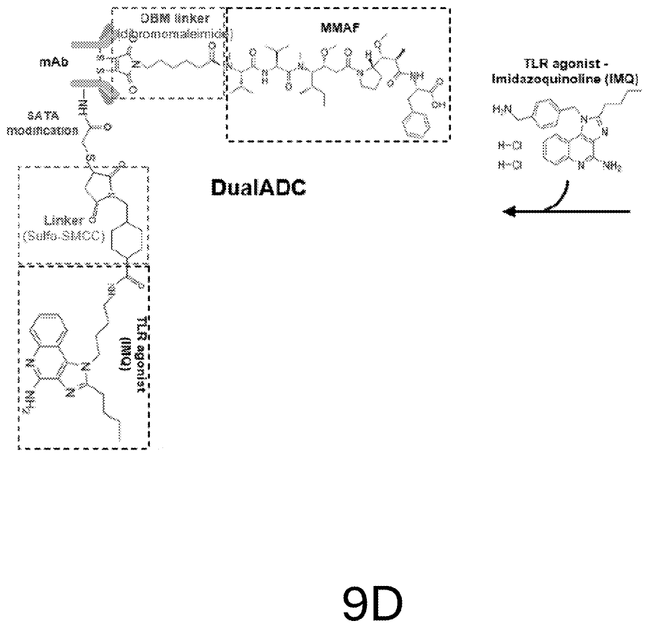

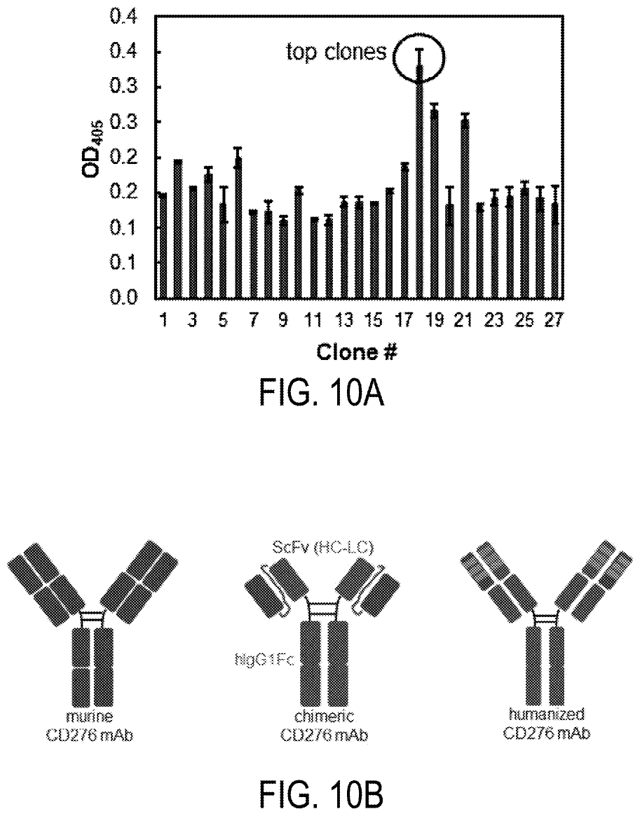

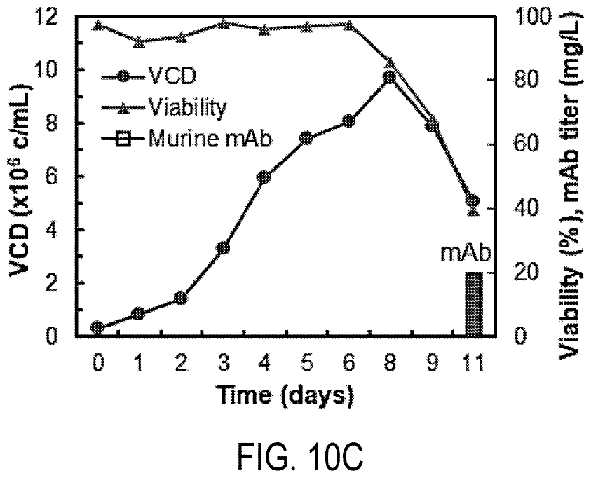

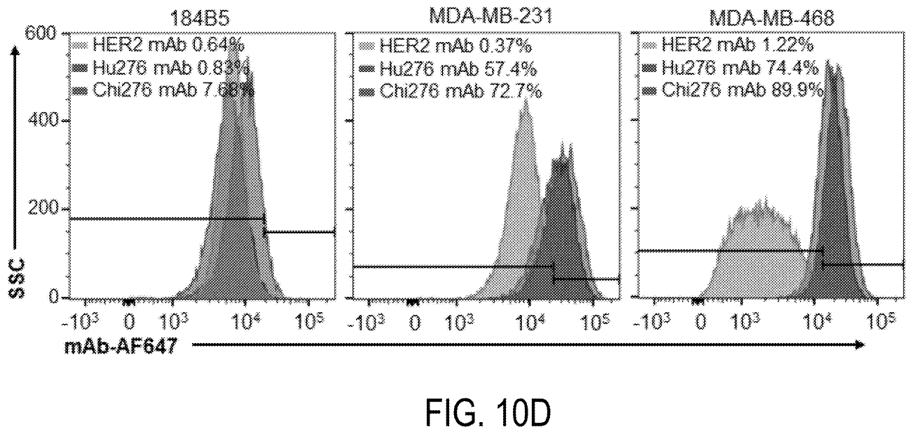

The synthesis pathway for DualADC Construct 2 is shown in FIG. 9. In the figure, the steps resulting in conjugation of the DBM linker occur first and subsequently, the SATA and sulfo-SMCC are conjugated to the agonist. In some examples, the resulting SATA sulfo-SMCC linker is conjugated first and the DB linker results from the second conjugation step. Conjugation can happen in either order. Furthermore, SATA and sulfo-SMCC can be conjugated to the antibody and drug in either order (e.g., conjugation of SATA first and then sulfo-SMCC or sulfo-SMCC first and then SATA second). The concept of conjugating a potent payload, such as a chemotherapy drug like MMAF, and an immune booting reagent with one monoclonal antibody is notable and allows for the superior anti-cancer efficacy of this DualADC when compared to earlier antibody drug conjugates.

### Single-Payload ADCs Construct

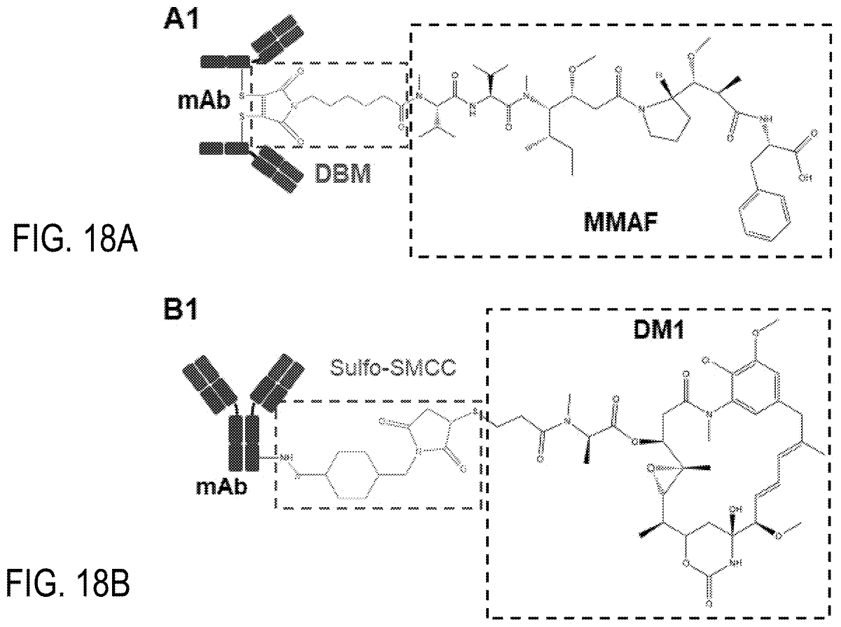

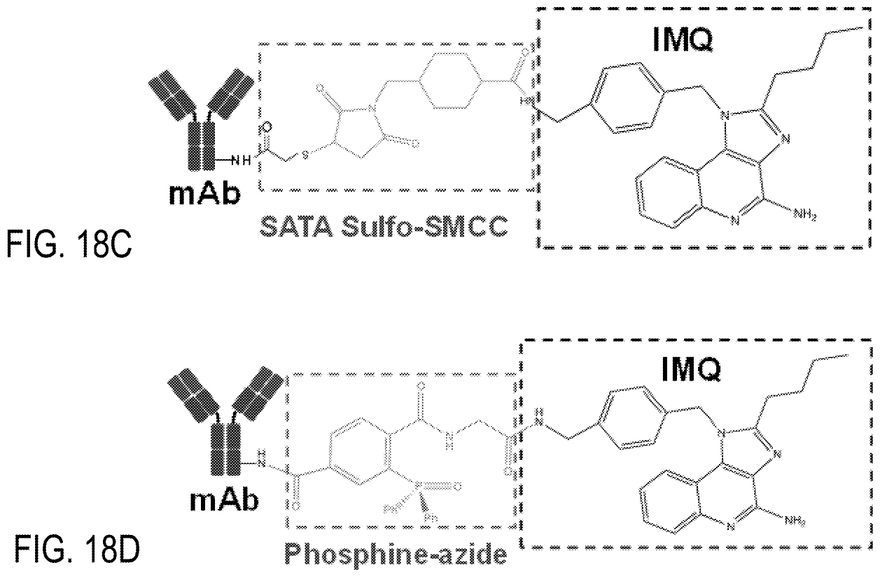

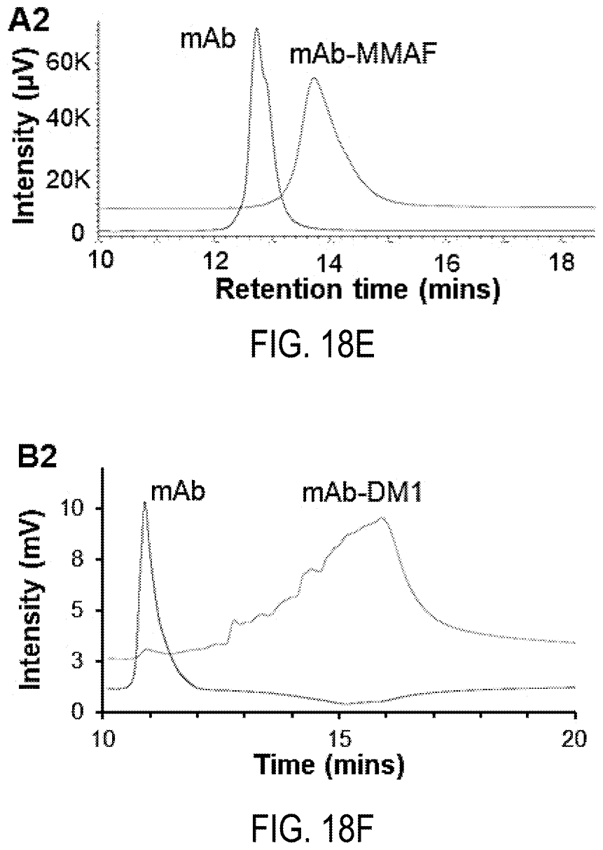

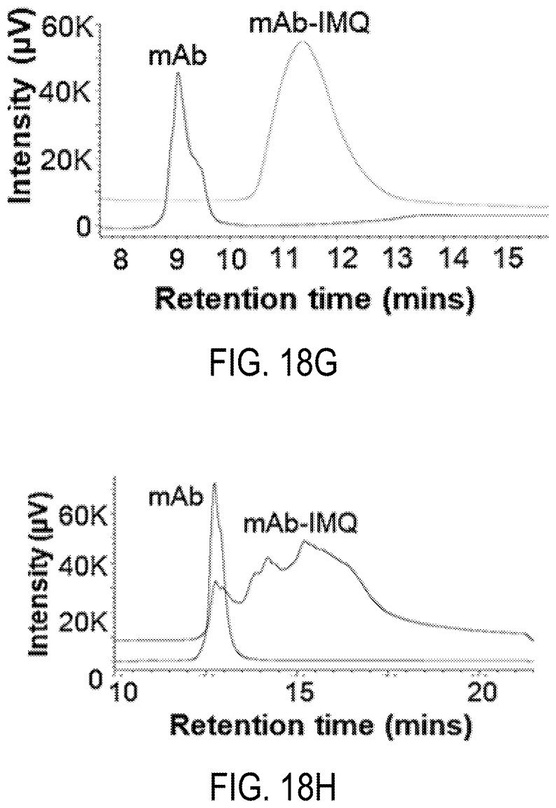

FIGS. 18A-18H show construction of single-payload ADC via lysine or cysteine. FIG. 18A show mAb-MMAF conjugate via lysine using bridging linker of DBM. FIG. 18B shows mAb-DM1 conjugate via lysine using linker of Sulfo-SMCC. FIG. 18C shows mAb-TLR agonist conjugate via lysine using linker of Sulfo-SMCC and SATA modification. FIG. 18D shows mAb-TLR agonist conjugate via synthesized linker of phosphine-azide. FIGS. 18E-18H are ADC (and mAb) characterizations using HPLC corresponding to FIGS. 18A-18D, respectively.

### In Vitro Cytotoxicity

FIGS. 4A-4F show in vitro evaluations of DualADC using humanized CD276 mAb. The TNBC MDA-MB-231, MDA-MB-468 and 4T1 cells are used in cytotoxicity studies with free drugs and single-payload ADC as controls. FIG. 4A shows anti-TNBC cytotoxicity and IC₅₀ of free DM1 and MMAF drugs. FIG. 4B shows cytotoxicity and IC₅₀ of IMQ. FIG. 4C shows EC₅₀ of IMQ on human and murine TLR8⁺ HEK cells. FIG. 4D shows anti-TNBC cytotoxicity and IC₅₀ of single-payload ADC (mAb-MMAF). FIG. 4E shows cytotoxicity and IC₅₀ of single-payload (mAb-IMQ). FIG. 4F cytotoxicity and IC₅₀ of DualADC mAb-MMAF/IMQ.

Anti-TNBC with DualADC in TNBC PD Models

FIGS. 5A-5D show anti-tumor efficacy of DualADC in TNBC PDX xenograft model. FIG. 5A shows tumor volume changes post treatment with humanized CD276 mAb-derived ADC following the schedule of Q7Dx3 as indicated by the black arrow. Data were presented as mean±SEM, n=5-7. Saline (◯), 16 mg/kg of mAb-MMAF (▴), and 16 mg/kg of mAb-MMAF/IMQ (●). *P<0.05 vs. saline using ANOVA followed by Dunnett's t-test. FIG. 5B shows body weight. FIG. 5C shows white light images at 14 days after treatment stopped. FIG. 5D shows IHC staining of TNBC PDX tumor tissue. The scale bar equals 20 μm.

### Anti-TNBC by Dual ADC in TNBC Immunocompetent Models

FIGS. 6A-6E shows anti-TNBC efficacy of 276 mAb-MMAF/IMQ in immunocompetent models. The mouse TNBC 4T1-FLuc xenografted female BALB/cJ mice were treated with DualADC (8, 16, 24 mg/kg mAb-MMAF/IMQ), mAb-MMAF, mAb-IMQ, or saline (controls) via i.v. injection through tail vein. n=5-8. FIG. 6A shows tumor volume post treatment following schedule of Q7Dx4 as indicated by black arrow. Data were presented as mean±SEM. *P<0.05 vs. saline using ANOVA followed by Dunnett's t-test. FIG. 6b shows weight of terminal wet tumors treated with 16 mg/kg of DualADCs using chimeric CD276 mAb and humanized 276 mAb. FIG. 6C shows H&E staining of major organs. Scale bar equals 70 μm. FIG. 6D shows IHC staining of harvested tumors using markers of cell proliferation (Ki67), apoptosis (CCasp3), immune checkpoint inhibition (PD-1), and filtration and activation of CD8⁺ T, NK and macrophage cells (CD8, CD45, F4/80). Scale bar equals 20 μm. FIG. 6E shows HE staining to analyze TNBC cell death in treatment group. Scale bar equals 40 μm.

### Compare Single and Dual Payload ADCs

FIG. 13 shows profiles of body weight of immunocompetent models treated with DualADC. The 4T1-FLuc xenografted female BALB/cJ mice were treated with dual-payload ADC (8, 16, 24 mg/kg mAb-MMAF/IMQ), mAb-MMAF, mAb-IMQ, or saline (controls) via i.v. injection through tail vein. n=6-8.

### Tumoral Immunity

FIGS. 7A-7B show analysis of tumoral cytokines and general toxicity post treatment of dual-payload ADC. The same mice as in FIG. 6 were used here. (A) Luminex assay identified several enhanced cytokines and downregulated PD-1 in TME. (B) The complete blood cell counts. 1: Saline (control); 2:8 mg/kg mAb-MMAF (control); 3:8 mg/kg mAb-IMQ (control); 4:8 mg/kg mAb-MMAF/IMQ; 5:16 mg/kg mAb-MMAF/IMQ; 6:24 mg/kg mAb-MMAF/IMQ.

### SCRNA-Seq of DualADC Treated TNBC

FIGS. 8A-8D show analysis of immune cell infiltration and immune functions in TME using single-cell RNA sequencing (scRNA-Seq). The tumor tissues were harvested from the same animal study in FIG. 6. FIG. 8A shows overview of all cell types in TNBC tumors. FIG. 8B shows immune functions in TME. FIG. 8C shows immune responses of macrophage. FIG. 8D shows analysis of mitotic activities.

### DualADC in Immunocompromised Models

FIGS. 15A-15C show anti-TNBC efficacy of dual-payload ADC (chimeric CD276 mAb-MMAF/IMQ) in immunocompromised models. The MDA-MB-231-FLuc xenografted female NSG mice were treated with dual-payload ADC (16 mg/kg mAb-MMAF/IMQ), single-payload ADC (16 mg/kg mAb-MMAF), and saline (control) via i.v. injection through the tail vein. n=5. FIG. 15A shows tumor volume post treatment following schedule of Q5Dx5 as indicated by the black arrows. Tumor volume was measured with calipers and calculated as ellipsoid. Data were presented as mean±SEM. *P<0.05 vs. saline using ANOVA followed by Dunnett's t-test. FIG. 15B shows profiles of body weight change. FIG. 15C shows IVIS bioluminescence and white light images at 14 days after the final treatment injection.

Pharmacokinetics—CD276 mAb-MMAF/IMQ

FIGS. 16A-16B show pharmacokinetics (PK) study. Anti-CD276 mAb-MMAF/IMQ in BALB/cJ mouse models. 6 dosages, n=2, total of 12 mice. FIG. 16A shows serum titer of DualADC. FIG. 16B shows PK parameters.

Other advantages which are obvious, and which are inherent to the invention, will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

Sequences

SEQ ID NO: 1 (Signal peptide of heavy chain)
MGWSYIILFLVATATGVH
SEQ ID NO: 2 (Murine framework region 1 (FR1) of heavy chain)
QVQLVQSGAEVKKPGASVKVSCKAS
SEQ ID NO: 3 (Murine FR2 of heavy chain)
MHWVRQAPGQGLEWMGS
SEQ ID NO: 4 (Murine FR3 of heavy chain)
YNQKFKDRVTMTRDTSISTAYMELSRLRSDDTAVYYC
SEQ ID NO: 5 (Murine FR4 of heavy chain)
WGQGTLVTVSS
SEQ ID NO: 6 (Human complementarity determining region 1 (CDR1) of heavy chain)
GYTFTEYT
SEQ ID NO: 7 (Human CDR2 of heavy chain)
NNPNTGGTT
SEQ ID NO: 8 (Human CDR3 of heavy chain)
SRSGNDVGWYFAV
SEQ ID NO: 9 (Constant region of heavy chain)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 10 (Chimeric heavy chain)
MGWSYIILFLVATATGVHSQVQLVQSGAEVKKPGASVKVSCKASGYTFTEYTMHW
VRQAPGQGLEWMGSNNPNTGGTTYNQKFKDRVTMTRDTSISTAYMELSRLRSDDTA
VYYCSRSGNDVGWYFAVWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHE
ALHNHYTQKSLSLSPGK-
“-” indicates a stop codon.
SEQ ID NO: 11 (Signal peptide of light chain)
MDMRVPAQLLGLLLLWLSGARC
SEQ ID NO: 12 (Murine FR1 of light chain)
EIVMTQSPATLSVSPGERATLSCTAT
SEQ ID NO: 13 (Murine FR2 of light chain)
MHWYQQKPGQAPRLLIY
SEQ ID NO: 14 (Murine FR3 of light chain)
KLASGIPARFSGSGSGTEFTLTISSLQSEDFAVYYC
SEQ ID NO: 15 (Murine FR4 of light chain)
FGGGTKLEIKR
SEQ ID NO: 16 (Human CDR1 of light chain)
SSVSY
SEQ ID NO: 17 (Human CDR2 of light chain)
DTS
SEQ ID NO: 18 (Human CDR3 of light chain)
QQWSSNPLT
SEQ ID NO: 19 (Constant region of light chain)
TVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
SEQ ID NO: 20 (Chimeric light chain)
MDMRVPAQLLGLLLLWLSGARCEIVMTQSPATLSVSPGERATLSCTATSSVSYMHW
YQQKPGQAPRLLIYDTSKLASGIPARFSGSGSGTEFTLTISSLQSEDFAVYYCQQWSSN
PLTFGGGTKLEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPVTKSF
NRGEC-
“-” indicates a stop codon.
SEQ ID NO: 21 (Signal peptide of heavy chain)
MGWSYIILFLVATATGVHS
SEQ ID NO: 22 (Murine framework region 1 (FR1) of heavy chain)
QVQLVQSGAEVKKPGASVKVSCKAS
SEQ ID NO: 23 (Murine FR2 of heavy chain)
MHWVRQAPGQGLEWMGS
SEQ ID NO: 24 (Murine FR3 of heavy chain)
YNQKFKDRVTMTRDTSISTAYMELSRLRSDDTAVYYC
SEQ ID NO: 25 (Murine FR4 of heavy chain)
WGQGTLVTVSS
SEQ ID NO: 26 (Human complementarity determining region 1 (CDR1) of heavy chain)
GYTFTEYT
SEQ ID NO: 27 (Human CDR2 of heavy chain)
NNPNTGGTT
SEQ ID NO: 28 (Human CDR3 of heavy chain)
SRSGNDVGWYFAV
SEQ ID NO: 29 (Constant region of heavy chain)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 30 (Chimeric heavy chain)
MGWSYIILFLVATATGVHSQVQLVQSGAEVKKPGASVKVSCKASGYTFTEYTMHW
VRQAPGQGLEWMGSNNPNTGGTTYNQKFKDRVTMTRDTSISTAYMELSRLRSDDTA
VYYCSRSGNDVGWYFAVWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHE
ALHNHYTQKSLSLSPGK-
“-” indicates a stop codon.
SEQ ID NO: 31 (Signal peptide of light chain)
MDMRVPAQLLGLLLLWLSGARC
SEQ ID NO: 32 (Murine FR1 of light chain)
EIVLTQSPATLSLSPGERATLSCTAT
SEQ ID NO: 33 (Murine FR2 of light chain)
MHWYQQKPGQAPRLLIY
SEQ ID NO: 34 (Murine FR3 of light chain)
KLASGIPARFSGSGSGTDFTLTISSLEPEDFAVYYC
SEQ ID NO: 35 (Murine FR4 of light chain)
FGGGTKLEIKR
SEQ ID NO: 36 (Human CDR1 of light chain)
SSVSY
SEQ ID NO: 37 (Human CDR2 of light chain)
DTS
SEQ ID NO: 38 (Human CDR3 of light chain)
QQWSSNPLT
SEQ ID NO: 39 (Constant region of light chain)
TVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
SEQ ID NO: 40 (Chimeric light chain)
MDMRVPAQLLGLLLLWLSGARCEIVLTQSPATLSLSPGERATLSCTATSSVSYMHWY
QQKPGQAPRLLIYDTSKLASGIPARFSGSGSGTDFTLTISSLEPEDFAVYYCQQWSSNP
LTFGGGTKLEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPVTKSF
NRGEC-
SEQ ID NO: 41 (Signal peptide of heavy chain)
MGWSYIILFLVATATGVHS
SEQ ID NO: 42 (Murine framework region 1 (FR1) of heavy chain)
QVQLVQSGAEVKKPGASVKVSCKAS
SEQ ID NO: 43 (Murine FR2 of heavy chain)
MHWVRQAPGQGLEWMGS
SEQ ID NO: 44 (Murine FR3 of heavy chain)
YNQKFKDRVTMTRDTSISTAYMELSRLRSDDTAVYYC
SEQ ID NO: 45 (Murine FR4 of heavy chain)
WGQGTLVTVSS
SEQ ID NO: 46 (Human complementarity determining region 1 (CDR1) of heavy chain)
GYTFTEYT
SEQ ID NO: 47 (Human CDR2 of heavy chain)
NNPNTGGTT
SEQ ID NO: 48 (Human CDR3 of heavy chain)
SRSGNDVGWYFAV
SEQ ID NO: 49 (Constant region of heavy chain)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 50 (Chimeric heavy chain)
MGWSYIILFLVATATGVHSQVQLVQSGAEVKKPGASVKVSCKASGYTFTEYTMHW
VRQAPGQGLEWMGSNNPNTGGTTYNQKFKDRVTMTRDTSISTAYMELSRLRSDDTA
VYYCSRSGNDVGWYFAVWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHE
ALHNHYTQKSLSLSPGK-
“-” indicates a stop codon.
SEQ ID NO: 51 (Signal peptide of light chain)
MDMRVPAQLLGLLLLWLSGARC
SEQ ID NO: 52 (Murine FR1= of light chain)
EIVLTQSPATLSLSPGERATLSCTAT
SEQ ID NO: 53 (Murine FR2 of light chain)
MHWYQQKPGLAPRLLIY
SEQ ID NO: 54 (Murine FR3 of light chain)
KLASGIPDRFSGSGSGTDFTLTISRLEPEDFAVYYC
SEQ ID NO: 55 (Murine FR4 of light chain)
FGGGTKLEIKR
SEQ ID NO: 56 (Human CDR1 of light chain)
SSVSY
SEQ ID NO: 57 (Human CDR2 of light chain)
DTS
SEQ ID NO: 58 (Human CDR3 of light chain)
QQWSSNPLT
SEQ ID NO: 59 (Constant region of light chain)
TVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC
SEQ ID NO: 60 (Chimeric light chain)
MDMRVPAQLLGLLLLWLSGARCEIVLTQSPATLSLSPGERATLSCTATSSVSYMHWY
QQKPGLAPRLLIYDTSKLASGIPDRFSGSGSGTDFTLTISRLEPEDFAVYYCQQWSSNP
LTFGGGTKLEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPVTKSF
NRGEC-

### REFERENCES

Hwang S Y, Park S, Kwon Y. Recent therapeutic trends and promising targets in triple negative breast cancer. Pharmacol Ther 2019; 199:30-57
Wein L, Loi S. Mechanisms of resistance of chemotherapy in early-stage triple negative breast cancer (TNBC). Breast 2017; 34 Suppl 1:S27-S30
Nedeljkovic M, Damjanovic A. Mechanisms of Chemotherapy Resistance in Triple-Negative Breast Cancer-How We Can Rise to the Challenge. Cells 2019; 8
Bardia A, Messersmith W A, Kio E A, Berlin J D, Vahdat L, Masters G A, et al. Sacituzumab govitecan, a Trop-2-directed antibody-drug conjugate, for patients with epithelial cancer: final safety and efficacy results from the phase I/II IMMU-132-01 basket trial. Ann Oncol 2021; 32:746-56
Espinosa Fernandez J R, Eckhardt B L, Lee J, Lim B, Pearson T, Seitz R S, et al. Identification of triple-negative breast cancer cell lines classified under the same molecular subtype using different molecular characterization techniques: Implications for translational research. PLOS One 2020; 15: e0231953 Mayer I A, Abramson V G, Lehmann B D, Pietenpol J A. New strategies for triple-negative breast cancer—deciphering the heterogeneity. Clin Cancer Res 2014; 20:782-90
Roncato F, Rruga F, Porcu E, Casarin E, Ronca R, Maccarinelli F, et al. Improvement and extension of anti-EGFR targeting in breast cancer therapy by integration with the Avidin-Nucleic-Acid-Nano-Assemblies. Nat Commun 2018; 9:4070
Loison R, Loirat D. [Pembrolizumab plus chemotherapy combination-first line in PD-L1 positive (CPS>/=10) metastatic and advanced triple-negative breast cancer]. Bull Cancer 2022; 109:387-9
Chen R, Jiang X, Sun D, Han G, Wang F, Ye M, et al. Glycoproteomics analysis of human liver tissue by combination of multiple enzyme digestion and hydrazide chemistry. J Proteome Res 2009; 8:651-61
1Bachawal S V, Jensen K C, Wilson K E, Tian L, Lutz A M, Willmann J K. Breast Cancer Detection by B7-H3-Targeted Ultrasound Molecular Imaging. Cancer Res 2015; 75:2501-9
Castellanos J R, Purvis I J, Labak C M, Guda M R, Tsung A J, Velpula K K, et al. B7-H3 role in the immune landscape of cancer. Am J Clin Exp Immunol 2017; 6:66-75
Seaman S, Zhu Z, Saha S, Zhang X M, Yang M Y, Hilton M B, et al. Eradication of Tumors through Simultaneous Ablation of CD276/B7-H3-Positive Tumor Cells and Tumor Vasculature. Cancer Cell 2017; 31:501-15 e8
Maeda N, Yoshimura K, Yamamoto S, Kuramasu A, Inoue M, Suzuki N, et al. Expression of B7-H3, a potential factor of tumor immune evasion in combination with the number of regulatory T cells, affects against recurrence-free survival in breast cancer patients. Ann Surg Oncol 2014; 21 Suppl 4: S546-54
DH O D, Mao Y, Mele D A. The Next Generation of Pattern Recognition Receptor Agonists: Improving Response Rates in Cancer Immunotherapy. Curr Med Chem 2020; 27:5654-74
Xu J, Li X, Du Y. Antibody-Pattern Recognition Receptor Agonist Conjugates: A Promising Therapeutic Strategy for Cancer. Adv Biol (Weinh) 2022; 6: e2101065
Sun H, Li Y, Zhang P, Xing H, Zhao S, Song Y, et al. Targeting toll-like receptor 7/8 for immunotherapy: recent advances and prospectives. Biomark Res 2022; 10:89
Patinote C, Karroum N B, Moarbess G, Cirnat N, Kassab I, Bonnet P A, et al. Agonist and antagonist ligands of toll-like receptors 7 and 8: Ingenious tools for therapeutic purposes. Eur J Med Chem 2020; 193:112238
Wu S Y, Xu Y, Chen L, Fan L, Ma X Y, Zhao S, et al. Combined angiogenesis and PD-1 inhibition for immunomodulatory TNBC: concept exploration and biomarker analysis in the FUTURE-C-Plus trial. Mol Cancer 2022; 21:84
Chi H, Li C, Zhao F S, Zhang L, Ng T B, Jin G, et al. Anti-tumor Activity of Toll-Like Receptor 7 Agonists. Front Pharmacol 2017; 8:304
Zuniga L A, Lessmann T, Uppal K, Bisek N, Hong E, Rasmussen C E, et al. Intratumoral delivery of TransCon( ) TLR7/8 Agonist promotes sustained anti-tumor activity and local immune cell activation while minimizing systemic cytokine induction. Cancer Cell Int 2022; 22:286
Lee S N, Jin S M, Shin H S, Lim Y T. Chemical Strategies to Enhance the Therapeutic Efficacy of Toll-like Receptor Agonist Based Cancer Immunotherapy. Acc Chem Res 2020; 53:2081-93
Teicher B A, Morris J. Antibody-drug Conjugate Targets, Drugs, and Linkers. Curr Cancer Drug Targets 2022; 22:463-529
Hafeez U, Parakh S, Gan H K, Scott A M. Antibody-Drug Conjugates for Cancer Therapy. Molecules 2020; 25
Polakis P. Antibody Drug Conjugates for Cancer Therapy. Pharmacol Rev 2016; 68:3-19
Mansoori B, Mohammadi A, Davudian S, Shirjang S, Baradaran B. The Different Mechanisms of Cancer Drug Resistance: A Brief Review. Adv Pharm Bull 2017; 7:339-48
26. Si Y, Kim S, Ou J, Lu Y, Ernst P, Chen K, et al. Anti-SSTR2 Antibody-drug Conjugate for Neuroendocrine Tumor Therapy. Cancer Gene Therapy. Cancer Gene Therapy 2020; 28:799-812
Si Y, Zhang Y, Guan J-S, Ngo H, Totoro A, Singh A, et al. Anti-CD47 Monoclonal Antibody-drug Conjugate: A Targeted Therapy to Treat Triple-negative Breast Cancers. Vaccines 2021; 9:882-895
Ou J, Si Y, Gah K Y, Song J, Patrick E, Flanigan D, et al. Process development of antibody-drug conjugation production for cancer treatment. PLOS ONE 2018; e0206246
Xu N, Ma C, Ou J, Sun W W, Zhou L, Hu H, et al. Comparative Proteomic Analysis of Three Chinese Hamster Ovary (CHO) Host Cells. Biochem Eng J 2017; 124:122-9
Xu N, Ou J, Si Y, Goh K, Flanigan D, Yang Y, et al. Proteomics insight into the production of monoclonal antibody. Biochemical Engineering Journal 2019; 145:177-185
Chen K, Si Y, Ou J, Guan J S, Kim S, Ernst P, et al. Antibody-Drug Conjugate to Treat Meningiomas. Pharmaceuticals (Basel) 2021; 14
Xu N, Ou J, Gilani A K, Zhou L, Liu X M. High-Level Expression of Recombinant IgG1 by CHO K1 Platform. Frontiers of Chemical Science & Engineering 2017; 9:376-80
Si Y, Guan J, Xu Y, Chen K, Kim S, Zhou L, et al. Dual-Targeted Extracellular Vesicles to Facilitate Combined Therapies for Neuroendocrine Cancer Treatment. Pharmaceutics 2020; 12:1079-90
Si Y, Kim S, Zhang E, Tang Y, Jaskula-Sztul R, Markert J M, et al. Targeted Exosomes for Drug Delivery: Biomanufacturing, Surface Tagging, and Validation. Biotechnol J 2019: e1900163
Si Y, Xu Y, Guan J, Chen K, Kim S, Yang E S, et al. Anti-EGFR antibody-drug conjugate for triple-negative breast cancer therapy. Eng Life Sci 2021; 21:37-44
Si Y, Zhang Y, Ngo H G, Guan J S, Chen K, Wang Q, et al. Targeted Liposomal Chemotherapies to Treat Triple-Negative Breast Cancer. Cancers (Basel) 2021; 13
Ou J, Si Y, Tang Y, Salzer G E, Lu Y, Kim S, et al. Novel biomanufacturing platform for large-scale and high-quality human T cells production. J Biol Eng 2019; 13:34
Yang S, Liu X. Cell culture process for Biologics manufacturing: recent development and trends. Journal of Biopharmaceuticals Bioprocessing 2013; 1:133-6
Wu L, Yi B, Wei S, Rao D, He Y, Naik G, et al. Loss of FOXP3 and TSC1 Accelerates Prostate Cancer Progression through Synergistic Transcriptional and Posttranslational Regulation of c-MYC. Cancer Res 2019; 79:1413-25
Schrors B, Boegel S, Albrecht C, Bukur T, Bukur V, Holtstrater C, et al. Multi-Omics Characterization of the 4T1 Murine Mammary Gland Tumor Model. Front Oncol 2020; 10:1195
Ou J, Si Y, Tang Y, Salzer G E, Lu Y, Kim S, et al. Novel biomanufacturing platform for large-scale and high-quality human T cells production. J Biol Eng 2019; 13:34-46
Chen K, Si Y, Ou J, Guan J S, Kim S, Ernst P, et al. Antibody-Drug Conjugate to Treat Meningiomas. Pharmaceuticals (Basel) 2021; 14:427-39
Topalian S L, Sznol M, McDermott D F, Kluger H M, Carvajal R D, Sharfman W H, et al. Survival, durable tumor remission, and long-term safety in patients with advanced melanoma receiving nivolumab. J Clin Oncol 2014; 32:1020-30
Huck B R, Kotzner L, Urbahns K. Small Molecules Drive Big Improvements in Immuno-Oncology Therapies. Angew Chem Int Ed Engl 2018; 57:4412-28
Galluzzi L, Buque A, Kepp O, Zitvogel L, Kroemer G. Immunogenic cell death in cancer and infectious disease. Nat Rev Immunol 2017; 17:97-111.

1.-92. (canceled)

93. A composition comprising: 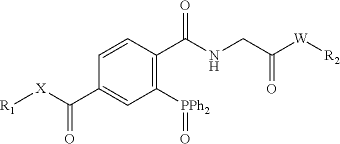
wherein R₁ comprises a means for binding CD276,
R₂ comprises an imidazoquinoline (IMQ),
X is selected from —O—, —S—, and —NH—, and
W is selected from —O—, —S—, and —NH—.

94. The composition of claim 93, wherein the means for binding CD276 comprises a heavy chain comprising a heavy chain complementarity determining region 1 (HCDR1) comprising SEQ ID NO: 6, a HCDR2 comprising SEQ ID NO: 7, and a HCDR3 comprising SEQ ID NO: 8; and a light chain comprising a light chain complementarity determining region 1 (LCDR1) comprising SEQ ID NO: 16, a LCDR2 comprising DTS, and a LCDR3 comprising SEQ ID NO: 18.

95. The composition of claim 94, wherein the heavy chain comprises a heavy chain framework 1 comprising SEQ ID NO: 2, a heavy chain framework 2 comprising SEQ ID NO: 3, a heavy chain framework 3 comprising SEQ ID NO: 4, and a heavy chain framework 4 comprising SEQ ID NO: 5; and the light chain comprises a heavy chain framework 1 comprising SEQ ID NO: 12, a heavy chain framework 2 comprising SEQ ID NO: 13, a heavy chain framework 3 comprising SEQ ID NO: 14, and a heavy chain framework 4 comprising SEQ ID NO: 15.

96. The composition of claim 93, wherein X is NH.

97. The composition of claim 93, wherein W is NH.

98. An antibody-drug conjugate comprising the composition of claim 93, wherein the means for binding is conjugated to a drug selected from the group consisting of: a topoisomerase inhibitor, an anti-microtubule agent, Altretamine, Bendamustine, Busulfan, Carmustine, Chlorambucil, Cyclophosphamide, Dacarbazine, Ifosfamide, Lomustine, Lurbinectedin, Mechlorethamine, Melphalan, Procarbazine, Streptozocin, Temozolomide, Thiotepa, Trabectedin, Carboplatin, Cisplatin, Oxaliplatin, Bleomycin, Dactinomycin, Mitomycin, Plicamycin, Methotrexate, Pemetrexed, Pralatrexate, Trimetrexate, Azathioprine, Cladribine, Fludarabine, Mercaptopurine, Thioguanine, Azacitidine, Capecitabine, Cytarabine, Decitabine, Floxuridine, Fluorouracil, Gemcitabine, Trifluridine/Tipracil, Aldesleukin (IL-2), Denileukin Diftitox, Interferon Gamma, Belinostat, Panobinostat, Romidepsin, Vorinostat, Abiraterone, Apalutamide, Bicalutamide, Cyproterone, Enzalutamide, Flutamide, Nilutamide, Anastrozole, Exemestane, Fulvestrant, Letrozole, Raloxifene, Tamoxifen, Toremifene, Degarelix, Goserelin, Histrelin, Leuprolide, Relugolix, Triptorelin, Lanreotide, Octreotide, Pasireotide, Abemaciclib, Acalabrutinib, Adagrasib, Afatinib, Alectinib, Alpelisib, Asciminib, Axitinib, Binimetinib, Bortezomib, Bosutinib, Brigatinib, Cabozantinib, Carfilzomib, Ceritinib, Cobimetinib, Copanlisib, Crizotinib, Dabrafenib, Dacomitinib, Dasatinib, Duvelisib, Enasidenib, Encorafenib, Entrectinib, Erdafitinib, Erlotinib, Fedratinib, Futibatinib, Gefitinib, Gilteritinib, Glasdegib, Ibrutinib, Idelalisib, Imatinib, Infigratinib, Ivosidenib, Ixazomib, Lapatinib, Larotrectinib, Lenvatinib, Lorlatinib, Midostaurin, Mobocertinib, Momelotinib, Neratinib, Nilotinib, Niraparib, Olaparib, Olutasidenib, Osimertinib, Pacritinib, Palbociclib, Pazopanib, Pemigatinib, Pexidartinib, Ponatinib, Regorafenib, Ribocicib, Ripretinib, Rucaparib, Ruxolitinib, Selumetinib, Sonidegib, Sorafenib, Sunitinib, Talazoparib, Tivozanib, Trametinib, Trilaciblib, Umbralisib, Vandetanib, Vemurafenib, Vismodegib, Zanubrutinib, Cabazitaxel, Docetaxel, Paclitaxel, Vinblastine, Vincristine, Vinorelbine, Asparaginase (Pegaspargase), Belzutifan, Bexarotene, Cedazuridine, Eribulin, Everolimus, Hydroxyurea, Ixabepilone, Lenalidomide, Mitotane, Omacetaxine, Pomalidomide, Selinexor, Tagraxofusp, Tazemetostat, Tebentafusp, Telotristat, Temsirolimus, Thalidomide, and Venetoclax.

99. The antibody-drug conjugate of claim 98, wherein the drug is the anti-microtubule agent, and the anti-microtubule agent is monomethyl auristatin F (MMAF).

100. The antibody-drug conjugate of claim 98, wherein the means for binding is further conjugated to the drug via a linker comprising:
(i) a phosphine-azide linker 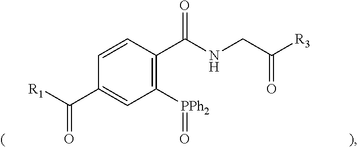
(ii) a N-succinimidyl S-acetylthioacetate sulfo-succinimidyl-4-(A-maleimidomethyl)cyclohexane-1-carboxylate (SATA sulfo-SMCC) linker 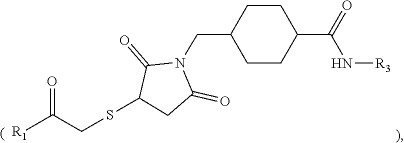
(iii) a sulfo-succinimidyl-4-(A-maleimidomethyl)cyclohexane-1-carboxylate (sulfo-SMCC) linker 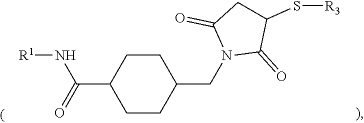
or
(iv) a dibromomaleimide (DBM) linker 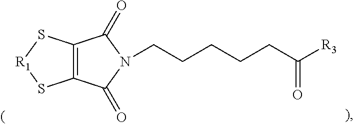
and R₃ comprises the drug.

101. A composition comprising the structure: 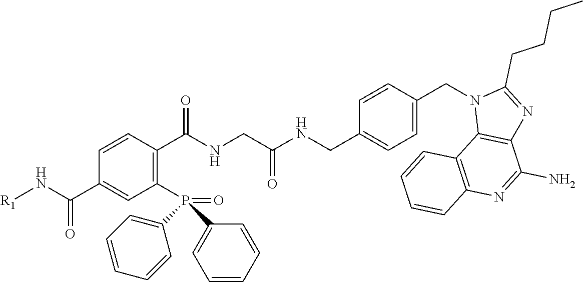
wherein R₁ comprises a means for binding CD276.

102. The composition of claim 101, wherein the means for binding CD276 comprises a heavy chain comprising a heavy chain complementarity determining region 1 (HCDR1) comprising SEQ ID NO: 6, a HCDR2 comprising SEQ ID NO: 7, and a HCDR3 comprising SEQ ID NO: 8; and a light chain comprising a light chain complementarity determining region 1 (LCDR1) comprising SEQ ID NO: 16, a LCDR2 comprising DTS, and a LCDR3 comprising SEQ ID NO: 18.

103. The composition of claim 102, wherein the heavy chain comprises a heavy chain framework 1 comprising SEQ ID NO: 2, a heavy chain framework 2 comprising SEQ ID NO: 3, a heavy chain framework 3 comprising SEQ ID NO: 4, and a heavy chain framework 4 comprising SEQ ID NO: 5; and the light chain comprises a heavy chain framework 1 comprising SEQ ID NO: 12, a heavy chain framework 2 comprising SEQ ID NO: 13, a heavy chain framework 3 comprising SEQ ID NO: 14, and a heavy chain framework 4 comprising SEQ ID NO: 15.

104. An antibody-drug conjugate comprising the composition of claim 101, wherein the means for binding is conjugated to a drug selected from the group consisting of: a topoisomerase inhibitor, an anti-microtubule agent, Altretamine, Bendamustine, Busulfan, Carmustine, Chlorambucil, Cyclophosphamide, Dacarbazine, Ifosfamide, Lomustine, Lurbinectedin, Mechlorethamine, Melphalan, Procarbazine, Streptozocin, Temozolomide, Thiotepa, Trabectedin, Carboplatin, Cisplatin, Oxaliplatin, Bleomycin, Dactinomycin, Mitomycin, Plicamycin, Methotrexate, Pemetrexed, Pralatrexate, Trimetrexate, Azathioprine, Cladribine, Fludarabine, Mercaptopurine, Thioguanine, Azacitidine, Capecitabine, Cytarabine, Decitabine, Floxuridine, Fluorouracil, Gemcitabine, Trifluridine/Tipracil, Aldesleukin (IL-2), Denileukin Diftitox, Interferon Gamma, Belinostat, Panobinostat, Romidepsin, Vorinostat, Abiraterone, Apalutamide, Bicalutamide, Cyproterone, Enzalutamide, Flutamide, Nilutamide, Anastrozole, Exemestane, Fulvestrant, Letrozole, Raloxifene, Tamoxifen, Toremifene, Degarelix, Goserelin, Histrelin, Leuprolide, Relugolix, Triptorelin, Lanreotide, Octreotide, Pasireotide, Abemaciclib, Acalabrutinib, Adagrasib, Afatinib, Alectinib, Alpelisib, Asciminib, Axitinib, Binimetinib, Bortezomib, Bosutinib, Brigatinib, Cabozantinib, Carfilzomib, Ceritinib, Cobimetinib, Copanlisib, Crizotinib, Dabrafenib, Dacomitinib, Dasatinib, Duvelisib, Enasidenib, Encorafenib, Entrectinib, Erdafitinib, Erlotinib, Fedratinib, Futibatinib, Gefitinib, Gilteritinib, Glasdegib, Ibrutinib, Idelalisib, Imatinib, Infigratinib, Ivosidenib, Ixazomib, Lapatinib, Larotrectinib, Lenvatinib, Lorlatinib, Midostaurin, Mobocertinib, Momelotinib, Neratinib, Nilotinib, Niraparib, Olaparib, Olutasidenib, Osimertinib, Pacritinib, Palbociclib, Pazopanib, Pemigatinib, Pexidartinib, Ponatinib, Regorafenib, Ribocicib, Ripretinib, Rucaparib, Ruxolitinib, Selumetinib, Sonidegib, Sorafenib, Sunitinib, Talazoparib, Tivozanib, Trametinib, Trilaciblib, Umbralisib, Vandetanib, Vemurafenib, Vismodegib, Zanubrutinib, Cabazitaxel, Docetaxel, Paclitaxel, Vinblastine, Vincristine, Vinorelbine, Asparaginase (Pegaspargase), Belzutifan, Bexarotene, Cedazuridine, Eribulin, Everolimus, Hydroxyurea, Ixabepilone, Lenalidomide, Mitotane, Omacetaxine, Pomalidomide, Selinexor, Tagraxofusp, Tazemetostat, Tebentafusp, Telotristat, Temsirolimus, Thalidomide, and Venetoclax.

105. The antibody-drug conjugate of claim 104, wherein the drug is the anti-microtubule agent, and the anti-microtubule agent is monomethyl auristatin F (MMAF).

106. The antibody-drug conjugate of claim 104, wherein the means for binding is conjugated to the drug via a phosphine-azide linker 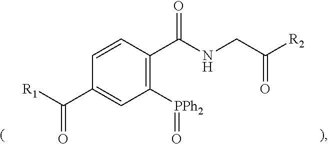
a N-succinimidyl S-acetylthioacetate sulfo-succinimidyl-4-(A-maleimidomethyl)cyclohexane-1-carboxylate (SATA sulfo-SMCC) linker 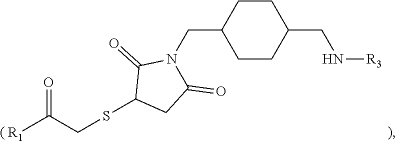
a sulfo-succinimidyl-4-(A-maleimidomethyl)cyclohexane-1-carboxylate (sulfo-SMCC) linker 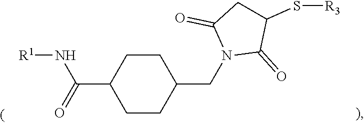
or a dibromomaleimide (DBM) linker

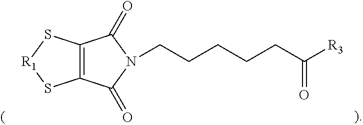

107. An antibody-drug conjugate having the following structure: 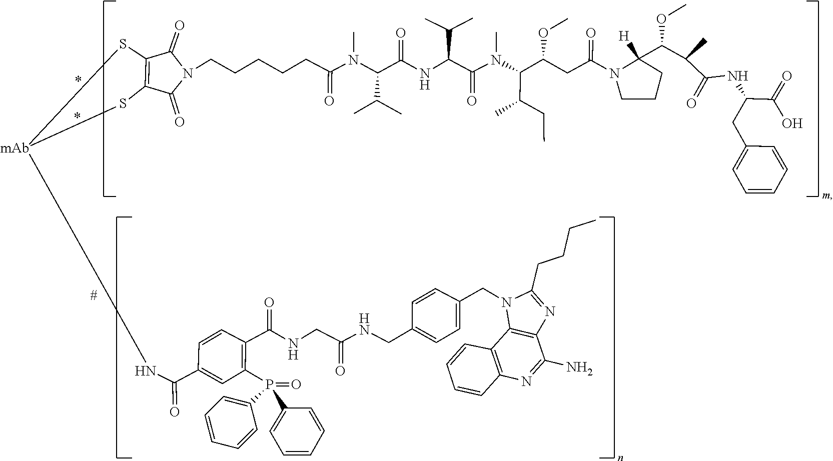
wherein m is 3-4, n is 7-14, and
mAb is a means for binding CD276.

108. The antibody-drug conjugate of claim 107, wherein the means for binding CD276 comprises a heavy chain comprising a heavy chain complementarity determining region 1 (HCDR1) comprising SEQ ID NO: 6, a HCDR2 comprising SEQ ID NO: 7, and a HCDR3 comprising SEQ ID NO: 8; and a light chain comprising a light chain complementarity determining region 1 (LCDR1) comprising SEQ ID NO: 16, a LCDR2 comprising DTS, and a LCDR3 comprising SEQ ID NO: 18.

109. The antibody-drug conjugate of claim 108, wherein the heavy chain comprises a heavy chain framework 1 comprising SEQ ID NO: 2, a heavy chain framework 2 comprising SEQ ID NO: 3, a heavy chain framework 3 comprising SEQ ID NO: 4, and a heavy chain framework 4 comprising SEQ ID NO: 5; and the light chain comprises a heavy chain framework 1 comprising SEQ ID NO: 12, a heavy chain framework 2 comprising SEQ ID NO: 13, a heavy chain framework 3 comprising SEQ ID NO: 14, and a heavy chain framework 4 comprising SEQ ID NO: 15.

110. The antibody-drug conjugate of claim 108, wherein the heavy chain comprises the heavy chain variable region of SEQ ID NO: 10, and the light chain comprises the light chain variable region of SEQ ID NO: 20.

111. A composition comprising 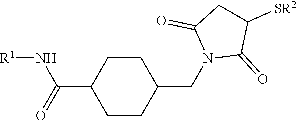
wherein R₁ comprises a means for binding CD276, and
R₂ comprises a drug selected from the group consisting of: an imidazoquinoline (IMQ), a topoisomerase inhibitor, an anti-microtubule agent, Altretamine, Bendamustine, Busulfan, Carmustine, Chlorambucil, Cyclophosphamide, Dacarbazine, Ifosfamide, Lomustine, Lurbinectedin, Mechlorethamine, Melphalan, Procarbazine, Streptozocin, Temozolomide, Thiotepa, Trabectedin, Carboplatin, Cisplatin, Oxaliplatin, Bleomycin, Dactinomycin, Mitomycin, Plicamycin, Methotrexate, Pemetrexed, Pralatrexate, Trimetrexate, Azathioprine, Cladribine, Fludarabine, Mercaptopurine, Thioguanine, Azacitidine, Capecitabine, Cytarabine, Decitabine, Floxuridine, Fluorouracil, Gemcitabine, Trifluridine/Tipracil, Aldesleukin (IL-2), Denileukin Diftitox, Interferon Gamma, Belinostat, Panobinostat, Romidepsin, Vorinostat, Abiraterone, Apalutamide, Bicalutamide, Cyproterone, Enzalutamide, Flutamide, Nilutamide, Anastrozole, Exemestane, Fulvestrant, Letrozole, Raloxifene, Tamoxifen, Toremifene, Degarelix, Goserelin, Histrelin, Leuprolide, Relugolix, Triptorelin, Lanreotide, Octreotide, Pasireotide, Abemaciclib, Acalabrutinib, Adagrasib, Afatinib, Alectinib, Alpelisib, Asciminib, Axitinib, Binimetinib, Bortezomib, Bosutinib, Brigatinib, Cabozantinib, Carfilzomib, Ceritinib, Cobimetinib, Copanlisib, Crizotinib, Dabrafenib, Dacomitinib, Dasatinib, Duvelisib, Enasidenib, Encorafenib, Entrectinib, Erdafitinib, Erlotinib, Fedratinib, Futibatinib, Gefitinib, Gilteritinib, Glasdegib, Ibrutinib, Idelalisib, Imatinib, Infigratinib, Ivosidenib, Ixazomib, Lapatinib, Larotrectinib, Lenvatinib, Lorlatinib, Midostaurin, Mobocertinib, Momelotinib, Neratinib, Nilotinib, Niraparib, Olaparib, Olutasidenib, Osimertinib, Pacritinib, Palbociclib, Pazopanib, Pemigatinib, Pexidartinib, Ponatinib, Regorafenib, Ribocicib, Ripretinib, Rucaparib, Ruxolitinib, Selumetinib, Sonidegib, Sorafenib, Sunitinib, Talazoparib, Tivozanib, Trametinib, Trilaciblib, Umbralisib, Vandetanib, Vemurafenib, Vismodegib, Zanubrutinib, Cabazitaxel, Docetaxel, Paclitaxel, Vinblastine, Vincristine, Vinorelbine, Asparaginase (Pegaspargase), Belzutifan, Bexarotene, Cedazuridine, Eribulin, Everolimus, Hydroxyurea, Ixabepilone, Lenalidomide, Mitotane, Omacetaxine, Pomalidomide, Selinexor, Tagraxofusp, Tazemetostat, Tebentafusp, Telotristat, Temsirolimus, Thalidomide, and Venetoclax.

112. The composition of claim 111, comprising the structure: [chemical structure]


SEQUENCE LISTING (60 sequences)

SEQ ID NO: 1 - protein, 18 aa, synthetic construct
  REGION 1..18: note = Description of sequence: Signal peptide of heavy chain
MGWSYIILFLVATATGVH

SEQ ID NO: 2 - protein, 25 aa, Mus musculus [sequence omitted]

SEQ ID NO: 3 - protein, 17 aa, Mus musculus
  REGION 1..17: note = Description of sequence: Murine FR2 of heavy chain
MHWVRQAPGQGLEWMGS

SEQ ID NO: 4 - protein, 37 aa, Mus musculus
  REGION 1..37: note = Description of sequence: Murine FR3 of heavy chain
YNQKFKDRVTMTRDTSISTAYMELSRLRSDDTAVYYC

SEQ ID NO: 5 - protein, 11 aa, Mus musculus
  REGION 1..11: note = Description of sequence: Murine FR4 of heavy chain
WGQGTLVTVSS

SEQ ID NO: 6 - protein, 8 aa, Homo sapiens
  REGION 1..8: note = Description of sequence: Human complementarity determining region 1 (CDR1) of heavy chain
GYTFTEYT

SEQ ID NO: 7 - protein, 9 aa, Homo sapiens
  REGION 1..9: note = Description of sequence: Human CDR2 of heavy chain
NNPNTGGTT

SEQ ID NO: 8 - protein, 13 aa, Homo sapiens
  REGION 1..13: note = Description of sequence: Human CDR3 of heavy chain
SRSGNDVGWYFAV

SEQ ID NO: 9 - protein, 330 aa, synthetic construct [sequence omitted]

SEQ ID NO: 10 - protein, 469 aa, synthetic construct
  REGION 1..469: note = Description of sequence: Chimeric heavy chain
MGWSYIILFLVATATGVHSQVQLVQSGAEVKKPGASVKVSCKASGYTFTEYTMHWVRQAPGQGLEWMGSNNPNTGGTTYNQKFKDRVTMTRDTSISTAYMELSRLRSDDTAVYYCSRSGNDVGWYFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 22 aa, synthetic construct
  REGION 1..22: note = Description of sequence: Signal peptide of light chain
MDMRVPAQLLGLLLLWLSGARC

SEQ ID NO: 12 - protein, 26 aa, Mus musculus
  REGION 1..26: note = Description of sequence: Murine FR1 of light chain
EIVMTQSPATLSVSPGERATLSCTAT

SEQ ID NO: 13 - protein, 17 aa, Mus musculus
  REGION 1..17: note = Description of sequence: Murine FR2 of light chain
MHWYQQKPGQAPRLLIY

SEQ ID NO: 14 - protein, 36 aa, Mus musculus
  REGION 1..36: note = Description of sequence: Murine FR3 of light chain
KLASGIPARFSGSGSGTEFTLTISSLQSEDFAVYYC

SEQ ID NO: 15 - protein, 11 aa, Mus musculus
  REGION 1..11: note = Description of sequence: Murine FR4 of light chain
FGGGTKLEIKR

SEQ ID NO: 16 - protein, 5 aa, Homo sapiens
  REGION 1..5: note = Description of sequence: Human CDR1 of light chain
SSVSY

SEQ ID NO: 17 - skipped

SEQ ID NO: 18 - protein, 9 aa, Homo sapiens
  REGION 1..9: note = Description of sequence: Human CDR3 of light chain
QQWSSNPLT

SEQ ID NO: 19 - protein, 106 aa, synthetic construct
  REGION 1..106: note = Description of sequence: Constant region of light chain
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 235 aa, synthetic construct
  REGION 1..235: note = Description of sequence: Chimeric light chain
MDMRVPAQLLGLLLLWLSGARCEIVMTQSPATLSVSPGERATLSCTATSSVSYMHWYQQKPGQAPRLLIYDTSKLASGIPARFSGSGSGTEFTLTISSLQSEDFAVYYCQQWSSNPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 19 aa, synthetic construct
  REGION 1..19: note = Description of sequence: Signal peptide of heavy chain
MGWSYIILFLVATATGVHS

SEQ ID NO: 22 - protein, 25 aa, Mus musculus [sequence omitted]

SEQ ID NO: 23 - protein, 17 aa, Mus musculus
  REGION 1..17: note = Description of sequence: Murine FR2 of heavy chain
MHWVRQAPGQGLEWMGS

SEQ ID NO: 24 - protein, 37 aa, Mus musculus; identical to SEQ ID NO: 4
  REGION 1..37: note = Description of sequence: Murine FR3 of heavy chain

SEQ ID NO: 25 - protein, 11 aa, Mus musculus
  REGION 1..11: note = Description of sequence: Murine FR4 of heavy chain
WGQGTLVTVSS

SEQ ID NO: 26 - protein, 8 aa, Homo sapiens
  REGION 1..8: note = Description of sequence: Human complementarity determining region 1 (CDR1) of heavy chain
GYTFTEYT

SEQ ID NO: 27 - protein, 9 aa, Homo sapiens
  REGION 1..9: note = Description of sequence: Human CDR2 of heavy chain
NNPNTGGTT

SEQ ID NO: 28 - protein, 13 aa, Homo sapiens
  REGION 1..13: note = Description of sequence: Human CDR3 of heavy chain
SRSGNDVGWYFAV

SEQ ID NO: 29 - protein, 330 aa, synthetic construct [sequence omitted]

SEQ ID NO: 30 - protein, 469 aa, synthetic construct; identical to SEQ ID NO: 10
  REGION 1..469: note = Description of sequence: Chimeric heavy chain

SEQ ID NO: 31 - protein, 22 aa, synthetic construct
  REGION 1..22: note = Description of sequence: Signal peptide of light chain
MDMRVPAQLLGLLLLWLSGARC

SEQ ID NO: 32 - protein, 26 aa, Mus musculus
  REGION 1..26: note = Description of sequence: Murine FR1 of light chain
EIVLTQSPATLSLSPGERATLSCTAT

SEQ ID NO: 33 - protein, 17 aa, Mus musculus
  REGION 1..17: note = Description of sequence: Murine FR2 of light chain
MHWYQQKPGQAPRLLIY

SEQ ID NO: 34 - protein, 36 aa, Mus musculus
  REGION 1..36: note = Description of sequence: Murine FR3 of light chain
KLASGIPARFSGSGSGTDFTLTISSLEPEDFAVYYC

SEQ ID NO: 35 - protein, 11 aa, Mus musculus
  REGION 1..11: note = Description of sequence: Murine FR4 of light chain
FGGGTKLEIKR

SEQ ID NO: 36 - protein, 5 aa, Homo sapiens
  REGION 1..5: note = Description of sequence: Human CDR1 of light chain
SSVSY

SEQ ID NO: 37 - skipped

SEQ ID NO: 38 - protein, 9 aa, Homo sapiens
  REGION 1..9: note = Description of sequence: Human CDR3 of light chain
QQWSSNPLT

SEQ ID NO: 39 - protein, 106 aa, synthetic construct; identical to SEQ ID NO: 19
  REGION 1..106: note = Description of sequence: Constant region of light chain

SEQ ID NO: 40 - protein, 235 aa, synthetic construct
  REGION 1..235: note = Description of sequence: Chimeric light chain
MDMRVPAQLLGLLLLWLSGARCEIVLTQSPATLSLSPGERATLSCTATSSVSYMHWYQQKPGQAPRLLIYDTSKLASGIPARFSGSGSGTDFTLTISSLEPEDFAVYYCQQWSSNPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 19 aa, synthetic construct
  REGION 1..19: note = Description of sequence: Signal peptide of heavy chain
MGWSYIILFLVATATGVHS

SEQ ID NO: 42 - protein, 25 aa, Mus musculus [sequence omitted]

SEQ ID NO: 43 - protein, 17 aa, Mus musculus
  REGION 1..17: note = Description of sequence: Murine FR2 of heavy chain
MHWVRQAPGQGLEWMGS

SEQ ID NO: 44 - protein, 37 aa, Mus musculus; identical to SEQ ID NO: 4
  REGION 1..37: note = Description of sequence: Murine FR3 of heavy chain

SEQ ID NO: 45 - protein, 11 aa, Mus musculus
  REGION 1..11: note = Description of sequence: Murine FR4 of heavy chain
WGQGTLVTVSS

SEQ ID NO: 46 - protein, 8 aa, Homo sapiens
  REGION 1..8: note = Description of sequence: Human complementarity determining region 1 (CDR1) of heavy chain
GYTFTEYT

SEQ ID NO: 47 - protein, 9 aa, Homo sapiens
  REGION 1..9: note = Description of sequence: Human CDR2 of heavy chain
NNPNTGGTT

SEQ ID NO: 48 - protein, 13 aa, Homo sapiens
  REGION 1..13: note = Description of sequence: Human CDR3 of heavy chain
SRSGNDVGWYFAV

SEQ ID NO: 49 - protein, 330 aa, synthetic construct [sequence omitted]

SEQ ID NO: 50 - protein, 469 aa, synthetic construct; identical to SEQ ID NO: 10
  REGION 1..469: note = Description of sequence: Chimeric heavy chain

SEQ ID NO: 51 - protein, 22 aa, synthetic construct
  REGION 1..22: note = Description of sequence: Signal peptide of light chain
MDMRVPAQLLGLLLLWLSGARC

SEQ ID NO: 52 - protein, 26 aa, Mus musculus
  REGION 1..26: note = Description of sequence: Murine FR1 of light chain
EIVLTQSPATLSLSPGERATLSCTAT

SEQ ID NO: 53 - protein, 17 aa, Mus musculus
  REGION 1..17: note = Description of sequence: Murine FR2 of light chain
MHWYQQKPGLAPRLLIY

SEQ ID NO: 54 - protein, 36 aa, Mus musculus
  REGION 1..36: note = Description of sequence: Murine FR3 of light chain
KLASGIPDRFSGSGSGTDFTLTISRLEPEDFAVYYC

SEQ ID NO: 55 - protein, 11 aa, Mus musculus
  REGION 1..11: note = Description of sequence: Murine FR4 of light chain
FGGGTKLEIKR

SEQ ID NO: 56 - protein, 5 aa, Homo sapiens
  REGION 1..5: note = Description of sequence: Human CDR1 of light chain
SSVSY

SEQ ID NO: 57 - skipped

SEQ ID NO: 58 - protein, 9 aa, Homo sapiens
  REGION 1..9: note = Description of sequence: Human CDR3 of light chain
QQWSSNPLT

SEQ ID NO: 59 - protein, 106 aa, synthetic construct; identical to SEQ ID NO: 19
  REGION 1..106: note = Description of sequence: Constant region of light chain

SEQ ID NO: 60 - protein, 235 aa, synthetic construct
  REGION 1..235: note = Description of sequence: Chimeric light chain
MDMRVPAQLLGLLLLWLSGARCEIVLTQSPATLSLSPGERATLSCTATSSVSYMHWYQQKPGLAPRLLIYDTSKLASGIPDRFSGSGSGTDFTLTISRLEPEDFAVYYCQQWSSNPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC